SEQUENCE LISTING (140 sequences)

SEQ ID NO: 1 - protein, 201 aa, Artificial Sequence
  Synthetic HIV-1 p28TEV
MEPVDPRLEPWKHPGSQPKTACTNCYCKKCCFHCQVCFITKALGISYGRKKRRQRRRAHQNSQTHQASLSKHLKCTDLKNDTNTNSSSGRMIMEKGEIKNCSFNISTSIRDPPPNPEGTRQARRNRRRRWRERQRQIHSISERILGTYLGRSAEPVPLQLPPLERLTLDCNEDCGTSGTQGVGSPQILVESPTVLESGTKE

SEQ ID NO: 2 - DNA, 606 bp, Artificial Sequence
  DNA encoding synthetic HIV-1 p28TEV
atggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagagctcatcagaacagtcagactcatcaagcttctctatcaaagcatttaaagtgcactgatttgaagaatgatactaataccaatagtagtagcgggagaatgataatggagaaaggagagataaaaaactgctctttcaatatcagcacaagcataagagacccacctcccaatcccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatag

SEQ ID NO: 3 - RNA, 3 nt, Artificial Sequence
  Splice acceptor site
nag

SEQ ID NO: 4 - RNA, 3 nt, Artificial Sequence
  Donor splice site
nag

SEQ ID NO: 5 - RNA, 3 nt, Artificial Sequence
  Splice site
agg

SEQ ID NO: 6 - RNA, 3 nt, Artificial Sequence
  Splice site
cag

SEQ ID NO: 7 - RNA, 3 nt, Artificial Sequence
  Acceptor splice site
uag

SEQ ID NO: 8 - RNA, 3 nt, Artificial Sequence
  Donor splice site
gag

SEQ ID NO: 9 - DNA, 33 bp, Artificial Sequence
  PCR primer
ccccaagcttccatggcactatgctgtttcgcc

SEQ ID NO: 10 - DNA, 29 bp, Artificial Sequence
  PCR primer
ccgaattcaggttctctgggtggggaagg

SEQ ID NO: 11 - DNA, 24 bp, Artificial Sequence
  Splice-specific PCR primer
ctctgtgtttcactgaagtgcact

SEQ ID NO: 12 - DNA, 6 bp, Artificial Sequence
  PCR
tcactg

SEQ ID NO: 13 - DNA, 6 bp, Artificial Sequence
  Acceptor splice site
agttta

SEQ ID NO: 14 - DNA, 104 bp, Human immunodeficiency virus type 1
tggtagaacagatgcatgaggatataatcagtttatgggatcaaagcctaaagccatgtgtaaaattaaccccactctgtgttagtttaaagtgcactgatttg

SEQ ID NO: 15 - DNA, 104 bp, Human immunodeficiency virus type 1
tggtagaacagatgcatgaggatataatcagtttatgggatgaaagcctaaagccatgtgtaaaactaacccctctctgtgttactttaaactgtactgagata

SEQ ID NO: 16 - DNA, 110 bp, Human immunodeficiency virus type 1
tggtagaacagatgcaggaggatataatcagtttatgggaggaaagcctaaagccatgtgtaaagctaactcctctctgtatcactttaaactgtactaatgtaaacagt

SEQ ID NO: 17 - DNA, 125 bp, Human immunodeficiency virus type 1
tggtggaacaaatgcatgaggatataatcagtttatgggatgaaagcctaaagccatgtgtaaagctaacccctctctgtgttaccttaaactgtactgatgtaaccaacaaaggcaacaaaaga

SEQ ID NO: 18 - DNA, 104 bp, Human immunodeficiency virus type 1
tggtagaacagatgcatgaggatataatcagtttatgggatgaaagcctaaagccatgtgtaaagctaacccctctctgtgttactttaaactgtactgatgca

SEQ ID NO: 19 - DNA, 110 bp, Human immunodeficiency virus type 1
tggtagaacagatgcatacagatataatcagtttatgggatcagagcttaaagccatgtgtaaagttaaccccactctgtgttactctagactgtagtagtgtaaatgca

SEQ ID NO: 20 - DNA, 104 bp, Human immunodeficiency virus type 1
tggtagagcagatgcagacagacataatcagtttatgggatcaaagcttaaagccatgtgtaaaattaaccccactctgtgttactctagactgtagtaatata

SEQ ID NO: 21 - DNA, 104 bp, Human immunodeficiency virus type 1
tggtggagcagatgcatacagacataatcagtttatgggatcaaagcttgaaaccatgtgtaaaattaaccccactctgtgttactctaaactgtactaatgtc

SEQ ID NO: 22 - DNA, 104 bp, Human immunodeficiency virus type 1
tggtagaccagatgcaggaagatataatcagtgtatgggatgaaagcttaaagccatgtgtaaagataacccctctctgtgtcactttaaattgtagcgacgtc

SEQ ID NO: 23 - DNA, 104 bp, Human immunodeficiency virus type 1
tggtagaccagatgcaggaagatataatcagtgtatgggatgaaagcttaaagccatgtgtaaagataacccctctatgtgtcactttaaattgtagtaacatc

SEQ ID NO: 24 - DNA, 125 bp, Human immunodeficiency virus type 1
tggtagagcagatgcatacagatataatcagtttatgggatgaaagcctaaaaccttgtgtagagttaacccctctctgtgtcactttaaattgtactgattacaaaggaaccaatagcaccaat

SEQ ID NO: 25 - DNA, 125 bp, Human immunodeficiency virus type 1
tggtagagcagatgcatacagatataatcagtttatgggatgaaagcctaaaaccatgtgtaaagttaacccctctctgtgtcactctaacttgtaccaatgtaactaataaccgtaccaatgca

SEQ ID NO: 26 - DNA, 104 bp, Human immunodeficiency virus type 1
tggtagaacaaatgcaggaagacattattagtttatgggaccagagtcttaaaccttgtgttcaaatgactttcctgtgtgtacaaatggagtgtacaaacata

SEQ ID NO: 27 - DNA, 104 bp, Human immunodeficiency virus type 1
tggtggaccaaatgcatgaagacatcattagtttgtgggaacagagtttaaagccttgtgagaaaatgactttcttatgtgtacaaatgaactgtgtagatctg

SEQ ID NO: 28 - DNA, 130 bp, Human immunodeficiency virus type 1
tggtagaacaaatgcaggaagacattattagtttatgggaacagagcttaaaaccttgtgttcaaatgactttcctgtgtgtacaaatgaattgtacaaactatgtacaagggaattatacaaacaatag

SEQ ID NO: 29 - DNA, 130 bp, Human immunodeficiency virus type 1
tggtagaacaaatgcaggaagatattattagtttatgggaacagagtttaaaaccttgtgttcaaatgacttttctgtgtgtacaaatgaattgtacaaatgtaaatgatgagaccaacagctccgtaaa

SEQ ID NO: 30 - DNA, 130 bp, Human immunodeficiency virus type 1
tggcagaccaaatgcaagaggatattatcagtctgtgggaacagagcttaaagccctgtgttaaattaaccccattatgtgtaactatgctttgtaacgatagctatggggaggaaaggaacaatacaaa

SEQ ID NO: 31 - DNA, 130 bp, Human immunodeficiency virus type 1
tggcagaacaaatgcaagaggacattatcagtttatgggaacagagcttaaagccctgtgttaaattaaccccattatgtgtaactatgctttgtaacaatagcaatgggaatagtgcagggaatagtac

SEQ ID NO: 32 - DNA, 130 bp, Human immunodeficiency virus type 1
  misc_feature (119)..(119): n is unknown
tggtagagcagatgcaggaggatgtaatcagtttatgggatcaaagtctaaagccatgtgtaaagttaactcctctctgcgttactttaaattgtaccaatgctaatttgaccaatgtnaataacnnnnn

SEQ ID NO: 33 - DNA, 119 bp, Human immunodeficiency virus type 1
tggtagagcagatgcaggaggatgtaatcagtttatgggatcaaagtctaaagccatgtgtaaagttaactcctctctgcgttactttaaattgtaccaatgctaattggaccaatgct

SEQ ID NO: 34 - DNA, 110 bp, Human immunodeficiency virus type 1
tggtagagcagatgcaggaggatgtaatcagtttatgggatcaaagtctaaagccatgtgtaaagttaactcctctctgcgttactttaaattgtaccaatgctaatttg

SEQ ID NO: 35 - DNA, 104 bp, Human immunodeficiency virus type 1
tggtagagcagatgcaggaggatgtaatcagtttatgggatcaaagtctacagccatgtgtaaagttaactcctctctgtgttactttacattgtaccaaggct

SEQ ID NO: 36 - DNA, 125 bp, Human immunodeficiency virus type 1
tggcagagcagatgcaggaggatgtaattagtttatgggatcaaagtctaaagccatgtgtaaagttaactcctctctgcgttactttaaattgtaccaatgtcaatgctaccaatgtcagtaac

SEQ ID NO: 37 - DNA, 130 bp, Human immunodeficiency virus type 1
tggtagagcagatgcaggaggatgtaatcagtttatgggatcaaagtctaaagccatgtgtaaagttaactcctctctgcgttactttaaattgtaccactgctaactttaccaattttaacttgaccaa

SEQ ID NO: 38 - DNA, 130 bp, Human immunodeficiency virus type 1
tggtagagcagatgcaagaggatgtaatcagtttatgggatcaaagtctaaagccatgtgtaaagttaactcctctttgcgtcactttacattgtaccaaggctaaactgaatgacacttataacggaac

SEQ ID NO: 39 - DNA, 130 bp, Human immunodeficiency virus type 1
tggtagagcagatgcaggaggatgtaatcagtttatgggatcaaagtctacagccatgtgtaaagttaactcctctctgcgttactttacattgtaccactgctaaattgaccaatgtcactaacataac

SEQ ID NO: 40 - DNA, 130 bp, Human immunodeficiency virus type 1
tggtagagcagatgcaggaggatgtaatcagtttatgggatcaaagtctaaaaccatgtgtaaagttaactcctctctgcgttactttaaattgtaccaatgctaatttgaccaatgtcaataacaatgt

SEQ ID NO: 41 - DNA, 130 bp, Human immunodeficiency virus type 1
tggtagagcagatgcaggaggatgtaatcagtttatgagatcaaagtctaaagccatgtgtaaagttaactcctctctgtgttactttagattgtaccaaagctgatttttacaccacaaaatttaacac

SEQ ID NO: 42 - DNA, 128 bp, Human immunodeficiency virus type 1
  misc_feature (8)..(8): n is unknown
tggtagancagatgcatgaagatataattagtctatgggaccaaagcctaaagccatgtgtaaagttaacccctctctgcgttactttagattgtcataacttcancnnnnnnnnnnnnnnnnnnnnn

SEQ ID NO: 43 - DNA, 116 bp, Human immunodeficiency virus type 1
tggtagagcagatgcatgaagatataattagtctatgggatcaaagcctaaagccatgtgtacagttaacccctctctgtgttactttacattgtcaggataacctcactagcagc

SEQ ID NO: 44 - DNA, 107 bp, Human immunodeficiency virus type 1
tggtagagcagatgcatgtagatataattagtctatgggaccaaagcttaaagccatgtgtaaagttaacccctctctgcgttactctagattgtcagaactttaaa

SEQ ID NO: 45 - DNA, 116 bp, Human immunodeficiency virus type 1
tggtagaacagatgcatgaagatataattagtctatgggaccaaagcctaaagccatgtgtaaagttaacccctctctgtgttactttagattgtcataatgtcaacagcagcaat

SEQ ID NO: 46 - DNA, 104 bp, Human immunodeficiency virus type 1
tggtagaacagatgcatgaagatataattagtctatgggaccaaagcctaaagccatgtgtagagttaacccctctctgcgttactttagattgttataacgtc

SEQ ID NO: 47 - DNA, 104 bp, Human immunodeficiency virus type 1
tggtagaacagatgcatgaagatataattagtctatgggaccaaagcctaaagccatgtgtaaagttaacccctctctgcgttactttagattgtcataatttc

SEQ ID NO: 48 - DNA, 104 bp, Human immunodeficiency virus type 1
tggtagaacaaatgcatgtagatataattagcctatgggatcaaagtctaaagccatgtgtaaagttaacccctctctgtgttactttagagtgtcataactat

SEQ ID NO: 49 - DNA, 128 bp, Human immunodeficiency virus type 1
tggtagagcagatgaatgaagatataattagtctatgggaccaaagcctaaagccatgtgtaaagttaacccctctctgcgttactttaaattgtcataacatcaccaacacctctagcggcaacaat

SEQ ID NO: 50 - DNA, 113 bp, Human immunodeficiency virus type 1
tggtagagcagatgcatatagatataattagtctatgggaacaaagcctaaagccatgtgtaaagttgacccctctctgcgtcactctagattgccatgactacaacagcacc

SEQ ID NO: 51 - DNA, 104 bp, Human immunodeficiency virus type 1
tggtagaacagatgcatgaggatataatcagtttatgggatcaaagcctaaagccatgtgtaaagttaaccccactctgtgttactttaaattgcactgaagtg

SEQ ID NO: 52 - DNA, 104 bp, Human immunodeficiency virus type 1
tggtagaacagatgcatgaggatataatcagtttatgggatcaaagcctaaagccatgtgtaaagttaaccccactctgtgttactttaaattgcactgatttg

SEQ ID NO: 53 - DNA, 104 bp, Human immunodeficiency virus type 1
tggtagaacagatgcatgaggatataatcagtttatgggaggaaggcctaaaaccatgtgtaaagctaacccctctctgtgttgctttaaactgtggtgatgca

SEQ ID NO: 54 - DNA, 119 bp, Human immunodeficiency virus type 1
cggtagaacagatgcatgaggatataatcagtttatgggatgaaggcctaaaaccatgtgtaaagctaacccctctctgtgttgctttaaactgtagtaatgcaactataatcattaat

SEQ ID NO: 55 - DNA, 104 bp, Human immunodeficiency virus type 1
tggtagaacagatgcatgaggatataatcagtttatggaatgaaggcctaaaaccatgtgcaaagctaacctctctctgtgttacttttacatgtattaatgca

SEQ ID NO: 56 - DNA, 124 bp, Human immunodeficiency virus type 1
tggtagaacaaatgcatacagatataattagtttatgggatcaaagcttaaagccatgtgtaaagttaaccccactctgtgttactctaaactgtactgatttcaaggccaattccactgccaa

SEQ ID NO: 57 - DNA, 109 bp, Human immunodeficiency virus type 1
tggtagaacaaatgcatgcagatataatcagtttatgggatcaaagcttaaagtcatgtgtaaagttaaccccactctgtgttactctaaactgtactgatgccaccag

SEQ ID NO: 58 - DNA, 104 bp, Human immunodeficiency virus type 1
tggtacaacagatgcatgaggatataatcagtttatgggatgaaagcctaaagccatgtgtaaagttgacccctctctgtgttactttaaactgtactaacgta

SEQ ID NO: 59 - DNA, 124 bp, Human immunodeficiency virus type 1
tggtagaccagatgcatgaggatataatcagtttatgggatgaaagtctaaagccatgtgtaaagctaacccctctctgcgttactttaacatgtactaatgctacattaggtaacaaaacatt

SEQ ID NO: 60 - DNA, 130 bp, Human immunodeficiency virus type 1
tggtagaacagatgcatgaggatataattagtttatgggatgaaagcctaaagccatgtgtaaagctaacccctctctgtgttactttaatctgtactaatataaatataaccagtactaatagcaccaa

SEQ ID NO: 61 - DNA, 130 bp, Human immunodeficiency virus type 1
tggtagaacagatgcatgaggatataattagtttatgggaggaaagcctaaagccatgtgtaaagctaacccctctctgtgttactttaaactgtactaatgtcaatgcaactaaatatagtaatgaaac

SEQ ID NO: 62 - DNA, 130 bp, Human immunodeficiency virus type 1
tggtggatcagatgcatgaggatataatcagtttatgggatcaaggcctaaaaccatgtataaaattaaccccactctgtgtcactttaaactgcaatggcattaatgccactaatgacactgttggtga

SEQ ID NO: 63 - DNA, 130 bp, Human immunodeficiency virus type 1
tggtggatcagatgcatgaggatataatcagtttatgggatcaaggcttaaaaccatgtgtaaaattaaccccactctgtgtcactttgaactgcagtgaatggaatgcgactaaccargagggtaggac

SEQ ID NO: 64 - DNA, 130 bp, Human immunodeficiency virus type 1
tggtagatcagatgcatgaggatataatcagtttatgggatcaaagcctaaaaccatgtgtaaaattaaccccactctgtgtcactttaaactgcagtgatgttgcctctaacgccaacgtcactagtgg

SEQ ID NO: 65 - DNA, 122 bp, Human immunodeficiency virus type 1
tggtagaacagatgcatgaggatataatcagtttatgggatgaaagcctaaaaccatgtgtaaagctaacccctctctgtgtcactttaaactgtgctgaagtaacctcctacaacactaca

SEQ ID NO: 66 - DNA, 130 bp, Human immunodeficiency virus type 1
tggtagaacagatgcatgaggacataatcagtttatgggatgaaagcctaaaaccatgtgtaaagctgacccctctttgtgttactttaaactgtactaatctaaatgtaaccaacagcacggctgtagg

SEQ ID NO: 67 - DNA, 122 bp, Human immunodeficiency virus type 1
tggtagaacagatgcatgaggatataattagtttatgggatgaaagtctaaaaccatgtgtaaagctaacccctctctgtgttactttaaaatgtgctaatgttacttctaatagtactgcg

SEQ ID NO: 68 - DNA, 116 bp, Human immunodeficiency virus type 1
tggtagaacagatgcatgaggatgtaatcagcttatgggatgaaagcttaaaaccatgtgtaaagctaacccctctctgtgttgctttaaattgtactgatgcaagggataacgca

SEQ ID NO: 69 - DNA, 113 bp, Simian immunodeficiency virus chimpanzee
tggtggaccaaatgcatgaagacattatcagtctctgggatcagagtctcaagccttgtgtaaagttgacccctttatgtgtaaccttgcagtgcagtaaggctaactttagc

SEQ ID NO: 70 - DNA, 105 bp, Simian immunodeficiency virus chimpanzee
tggcagaacaaatgcaggagagatattattagcctatgggaccagagttaaaaccttgtataaagttaaccccattgtgtgttactatgacttgtcttaatcccc

SEQ ID NO: 71 - DNA, 130 bp, Simian immunodeficiency virus chimpanzee
tggcagaacaaatgaatactgacattattagtctgtgggatcaaagtttgaaaccatgtgttaagctgacgccaatatgtgtcaccatggaatgtagaaaggttactttcaatagcacttctaataggaa

SEQ ID NO: 72 - DNA, 130 bp, Simian immunodeficiency virus chimpanzee
tggcagaacaaatgcaggaagatattagtagtctgtgggatcaaagtctaaaaccatgtgttaaactgacaccaatatgtgtcaccatgcaatgtacgacccttaagaacattagcactactaatactaa

SEQ ID NO: 73 - DNA, 125 bp, Simian immunodeficiency virus chimpanzee
  misc_feature (27)..(27): n is unknown
tggtagaaagtatgacagaagatatgntacaattattccaacaaagccataagccatgtgtaaaactaacacctatgtgtataaaaatgaattgtacaggatacaatggaacacctacaacacca

SEQ ID NO: 74 - protein, 35 aa, Human immunodeficiency virus type 1
MVEQMHEDIISLWDQSLKPCVKLTPLCVCLKCTDL

SEQ ID NO: 75 - DNA, 102 bp, Human immunodeficiency virus type 1
aagaatgatactaataccaatagtagtagcgggagaatgataatggagaaaggagagataaaaaactgctctttcaatatcagcacaagcataagaggtaag

SEQ ID NO: 76 - DNA, 84 bp, Human immunodeficiency virus type 1
aacaataatagtactcggaatatcactgaggagtacagaatgacaaactgctctttcaatatgaccacagaactgagagataag

SEQ ID NO: 77 - DNA, 90 bp, Human immunodeficiency virus type 1
gctaatcatactgaggccaataacactgtagaaaacaaagaagaaataaaaaactgctctttcaagataaccacagaaaggggaggcaag

SEQ ID NO: 78 - DNA, 105 bp, Human immunodeficiency virus type 1
aataatagcactgataatagcactgaaaccaacaatagcactgtggataacccaggagaaataaaaaactgctctttcaatgtaaccacagaaataagagataag

SEQ ID NO: 79 - DNA, 87 bp, Human immunodeficiency virus type 1
aatgtaaccacagtagccaacgaaagcgtgagtgcccaagaaataaaaaactgctctttcaatataaccacggaaataagagataga

SEQ ID NO: 80 - DNA, 101 bp, Human immunodeficiency virus type 1
acaaatgtaacaaaaagtaataacagtacagatatcaatataggagagatacaggaacaaagaaactgctctttcaatgtaactacagcataagagataag

SEQ ID NO: 81 - DNA, 108 bp, Human immunodeficiency virus type 1
actagaaatgatactaacagtagcagcactgtcaatgccactagcagtccttcagcaaatgaattaacaaattgctctttcaatgtaactactgtaataagagataag

SEQ ID NO: 82 - DNA, 93 bp, Human immunodeficiency virus type 1
agaaacaatacctctaacagcactagcagtatggaggcaggaggggaactaacaaattgctctttcaatgtaactacagtactaagagataag

SEQ ID NO: 83 - DNA, 102 bp, Human immunodeficiency virus type 1
aatagtaacaatagtacagatagtaatagtagtgcaagcaacaatagtcctgaaatcatgaaaaactgctctttcaatgtaactacagaaataagaaataaa

SEQ ID NO: 84 - DNA, 96 bp, Human immunodeficiency virus type 1
actagtaacagtaatacaactagtaacagtagtgtaagtagtcctgatatcatgacaaactgctctttcaatataactacagaaataagaaataaa

SEQ ID NO: 85 - DNA, 78 bp, Human immunodeficiency virus type 1
aatgcaactagcactgtggtaagcccagcagaaataaaaaattgctctttcaatataaccacagaaataaaagataag

SEQ ID NO: 86 - DNA, 93 bp, Human immunodeficiency virus type 1
aataagaacgataccaatatcaatgcaactgtgacatcaactgacgagataaaaaattgctctttcaatataaccacagaactaaaagataag

SEQ ID NO: 87 - DNA, 69 bp, Human immunodeficiency virus type 1
gctggaacaacaaatgaaaaccttatgaagaagtgtgagtttaatgtaaccactgttatcaaagacaaa

SEQ ID NO: 88 - DNA, 87 bp, Human immunodeficiency virus type 1
caaacaaactaataaggcctattaaatgagacaataaatgagatgagaaattgtagttttaatgtaactacagtcctcacagacaaa

SEQ ID NO: 89 - DNA, 82 bp, Human immunodeficiency virus type 1
ctccataaataatgataccagcagtccagagaaccttgtgaaacagtgtgaatttaatgtaaccacagttgtcaaagacaaa

SEQ ID NO: 90 - DNA, 73 bp, Human immunodeficiency virus type 1
gaatgataccagcagctcagagaaccttatgaaaaagtgtgagtttaatgtaaccacagttctcaaagacaaa

SEQ ID NO: 91 - DNA, 81 bp, Human immunodeficiency virus type 1
tatgacaacaagagaaccagacataggatacaaacaaatgaaaaatgctcattcaatgcaaccactgagctaacagataaa

SEQ ID NO: 92 - DNA, 79 bp, Human immunodeficiency virus type 1
taccaataggacagaggatctagaagacagacaaatgaaaaattgctcattcaatataaccactgagataagagataga

SEQ ID NO: 93 - DNA, 115 bp, Human immunodeficiency virus type 1
  misc_feature (1)..(5): n is unknown
nnnnnannnnnnnnnnnnnnnnnnnnnnnnnnnnnnnnnnnnnaacataataggaaatataacagatgaagtaagaaactgttcttttaatatgaccacagaactaagagataag

SEQ ID NO: 94 - DNA, 102 bp, Human immunodeficiency virus type 1
aatgtgaccaatgtcaataacaatgtcactaacatagtaggaaatataacagaggaagtaagaaactgctcttttaatatgaccacagaactaatagataag

SEQ ID NO: 95 - DNA, 103 bp, Human immunodeficiency virus type 1
accaatggcagtagcaaaaccaatgtctctaacataataggaaatataacagatgaagtaagaaactgtacttttaatatgaccacaagaactaacagataag

SEQ ID NO: 96 - DNA, 91 bp, Human immunodeficiency virus type 1
agttttactaatgccaccagtgacagaataaaaataggaagatgcagtaagaaactgttcttttaatatgaccacagaactacaagataaa

SEQ ID NO: 97 - DNA, 81 bp, Human immunodeficiency virus type 1
acaaccgaagccccaaacatagtaggaacagatgaagtaaagaactgttcttttaatgtgaccacagaactaagagataaa

SEQ ID NO: 98 - DNA, 99 bp, Human immunodeficiency virus type 1
ggccgatgacatgtactaagtctctaacataaccataggaaatataacagatgaagtaagaaactgtacttttaatatgaccacagatcaagtagaaaa

SEQ ID NO: 99 - DNA, 76 bp, Human immunodeficiency virus type 1
agctaagctgaatgacactattggagatgaagtaagaaactgttcttttaatgtgaccacagaactaagggataag

SEQ ID NO: 100 - DNA, 79 bp, Human immunodeficiency virus type 1
caatgtccctaacataggaaatataacagatgaagtaagaaactgttcttttaatatgaccacagaaataagagataag

SEQ ID NO: 101 - DNA, 76 bp, Human immunodeficiency virus type 1
ctctaacataataggaaatataacagatgaagtaagaaactgttcttttaatatgaccacagaactaagagataag

SEQ ID NO: 102 - DNA, 87 bp, Human immunodeficiency virus type 1
aacagaagagccagaaatagaaatatcagaaatgcaaaaagaagtaagcaactgtcttttaatataaccacagaactaagagataag

SEQ ID NO: 103 - DNA, 120 bp, Human immunodeficiency virus type 1
  misc_feature (1)..(12): n is unknown
nnnnnnnnnnnnancagcancannagnancnnnnnnnnnnnnnnnnnnancatatctnntgacatgaaaggggaaataaaaaactgctctttcaatatgaccacagaactaagagataag

SEQ ID NO: 104 - DNA, 75 bp, Human immunodeficiency virus type 1
ggcaacatatcggaaaacatgcaaggagaaataaaaaactgctctttcaatatgaccacagaactaagagataag

SEQ ID NO: 105 - DNA, 72 bp, Human immunodeficiency virus type 1
aacatatctgatggcagcaatagtagaataaaaaactgctcttttaatatgaccacagaattaagagataag

SEQ ID NO: 106 - DNA, 115 bp, Human immunodeficiency virus type 1
agcagcacctctaacagcagcaagcagcacccctatcaacaggaccatagatagtgacatgcaagaggaaataaaaaactgctctttcaatatgaccacagaactaagagataag

SEQ ID NO: 107 - DNA, 99 bp, Human immunodeficiency virus type 1
agcagcaatagcagcatctctgtcaagagcatatctagggacatgcaggaggaaataaaaaattgctctttcaatatgaccacagaactaagagataag

SEQ ID NO: 108 - DNA, 96 bp, Human immunodeficiency virus type 1
aacaacagctacagcgacagtagcaacctaactagtgacatgaatggggaaataaaaaactgctctttcaatataacacaagaagtaagagataag

SEQ ID NO: 109 - DNA, 89 bp, Human immunodeficiency virus type 1
agctactcctgacactactagtaagattaatgaagtggcagaaatgaaaaactgctctttcaatatgaccacagtatataaaagataaa

SEQ ID NO: 110 - DNA, 57 bp, Human immunodeficiency virus type 1
ataaaagaagaaacaaaaaactgctctttcaatatgaccacagacttaagagacaag

SEQ ID NO: 111 - DNA, 105 bp, Human immunodeficiency virus type 1
agtcataactacagcagcatcagtaataacatgactgaggaaatggaaatgaaaggggaaataaaaaactgctctttcaatatgaccactgaactaagagataag

SEQ ID NO: 112 - DNA, 88 bp, Human immunodeficiency virus type 1
aagacagaatgatactgataccaatgcgtagtggcataggagatgatgaaaaactgctctttcaactcacacaagacctggagataaa

SEQ ID NO: 113 - DNA, 93 bp, Human immunodeficiency virus type 1
aagaaggaggttactagtaccaatacgtagtagcataaaagtgatggaaatgaaaaactgctctttcaatctcacacaagacctggagataaa

SEQ ID NO: 114 - DNA, 94 bp, Human immunodeficiency virus type 1
accattaaaagtactaatagtacccccaatgtaacaaccacaaacgaagagaaaaactgctctttcaatataaccacagaaaataagagataag

SEQ ID NO: 115 - DNA, 104 bp, Human immunodeficiency virus type 1
agtactaacaatagctaccctaatagtactggcaatagcactgagaagagtacggcgagataaaaaactgctctttcaatataaccacagaagtaagagataag

SEQ ID NO: 116 - DNA, 87 bp, Human immunodeficiency virus type 1
actactactaatagtaccaatggcactgtgaataaagaaggaataaaaaactgctctttcgatataaccacagaaataagggataag

SEQ ID NO: 117 - DNA, 88 bp, Human immunodeficiency virus type 1
ttccaccaccagctccactaccctgaaagaagagacaggggcagtacaaaactgttctttcaatatgactacagaagtaaatgacaaa

SEQ ID NO: 118 - DNA, 125 bp, Human immunodeficiency virus type 1
tgccaccaccactagcaagaacatcagtgccacccccactagcaaccccaatgacaccctgaaggaagagcaaggggcaatacaaaactgctatttcaatataaccacaagaagtaaaggataag

SEQ ID NO: 119 - DNA, 92 bp, Human immunodeficiency virus type 1
acagatcatggaataaacaacaataacactgtagagggtaaagaagaataaaaaactgctcttttaatgtaaccacagaaataagagataag

SEQ ID NO: 120 - DNA, 111 bp, Human immunodeficiency virus type 1
aggtaacaatagctcatatcgatcattaggtaacaatagtactatatagatgatatcagcaaggaaataaaaaactgctctttcaatataaccacagaaataagagataag

SEQ ID NO: 121 - DNA, 73 bp, Human immunodeficiency virus type 1
aaatatcactgtggagagcggagaagaaataaaaaactgctctttcaatgtaaccacagaaataagagataag

SEQ ID NO: 122 - DNA, 81 bp, Human immunodeficiency virus type 1
tgtgggtaaaagtttaactgtgaaggacagagagaaataaaaaactgctctttcaatataaccacagaagtaagagatcag

SEQ ID NO: 123 - DNA, 52 bp, Human immunodeficiency virus type 1
ccccagaatgacaaactgctctttcaatacaaccacagaagtaaaagataag

SEQ ID NO: 124 - DNA, 67 bp, Human immunodeficiency virus type 1
tgtcaatgccactaacaatgtcggatgaaaactgctctttcaatataaccacagaataagagataag

SEQ ID NO: 125 - DNA, 73 bp, Human immunodeficiency virus type 1
cactaacagcacagatagtgaaccgggaatgaaaaactgctctttcaatataaccacagaaataagagataag

SEQ ID NO: 126 - DNA, 48 bp, Human immunodeficiency virus type 1
gaaatgaaaaactgctctttcaatgtaaccacagaactaatagatagg

SEQ ID NO: 127 - DNA, 84 bp, Human immunodeficiency virus type 1
agcagatgtaaccaacagcatgatagaagaaaagggagaaatagaaactgctctttcagggtaaccacagaggaagcatataaa

SEQ ID NO: 128 - DNA, 84 bp, Human immunodeficiency virus type 1
gatacaaccgaagttgataggcaagacgacatgttgaatggcaccttaagtgtgctcgaaaggtaactggaagaaaatacacat

SEQ ID NO: 129 - DNA, 51 bp, Human immunodeficiency virus type 1
acagacataaaaaactgcactttcaatataaccacagaactagaagataag

SEQ ID NO: 130 - DNA, 88 bp, Simian immunodeficiency virus chimpanzee
caggcaaaatacctataaaaccagtcatctttctccgcctctcgaatagaaaactgttcatttaatgtgacaacagaattaagataag

SEQ ID NO: 131 - DNA, 83 bp, Simian immunodeficiency virus chimpanzee
gatagacatagtagaggctgtaaatactactgatataatgagaaactgttctttctatacaagctcatgaaataagagacaaa

SEQ ID NO: 132 - DNA, 108 bp, Simian immunodeficiency virus chimpanzee
taaaacttctacattacttctacattgacatatccctagcctaatgataaaayagacagcacagtaaagaattgtacctttaatatgactactgaggtaagagataaa

SEQ ID NO: 133 - DNA, 76 bp, Simian immunodeficiency virus chimpanzee
cagtacagagttaaaaaatataaaagaaataatgaaaaattgtacatttaatatgactactgaagtaagagataaa

SEQ ID NO: 134 - DNA, 107 bp, Simian immunodeficiency virus chimpanzee
agtacaacaacaagaccagtaacaccaaagacaacaacaccaatagtagatggcatgaagctacaagaagctaactttatacagagcacagaggatttaaagataag

SEQ ID NO: 135 - protein, 34 aa, Human immunodeficiency virus type 1
KNDTNTNSSSGRMIMEKGEIKNCSFNISTSIRGK

SEQ ID NO: 136 - protein, 15 aa, Human immunodeficiency virus type 1
NLNITKNTTNPTSSS

SEQ ID NO: 137 - protein, 15 aa, Human immunodeficiency virus type 1
TKNTTNPTSSSWGMM

SEQ ID NO: 138 - protein, 15 aa, Human immunodeficiency virus type 1
TNPTSSSWGMMEKGE

SEQ ID NO: 139 - protein, 15 aa, Human immunodeficiency virus type 1
SSSWGMMEKGEIKNC

SEQ ID NO: 140 - protein, 15 aa, Human immunodeficiency virus type 1
GMMEKGEIKNCSFYI

(19) United States
(12) Patent Application Publication
(10) Pub. No.: US 2006/0205070 A1
(43) Pub. Date: Sep. 14, 2006

(54) HIV TEV compositions and methods of use

(75) Inventors: Miroslav Dundr, Bethesda, MD (US); Genoveffa Franchini, Washington, DC (US); Jake R. Fullen, Upper Marlboro, MD (US)

Correspondence Address: MERCHANT & GOULD PC, P.O. BOX 2903, MINNEAPOLIS, MN, 55402-0903, US

(73) Assignee: The Government of the USA, as represented by the Secretary, Department of Health and Human Services, Rockville, MD (US)

(21) Appl. No.: 11/364,873

(22) Filed: Feb. 27, 2006

Related U.S. Application Data
(63) Continuation-in-part of application No. 11/165,444, filed on Jun. 23, 2005, now abandoned.
Application No. 11/165,444 is a continuation of application No. PCT/US05/01214, filed on Jan. 13, 2005.
(60) Provisional application No. 60/536,467, filed on Jan. 13, 2004.
(60) Provisional application No. 60/536,976, filed on Jan. 16, 2004.

(51) Int. Cl. C12Q 1/68; C12N 5/06; C12N 5/16

(52) U.S. Cl. 435/345; 435/6

(57) ABSTRACT

The disclosure encompasses p28^{TEV} polypeptide, polynucleotides, variants, and antagonists. p28^{TEV} polypeptides and/or polynucleotides are useful in immunogenic compositions. p28^{TEV} polypeptide antagonists include antagonist antibodies, antisense molecules or siRNA molecules. The antagonists and composition of the disclosure can be administered alone or in combination with other agents useful in the treatment of HIV infection, SIV infection, AIDS, or AIDS-related complex (ARC), including nucleoside, non-nucleoside, and/or reverse transcriptase inhibitors.

### STATEMENT OF RIGHTS TO DISCLOSURES MADE UNDER FEDERALLY SPONSORED RESEARCH AND DEVELOPMENT

The work performed during the development of this disclosure utilized intramural support from the National Institutes of Health. The United States government has certain rights in the disclosure.

### FIELD OF THE DISCLOSURE

This disclosure concerns human immunodeficiency virus (HIV) p28^{TEV} protein and antagonists and methods of use, including methods of inhibiting HIV viral levels.

### BACKGROUND OF THE DISCLOSURE

It is estimated that approximately 14,000 people worldwide are infected every day with human immunodeficiency virus (HIV). Many advances have been made in treating HIV infection and related Acquired Immune Deficiency Syndrome (AIDS), however, components of HIV that provide long lasting and effective immune responses remain to be identified. Unfortunately, HIV mutates rapidly resulting in multiple types and subtypes of the virus even in a single individual. Understanding how HIV infection operates in humans is an important component in developing effective therapies.

HIV type 1 (HIV-1) infects human CD4+ cells and persists despite a strong host immune response. The viral genome is reverse transcribed into a provirus that is integrated into the host genome. Butera, 2000, Antiviral Res., 48:143-176. In individuals undergoing highly active anti-retroviral therapy (HAART, regimens including various combinations of nucleoside, non-nucleoside, and protease inhibitors), levels of detectable virus remain for extended periods of time. Butera, 2000, Antiviral Res., 48:143-176.

Tat and Rev are virally encoded regulatory factors for HIV gene expression. Tat acts by binding to the TAR RNA element and activating transcription initiation and/or elongation from the LTR promoter. Defects in the coding region of Tat or mutations that disrupt binding to TAR restrict HIV-1 transcriptional activity. S.T. Butera, 2000, Antiviral Res., 48:143-176. Rev is localized in the nucleolus/nucleus and cytoplasm. Rev interacts with Rev response element (RRE) in HIV-1 mRNA to protect full-length messages from nuclear RNA splicosomes and allows transport into the cytoplasm. Human Retroviruses and AIDS 1996, Eds. Myers et al., Los Alamos National Laboratory, 1996; S.T. Butera, 2000, Antiviral Res., 48:143-176. Defects in the coding region of Rev or mutations that disrupt binding to RRE also restrict HIV-1 transcriptional activity.

HIV-1 also encodes alternatively spliced mRNAs for several proteins involved in the regulation of transcription. In addition to Tat and Rev described above, two mRNAs encoding two proteins, p18 and p28^{TEV}, have also been described. Benko et al., 1990, J. Virol., 64:2505-2518. HIV-1 p28^{TEV} is encoded by alternatively spliced mRNA that joins together the first exon of Tat to a region with the Env (exon 6D) and to the second exon of Rev. The exon of the Env gene spliced with p28^{TEV} includes the VI region of the virus. HIV-1 p18 is produced by splicing exon 6D to the second exon of Rev. HIV-1 p2₈TEV exhibits both Tat and Rev function and can functionally replace both regulatory proteins in HIV-1. The function of the p28^{TEV} and p18 in regulating viral expression in infected cells is not known. Human Retroviruses and AIDS 1996, Eds. Myers et al., Los Alamos National Laboratory, 1996 (accessible on the internet at http://hiv-web.lanl.gov).

A great deal of effort is being directed to the design and testing of agents that inhibit HIV infectivity or prevent HIV infection. Effective inhibitors of HIV infectivity are still needed.

### SUMMARY

One aspect of the disclosure is an immunogenic composition comprising at least one isolated nucleic acid encoding at least one p28^{TEV} polypeptide, preferably, a p28^{TEV} polypeptide having at least about 70 percent amino acid sequence identity with an amino acid sequence selected from the group consisting of SEQ ID NO:151, SEQ ID NO:143, SEQ ID NO:146, SEQ ID NO:1 and fragments thereof. In some embodiments the p28^{TEV} polypeptide retains transcriptional activation activity and/or the V1 region. In some embodiments, the composition may comprise one or more isolated nucleic acids encoding one or more p28^{TEV} polypeptides from one or more HIV-1 clades. In some embodiments, the composition may comprise one or more isolated nucleic acids encoding one or more p28^{TEV} polypeptides selected from the group consisting of polypeptides comprising an amino acid sequence of SEQ ID NO: 143, SEQ ID NO:146, SEQ ID NO:1, and SEQ ID NO: 151 and fragments thereof. The fragments preferably include the V1 region. In other embodiments, the fragment comprises an amino acid sequence selected from the group consisting of SEQ ID NO:137, SEQ ID NO:138, SEQ ID NO:139, SEQ ID NO:140, SEQ ID NO:152, SEQ ID NO:153, SEQ ID NO:154, SEQ ID NO:155, SEQ ID NO:156, SEQ ID NO:164, SEQ ID NO:165, SEQ ID NO:166, SEQ ID NO:167, SEQ ID NO:168, and SEQ ID NO 169.

The composition may also comprise an immunomodulator, for example, an adjuvant. In some embodiments, the composition reduces or inhibits HIV viral levels upon administration to a mammal. Methods of using the immunogenic composition to immunize animals and reduce HIV viral levels are also provided.

Another aspect of the disclosure is a composition comprising a physiologically acceptable carrier and an effective amount of at least one isolated and /or purified p28^{TEV} polypeptide or an immunogenic fragment thereof. Preferably, the composition when administered reduces or inhibits viral levels. The composition may comprise p28^{TEV} polypeptides from one or more HIV-1 clades. In some embodiments, the polypeptide comprises an amino acid sequence having at least about 70 percent and up to 100% sequence identity amino acid identity with a reference p28^{TEV}, including any number of % sequence identity between 70% and 100% sequence identity. In some embodiments, the polypeptide has at least about 80 percent amino acid identity, has at least about 90 percent amino acid identity, or at least about 95% sequence identity with a reference p28^{TEV} polypeptide. In some embodiments, one or more of the p28^{TEV} polypeptides have the sequence of naturally occurring polypeptides from one or more HIV clades. In still another embodiment, the polypeptide is a fragment of a p28^{TEV} polypeptide comprising all or portion of the V1 region, preferably corresponding to amino acids 75-103 of p28^{TEV} of isolate 89.7. In still another embodiment, the reference polypeptide comprises an amino acid sequence selected from the group consisting of SEQ ID NO:1, SEQ ID NO:143, SEQ ID NO:146, SEQ ID NO:151, and fragments thereof. In other embodiments, the referencing polypeptide comprises an amino acid sequence selected from the group consisting of SEQ ID NO:137, SEQ ID NO:138, SEQ ID NO:139, SEQ ID NO:140, SEQ ID NO:152, SEQ ID NO:153, SEQ ID NO:154, SEQ ID NO:155, SEQ ID NO:156, SEQ ID NO:164, SEQ ID NO:165, SEQ ID NO:166, SEQ ID NO:167, SEQ ID NO:168, and SEQ ID NO 169. The composition may also comprise an immunomodulator, for example, an adjuvant.

Another aspect of the disclosure is directed to methods of using an immunogenic composition to reduce or inhibit HIV viral levels. In some embodiments, a method comprises administering an immunogenic composition to a mammal comprising one or more isolated nucleic acids encoding a p28^{TEV} polypeptide. In some embodiments, the immunogenic composition comprises at least one isolated and/or purified p28^{TEV} polypeptide and, more preferably two or more p28^{TEV} polypeptides, each p28^{TEV} polypeptide from a different HIV clade. In some embodiments, the composition further comprises an immunomodulator or adjuvant. In some embodiments, the composition is administered to a human.

Another aspect is directed to an antibody that specifically binds a p28^{TEV} polypeptide and an article of manufacture comprising the antibody. In some embodiments, the antibody inhibits or reduces HIV-1 levels. In some embodiments, the antibody inhibits p28^{TEV} activity. In some embodiments, the polypeptide comprises an isolated p28^{TEV} polypeptide comprising a sequence of SEQ ID NO:151, SEQ ID NO:143, SEQ ID NO:146, SEQ ID NO:1 or fragments thereof. The fragment preferably includes the V1 region. In other embodiments, the polypeptide comprises an amino acid sequence having at least about 70 percent and up to 100% sequence identity amino acid identity with a reference p28^{TEV}, including any number of % sequence identity between 70% and 100% sequence identity. In some embodiments, the polypeptide has at least about 80 percent amino acid identity, has at least about 90 percent amino acid identity, or at least about 95% sequence identity with a reference p28^{TEV} polypeptide. In some embodiments, one or more of the p28^{TEV} polypeptides have the sequence of naturally occurring polypeptides from one or more HIV clades. In still another embodiment, the polypeptide or reference polypeptide is selected from the group consisting a polypeptide comprising the amino acid sequence of SEQ ID NO:1, SEQ ID NO:143, SEQ ID NO:146, SEQ ID NO:151, SEQ ID NO:137, SEQ ID NO:138, SEQ ID NO:139, SEQ ID NO:140, SEQ ID NO:152, SEQ ID NO:153, SEQ ID NO:154, SEQ ID NO:155, and SEQ ID NO:156. In some embodiments, an antibody that specifically binds a p28^{TEV} polypeptide is generated by or binds to a consensus sequence of V1 from different clades comprising an amino acid sequence selected from the group consisting of SEQ ID NO:164, SEQ ID NO:165, SEQ ID No:166, SEQ ID NO:167, SEQ ID NO:168, and SEQ ID NO:169. In some embodiments, the antibody specifically binds to the V1 region and does not specifically or significantly bind to the tat or rev region. The antibody may be a monoclonal antibody or a humanized antibody.

The article of manufacture comprises a container, an agent contained within the container, and a label, wherein the agent comprises at least one antibody or antigen binding fragment that binds specifically to a p28^{TEV} polypeptide. The antibodies may be any combination of antibodies that specifically bind V1 in combination with anti-tat or anti-rev or both antibodies. The antibody or antigen binding fragment may be attached to a solid substrate. The solid substrate may be any suitable solid substrate, including the container or a multi-well plate. The antibody can be attached to a cytotoxic agent, siRNA molecule, or other inhibitor of HIV. The antibody or antigen binding fragment may be detectably labeled. The label may be a fluorescent moiety, radioactive moiety, peptide tag, or an enzyme.

The antibodies are also useful, for example, in methods for reducing viral levels or detecting virus in a host. In an embodiment, the method comprises a) contacting a biological sample with at least one antibody that specifically binds a p28^{TEV} polypeptide and b) detecting the presence of p28^{TEV} in the biological sample.

Another aspect of the disclosure is an antagonist of p28^{TEV} comprising an isolated polynucleotide that inhibits expression of p28^{TEV}, vectors comprising the antagonist, and host cells comprising the vector. The polynucleotide may inhibit expression directly or encode a molecule that inhibits expression of p28^{TEV}. In an embodiment, the polynucleotide is an antisense RNA molecule. In another embodiment, the polynucleotide encodes an antisense RNA molecule. In another embodiment, the polynucleotide is a small interfering RNA (siRNA) molecule. In still another embodiment, the polynucleotide encodes a siRNA molecule.

In some embodiments, the polynucleotide hybridizes under stringent conditions to a nucleic acid molecule encoding p28^{TEV} or a p28^{TEV} splice acceptor site and/or a p28^{TEV} splice donor site. In an embodiment, the polynucleotide comprises a polynucleotide sequence of SEQ ID NO:2. The stringent conditions may be stringent, moderately stringent, or highly stringent. In an embodiment, the nucleic acid molecule encoding p28^{TEV} comprises a polynucleotide sequence encoding an ENV exon. In a preferred embodiment, the exon is exon 6D. In an embodiment, the nucleic acid molecule encoding a p28^{TEV} splice acceptor site comprises a polynucleotide sequence of SEQ ID NO:3. In another embodiment, the nucleic acid molecule encoding the p28^{TEV} splice acceptor site comprises a splice acceptor site at nucleotide 6154 of mRNA of HXB2. In an embodiment, the nucleic acid molecule encoding a p28^{TEV} splice donor site comprises a polynucleotide sequence of SEQ ID NO:4. In another embodiment, the nucleic acid molecule encoding the p28^{TEV} splice donor site comprises a splice acceptor site at nucleotide 6269 of mRNA of HXB2.

Antagonists comprising a polynucleotide that inhibits expression of p28^{TEV} are useful, for example, in methods for reducing viral levels. In an embodiment, the method comprises administering to a patient in need thereof an effective amount of the antagonist to inhibit HIV-1 viral levels.

The present disclosure is also directed to methods for identifying antagonists of p28^{TEV} polypeptide. In an embodiment, the method comprising contacting a p28^{TEV} polypeptide or cell comprising a polynucleotide encoding a p28^{TEV} polypeptide with a candidate agent, and determining whether the candidate agent inhibits an activity or expression of p28^{TEV} polypeptide. A candidate agent that inhibits the activity or expression of p28^{TEV} polypeptide is identified as an antagonist of the p28^{TEV} polypeptide. The antagonist may reduce HIV viral levels and/or transcriptional activity of p28^{TEV}.

Another aspect of the disclosure is a method for inhibiting HIV viral levels. In an embodiment, the method comprises administering to a patient in need thereof an effective amount of an antagonist. In some embodiments, the antagonist is administered as a therapeutic agent to a patient infected with HIV, either alone or in combination with other antiviral therapies. In a preferred embodiment, the antagonist is an antibody. The antibody may be monoclonal. In an embodiment, the monoclonal antibody is humanized. In another embodiment, the monoclonal antibody is a Fv, Fab, Fab′, or F(ab′)₂ fragment. In another preferred embodiment, the antagonist is a polynucleotide as described above.

Another aspect of the disclosure is a method for inhibiting or reducing HIV viral levels. In an embodiment, the method comprises administering an effective amount of an immunogenic composition to a mammal. In some embodiments, the immunogenic composition comprises one or more isolated nucleic acids encoding p28^{TEV}, optionally in combination with an immunomodulator. In other embodiments, the immunogenic composition comprises one or more isolated and purified p28^{TEV} polypeptides, preferably from different HIV clades, optionally in combination with an immunomodulator. In some embodiments, the immunogenic composition comprises one or V1 peptides optionally in combination with an immunomodulator.

The antagonists or immunogenic compositions of the disclosure can be administered in combination with other agents useful in the treatment of HIV infection, SIV infection, AIDS, or AIDS-related complex (ARC), including nucleoside, non-nucleoside, and/or reverse transcriptase inhibitors.

### BRIEF DESCRIPTION OF THE FIGURES

FIG. 1 shows that p28^{TEV} is expressed in the nucleolus and cytoplasm of HeLa cells transfected with a vector encoding p28^{TEV} fused to GFP at the C terminus. HTLV-1 p30¹¹ protein fused at the C terminus to GFP was used as a control and is expressed in the nucleolus only.

FIG. 2A shows the efficiency of transfection of 293T cells in each experimental condition using the RLTK construct. Cells were co-transfected with vectors encoding pHXB2 (Fisher et al., 1986, Science, 233:655-659); p28^{TEV} ; RL-TK (Promega Corp., Madison, Wis.); and HIV-Luc (M. R. Smith and W. C. Greene, 1990, Genes Dev., 4:1875-1885). The cells were transfected with increasing amounts of the vector encoding HXB2 p28^{TEV} from 0.5 to 10 ug. Transfection efficiency was determined by measuring expression levels of Renilla luciferase. The results show equivalent RLTK expression in all the experimental conditions.

FIG. 2B shows that increasing amounts of HXB2 p28^{TEV} expression increased luciferase from the HWV-Luc reporter gene. The HIV-Luc reporter gene was constructed using the 5′ LTR region of HIV-1 strain fused to the firefly luciferase reporter gene. p28^{TEV} contains the Tat-activating domain. The results show stimulation of transcription activity of HIV-1 LTR in the presence of increasing amounts of trans HXB2 p28^{TEV}

FIG. 2C shows that even though the Tat activity increased in the presence of increasing amounts of trans p28^{TEV}, viral production as measured by p24 in the supernatant decreased.

FIG. 2D shows a western blot of HIV-1 p24. The results show that intracellular expression of HIV-1 p24 was decreased with increasing amounts of expression of p28^{TEV} whereas the level of α-tubulin, a housekeeping protein, remained constant. p28^{TEV} was also detected in the supernatant of the transfected cells. Increasing amounts of p28^{TEV} were added to lanes starting from left to right.

FIGS. 3A shows the increase in expression of HIV-Luc in the presence of increasing amounts of trans HXB2 p28^{TEV} and no HIV-Luc activity in the absence of the HIV-Luc reporter gene (last three lanes).

FIG. 3B shows a dose-dependent decrease of p24 in the supernatant in the presence of increasing amounts of HXB2 p28^{TEV} both in the presence and absence of HIV-Luc indicating that exogenous HIV-1-Luc is not competing for the transactivation of Tat produced by the provirus.

FIG. 4 shows a representative amino acid sequence of HIV-1 p₂₈,rEv (SEQ ID NO:1) from HXB2.

FIG. 5 shows a representative nucleic acid sequence of HIV-1 p28^{TEV} (SEQ ID NO:2) from HXB2.

FIG. 6 shows the position of the splice acceptor site and splice donor site in a nucleic acid construct encoding p28^{TEV}.

FIG. 7 shows an alignment of DNA sequences of representative HIV-1 isolates wherein the reference sequence is HXB2. Other similar sequences can be found at the Los Alamos database. The location of the splice acceptor site for p28^{TEV} in mRNA of HXB2 is identified. (SEQ ID NOs:14-74)

FIG. 8 shows an alignment of DNA sequences of representative HIV-1 isolates wherein the reference sequence is HXB2. Other similar sequences can be found at the Los Alamos database. The location of the splice donor site for p28^{TEV} in mRNA of HXB2 is identified. (SEQ ID NOs:75-135)

FIG. 9. Top: schematic representation of the 3′ ORF that participates in the generation of Tev. Middle: DNA sequence of the primer used to mutagenize the 6 nucleotides within the acceptor splice site for Tev in the pHXB2, pNL4, and 89.6 mutant clones (bottom). The sequence of the primer is the following:

5′-CTCTGTGTTTCACTGAAGTGCACT-3′ (SEQ ID NO:11)

FIG. 10 293T cell were transfected with 10 μg each of pHXB2 or pHXB2ΔTev. p24 levels were measured on the supernatant at 24 and 48 h (top panel). Western blots of the cell lysates are shown in the bottom panels. Rev expression was augmented in ΔTev clones. The expression and cleavage of the Env proteins was not affected. This result was expected as the Env precursor is cleaved by cellular protease.

FIG. 11: Panel A: 293T cell cultures were separately transfected with 10 μg each of pME, pHXB2, pHXB2ΔTev, pNL, pNL4-3ΔTev, 89.6, and 89.6ΔTev. p24 levels were measured on the supernatant at 48 h.
Panel B: Western blot of cell lysates using antibodies to gp160 and CD71
Panel C: 293T cells were separately transfected with 10 μg each of pME, pNL4-3, and increasing amounts of cDNA encoding Rev and p24 production was measured.
Panel D: TZM cells were exposed to equivalent amounts of p24 from the supernatant of cells transfected with pME, pHXB2, pHXB2ΔTev, pNL, pNL4-3ΔTev, 89.6, and 89.6ΔTev. The cells were lysed at 48 h and luciferase activity measured.
Panel E: TZM cell cultures exposed separately to the supernatant from pME, pHXB2, pHXB2ΔTev, pNL, pNL4-3ΔTev, 89.6, and 89.6ΔTev. Transfected cells were lysed at 48 h and reverse transcriptase activity measured.

FIG. 12: (A) Expression of cDNAs of recombinant Tev protein from BaL, pHXB2, SF162, and 89.6P HIV clones in E. coli cells. (B) Electrophoretic analysis of the purified 89.6P protein detected by Coomasie blue staining (Lane 1) and molecular weight markers (Lane 2). (C) Schematic representation of the study design and immunization regimen. Animals in group 1 received three immunizations with 4 mg each of plasmid encoding BaL, SF162, and 89.6P Tev proteins by the intramuscular route and boosted with 200 μg of 89.6P Tev protein together with 1 mg of CpG class B at the time indicated. Intravenous challenge with highly pathogenic chimeric SHIV89.6P was performed at week 22.

FIG. 13A shows viral loads of animals immunized with a representative immunogenic composition. FIG. 13B shows viral loads of control animals that were not immunized.

FIG. 14A shows CD3+/CD4+ T-cell counts of immunized animals. FIG. 14B shows CD3+CD4+T-cell counts of control animals.

FIG. 15 shows ELISPOT responses in control and vaccinated animals to (A)BaL tev, (B)SF162tev, and (C)89.6 Tev in animals after challenge with SHIV89.6P was performed at week 22 (day of challenge) and thereafter; Panel D shows the response of T cells to Con A as a control of cell viability.

FIG. 16 shows antibody titers to HIV-1 IIIB Tat antibodies in animals immunized with a representative immunogenic composition and control animals before and after challenge with SHIV89.6P.

FIG. 17 shows antibody titers to HIV-1 MN Rev antibodies in animals immunized with a representative immunogenic composition and control animals before and after challenge with SHIV89.6P.

FIG. 18 shows antibody titers to HIV-1 Env V1 antibodies in animals immunized with a representative immunogenic composition and control animals before and after challenge with SHIV89.6P. The peptides used in this assay were

| BAL: | CRNATNGNDTNTTSSR | (SEQ ID NO:157) |
|---|---|---|
| SF162: | KNATNTKSSNWKEMDRC | (SEQ ID NO:158) |
| 89.6: | CKNTNTPTSSSWGMMEK | (SEQ ID NO:159) |

FIG. 19 ELISA Ab response to overlapping peptides from 89.6P Tev proteins on immunized animals 316 and 308 at week 10, week 19, week 24 (post challenge with SHIV89.6 at week 22), and week 27. An increase in antibodies to peptides 21-24 corresponding to V1 is seen and is outlined.

FIG. 20 ELISA Ab response to overlapping peptides from 89.6P Tev proteins on immunized animals 490 and 218 at week 10, week 19, week 24 (post challenge with SHIV89.6 at week 22), and week 27. An increase in antibodies to peptides 21-24 corresponding to V1 is seen in 490 but not in 218 and is outlined.

FIG. 21 ELISA Ab response to overlapping peptides from 89.6P Tev proteins on immunized animal 316 at week 10, week 19, week 24 (post challenge with SHIV89.6 at week 22), and week 27. An increase in antibodies to peptides 21-24 corresponding to V1 is seen even at 1:5,000 serum and is outlined.

FIG. 22 ELISA Ab response to overlapping peptides from 89.6P Tev proteins on control animals 915 and 320 at week 10, week 19, week 24 (post challenge with SHIV89.6 at week 22), and week 27. No increase in antibodies to peptides 21-24 corresponding to V1 is seen.

FIG. 23 (A) ELISA Ab response to overlapping peptides from 89.6P Tev proteins on immunized animals 316, 308, 218, 490 and control animals 915 and 320 at week 10, week 19, week 24 (post challenge with SHIV89.6 at week 22), and week 27. An increase in antibodies to peptides 21-24 of 89.6 Tev corresponding to V1 is seen post challenge in the immunized animals; (B) ELISA response to overlapping peptides from BaL Tev proteins on immunized animals 316, 308, 218, 490 and control animals 915 and 320 at week 10, week 19, week 24 (post challenge with SHIV89.6 at week 22), and week 27. No cross-reactivity of antibodies to peptides 21-24 corresponding to BaL Tev V1 is seen; (C) ELISA response to overlapping peptides from SF162 Tev proteins on immunized animals 316, 308, 218, 490 and control animals 915 and 320 at week 10, week 19, week 24 (post challenge with SHIV89.6 at week 22), and week 27. No cross-reactivity of antibodies to peptides 21-24 corresponding to SF162 Tev V1 is seen.

FIG. 24 shows the SIV RNA load in different tissues: lymph nodes, jejunum, spleen, and plasma, at the time of sacrifice (6 months from challenge exposure). Animals M320 and 915L are control nonimmunized animals.

FIG. 25 shows the results of antibody dependent cellular cytotoxicity of plasma samples from immunized and nonimmunized animals at week 22, week 26 and week 28. All animals were challenged at week 22 with SHIV89.6. Animals 915 and 320 are nonimmunized control animals.

FIG. 26 shows the polynucleotide sequence encoding tev from the BaL HIV isolate. (SEQ ID NO:141) This sequence is codon optimized for expression in humans and E. coli. The lower sequence is a synthetic gene sequence prepared as described in the examples, sequenced and compared to the sequences provided (top sequence).

FIG. 27 shows the polynucleotide(SEQ ID NO:142) and amino acid sequence for tev(SEQ ID NO:143) from the BaL HIV isolate from plasmid pPCR-Script Amp.

FIG. 28 shows the polynucleotide sequence encoding tev from the SF162 HIV isolate. (SEQ ID NO:144) This sequence is codon optimized for expression in humans and E. coli. The lower sequence is a synthetic gene sequence prepared as described in the examples, sequenced and compared to the sequences provided (top sequence).

FIG. 29 shows the polynucleotide(SEQ ID NO:145) and amino acid sequence for tev (SEQ ID NO:146) from the SF162 HIV isolate from plasmid pPCR-Script Amp.

FIG. 30 shows a polynucleotide sequence encoding tev from the HXB2 HIV isolate. (SEQ ID NO:147) This sequence is codon optimized for expression in humans and E. coli. The lower sequence is a synthetic gene sequence prepared as described in the examples, sequenced and compared to the sequences provided (top sequence).

FIG. 31 shows a polynucleotide(SEQ ID NO:148) and amino acid sequence for tev (SEQ ID NO:1) from the HXB2 HIV isolate from plasmid pPCR-Script Amp.

FIG. 32 shows a polynucleotide sequence encoding tev from the 89.6 HIV isolate. (SEQ ID NO:149) This sequence is codon optimized for expression in humans and E. coli. The lower sequence is a synthetic gene sequence prepared as described in the examples, sequenced and compared to the sequences provided (top sequence).

FIG. 33 shows the polynucleotide(SEQ ID NO:150) and amino acid sequence (SEQ ID NO:151) for tev from the 89.6 HIV isolate from plasmid pPCR-Script Amp.

FIG. 34 shows the plasmid maps for each plasmid that has a polynucleotide encoding tev. Panel A is the plasmid map for BaL tev. Panel B is the plasmid map for SF162 tev. Panel C is the plasmid map for HXB2 tev. Panel D is the plamid map for 89.6 tev.

FIGS. 35A and B show an alignment of V1 sequences from several different HIV isolates or strains obtained from the Los Alamos database. The consensus sequences for the V1 region of each clade can be identified by comparing the sequences between the two cysteines identified by the first two arrows.

### DETAILED DESCRIPTION

I. Definitions The terms “p28^{TEV} polypeptide” or “p281EV protein” or “p28^{TEV}” or “p28” are used interchangeably and encompass both naturally occurring p28^{TEV} polypeptides or proteins and p28^{TEV} polypeptide variants. In an embodiment, p28_{TEV} polypeptide comprises an amino acid sequence of SEQ ID NO:151 (FIG. 33). In other embodiments, p28^{TEV} polypeptide comprises an amino acid sequence selected from the group consisting of SEQ ID NO:1, SEQ ID NO:143, SEQ ID NO:146, and SEQ ID NO:151.

The terms “naturally occurring p28^{TEV} polypeptide” or “naturally occurring p28^{TEV}” are used interchangeably and encompass polypeptides that have the same amino acid sequence of a polypeptide obtained from nature from HIV or an HIV infected cell. The terms “naturally occurring p28^{TEV} polypeptide” or “naturally occurring p28^{TEV}” specifically encompasses any of the naturally occurring forms of the polypeptides including polypeptides with a signal sequence and/or mature forms without the signal sequence. Naturally occurring variants include secreted forms, alternatively spliced forms, and those naturally occurring variants from other HIV-1 strains or isolates that differ in sequence from a reference sequence for a particular p28^{TEV} polypeptide. In an embodiment, the reference sequence comprises an amino acid sequence of SEQ ID NO:151. Naturally occurring p28^{TEV} polypeptides are expressed early in infection and have a biological activity of transcriptional activation. Naturally occurring p28^{TEV} polypeptides or proteins can be isolated or purified from nature, prepared recombinantly or synthetically.

“p28^{TEV} polypeptide variant” or “p28^{TEV} protein variant” or “p28^{TEV} variant” refers to a p28^{TEV} polypeptide that differs in amino acid sequence from a particular p28^{TEV} polypeptide reference sequence. In an embodiment, the p28^{TEV} polypeptide reference sequence comprises an amino acid sequence of SEQ ID NO:151. “p28^{TEV} variant polypeptides” or “p28^{TEV} variant proteins” or “p28^{TEV} variants” specifically encompasses modifications of the reference sequence, and naturally occurring p28^{TEV} polypeptide variants. When the variant is a naturally occurring p28^{TEV} polypeptide variant of the reference sequence, the variant is designated “a naturally occurring p28^{TEV} variant.” The variants may include deletions and additions of amino acids, as well as amino acid substitutions.

A p28^{TEV} variant has at least about any number of % sequence identity from 70% to 100% sequence identity to a full-length mature p28^{TEV} polypeptide reference sequence. A p28^{TEV} variant has at least about 70% sequence identity, more preferably at least about 75% sequence identity, more preferably at least about 80% sequence identity, more preferably at least about 85% sequence identity, more preferably at least about 90% sequence identity, more preferably at least about 95% sequence identity and even 100% sequence identity to a full-length mature p28^{TEV} polypeptide reference sequence, such as a polypeptide having the sequence of SEQ ID NO:151. In other embodiments, a p28^{TEV} reference polypeptide comprises an amino acid sequence selected from the group consisting of SEQ ID NO:1, SEQ ID NO:143, SEQ ID NO:146, SEQ ID NO:151, SEQ ID NO:137, SEQ ID NO:138, SEQ ID NO:139, SEQ ID NO: 140, SEQ ID NO: 152, SEQ ID NO: 153, SEQ ID NO: 154, SEQ ID NO: 155, and SEQ ID NO: 156. In some embodiments, the polypeptide variants increase Tat transcriptional activity in the HlV-1-Luc assay at least 2 fold.

The term “isolated,” when used to describe the various polypeptides disclosed herein, means a polypeptide that has been identified and separated and/or recovered from a component of its natural environment. Preferably, the isolated polypeptide is free of association with at least one component with which it is naturally associated. Contaminant components of its natural environment are materials that would typically interfere with diagnostic or therapeutic uses for the polypeptide and may include enzymes, and other proteinaceous or non-proteinaceous solutes. An isolated polypeptide includes polypeptide in situ within recombinant cells, since at least one component of the p28^{TEV} polypeptide natural environment will not be present. Ordinarily, however, an isolated polypeptide will be prepared by at least one purification step.

An “isolated” nucleic acid molecule encoding a p28^{TEV} polypeptide is a nucleic acid molecule that is identified and separated from at least one contaminant nucleic acid molecule with which it is ordinarily associated in the natural source of the p28^{TEV}-encoding nucleic acid. Preferably, the isolated nucleic is free of association with all components with which it is naturally associated. An isolated p28^{TEV}-encoding nucleic acid molecule is other than in the form or setting in which it is found in nature. Isolated nucleic acid molecules therefore are distinguished from the p28^{TEV}-encoding nucleic acid molecule as it exists in natural cells or virus. In an embodiment, the nucleic acid molecule comprises a nucleic acid sequence of SEQ ID NO:2. In other embodiment, the nucleic acid molecule comprises a nucleic acid sequence selected from the group consisting of SEQ ID NO:2, SEQ ID NO:141, SEQ ID NO:142, SEQ ID NO:144, SEQ ID NO:145, SEQ ID NO:147, SEQ ID NO:148, SEQ ID NO:149, and SEQ ID NO:150. In another embodiment, the nucleic acid molecule encodes a p28^{TEV} polypeptide comprising the amino acid sequence of SEQ ID NO:151 or variants thereof. In other embodiments, the nucleic acid molecule encodes a p28^{TEV} polypeptide that comprises an amino acid sequence selected from the group consisting of SEQ ID NO:1, SEQ ID NO:143, SEQ ID NO:146, SEQ ID NO:151, SEQ ID NO:137, SEQ ID NO:138, SEQ ID NO:139, SEQ ID NO:140, SEQ ID NO:152, SEQ ID NO:153, SEQ ID NO:154, SEQ ID NO:155, and SEQ ID NO:156.

The disclosure also includes variants of nucleic acid molecules encoding p28^{TEV} polypeptides. In one embodiment, the disclosure includes polynucleotides encoding a polypeptide having at least about any number of sequence identity from 70% to 100% sequence identity to the reference polypeptide for p28^{TEV}, more preferably about 70% sequence identity, more preferably about 75% sequence identity, more preferably about 80% sequence identity, more preferably about 85% sequence identity, more preferably about 90% sequence identity, more preferably about 95% sequence identity, and even up to 100% sequence identity to a reference p28^{TEV} protein such as that having an amino acid sequence of SEQ ID NO:151. In other embodiments, a p28^{TEV} reference polypeptide comprises an amino acid sequence selected from the group consisting of SEQ ID NO:1, SEQ ID NO:143, SEQ ID NO:146, SEQ ID NO:151, SEQ ID NO:137, SEQ ID NO:138, SEQ ID NO:139, SEQ ID NO:140, SEQ ID NO:152, SEQ ID NO:153, SEQ ID NO:154, SEQ ID NO:155, and SEQ ID NO:156. In some embodiments, the polynucleotide variants encode a polypeptide that increases Tat transcriptional activity in the HIV-1-Luc assay.

The term “control sequences” refers to DNA sequences necessary for the expression of an operably linked coding sequence in a particular host organism. The control sequences that are suitable include, for example, a promoter, and optionally an enhancer sequence.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

Peptide and protein sequences defined herein are represented by one-letter symbols for amino acid residues as follows:

| | | | |
|---|---|---|---|
| A | alanine | L | leucine |
| R | arginine | K | lysine |
| N | asparagine | M | methionine |
| D | aspartic acid | F | phenylalanine |
| C | cysteine | P | proline |
| Q | glutamine | S | serine |
| E | glutamic acid | T | threonine |
| G | glycine | W | tryptophan |
| H | histidine | Y | tyrosine |
| I | isoleucine | V | valine |

“Percent (%) amino acid sequence identity” with respect to the p28^{TEV} polypeptide sequences identified herein is defined as the percentage of amino acid residues in a candidate sequence that are identical with the amino acid residues in a p28^{TEV} polypeptide reference sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, clustal V (DNASTAR) or Megalign (DNASTAR) software. Those skilled in the art can determine appropriate parameters for measuring alignment, including any algorithms needed to achieve maximal alignment over the full-length of the sequences being compared. Alignments of some HIV proteins from different clades can be found at the Los Alamos website (http://www-hiv-lanl-gov/content/index).

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction X/Y
where X is the number of amino acid residues scored as identical matches by the sequence alignment program in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Typically, the B amino acid sequence is that of SEQ ID NO:1.

“Percent (%) nucleic acid sequence identity” with respect to the p28^{TEV} polypeptide-encoding nucleic acid sequences identified herein is defined as the percentage of nucleotides in a candidate sequence that are identical with the nucleotides in a reference p28^{TEV} polypeptide-encoding nucleic acid sequence, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN, or Megalign (DNASTAR) software. Alignments of some HIV nucleic acids from different clades can be found at the Los Alamos website (http://www-hiv-lanl-gov/content/index). Those skilled in the art can determine appropriate parameters for measuring alignment, including any algorithms needed to achieve maximal alignment over the full-length of the sequences being compared.

For purposes herein, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:
100 times the fraction W/Z
where W is the number of nucleotides scored as identical matches by the sequence alignment program in that program's alignment of C and D, and where Z is the total number of nucleotides in D. It will he appreciated that where the length of nucleic acid sequence C is not equal to the length of nucleic acid sequence D, the % nucleic acid sequence identity of C to D will not equal the % nucleic acid sequence identity of D to C.

The term “antibody” is used in the broadest sense and specifically includes, for example, single anti-p28^{TEV} monoclonal antibodies, anti-p28TEv antibody compositions with polyepitopic specificity, human antibodies, humanized antibodies, single chain anti-p28^{TEV} antibodies, and fragments of anti-p28^{TEV} antibodies (see below). The term “monoclonal antibody” as used herein refers to an antibody obtained from a population of substantially homogeneous antibodies, i.e., the individual antibodies comprising the population are identical except for possible naturally-occurring mutations that may be present in minor amounts.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies; single-chain antibody molecules; and multispecific antibodies formed from antibody fragments.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The term “epitope tagged” when used herein refers to a chimeric polypeptide comprising a p28^{TEV} polypeptide linked to a “peptide tag”. The peptide tag has enough residues to provide an epitope against which an antibody can be made, yet is short enough such that it does not interfere with activity of the polypeptide to which it is fused. The peptide tag preferably also is fairly unique so that the antibody does not substantially cross-react with other epitopes. Suitable peptide tags generally have at least six amino acid residues and usually between about 8 and 50 amino acid residues (preferably, between about 10 and 20 amino acid residues).

As used herein “fusion protein” refers to an additional polypeptide that is linked to a p28^{TEV} polypeptide, preferably, at the N and/or C terminal end. The additional polypeptide is preferably a peptide tag that provides for ease of purification or identification. Additional peptides or polypeptides may also be fused to enhance immunogenicity, such as bovine serum albumin, or keyhole lymphocyte hemocyanin.

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. Isolated antibody includes the antibody iii situ within recombinant cells since at least one component of the antibody's natural environment will not be present. Ordinarily, however, isolated antibody will be prepared by at least one purification step.

The term “immunogenic effective amount” of a p28^{TEV} polypeptide or polynucleotide disclosed herein refers to an amount of a polypeptide that is capable of inducing an immune response in an animal. The immune response may be determined by measuring a T or B cell response. Typically, the induction of an immune response is determined by the detection of antibodies specific for a p28^{TEV} polypeptide.

As used herein, the term “immunogenic fragment thereof” refers to a fragment a p28^{TEV} polypeptide that is of a sufficient size to elicit an immune response in an animal. Typically, immunogenic fragments are at least 8 amino acids long and may include up to the full-length polypeptide. In some embodiments, an immunogenic fragment is about 10 amino acids, 15 amino acids, 30 amino acids, or 45 amino acids. The immunogenic fragment is capable of stimulating an antibody or selecting for an antibody that specifically binds to at least one p28^{TEV} polypeptide. In some embodiments, the immunogenic fragment includes overlapping polypeptides in the V1 region of a p28^{TEV} polypeptide. The immune response includes both a T and B cell response, but preferably is identified by the ability of the fragment to elicit antibodies.

The term “binds specifically” refers to an antibody that binds to a particular p28^{TEV} polypeptide. In some embodiments, the antibodies are specific for peptides of the V1 region. In some embodiments, the antibody specifically binds p28^{TEV} and does not bind to Tat or Rev. In other embodiments, the antibody that binds to p28^{TEV} may crossreact with Tat and/or Rev, but binds to p28^{TEV} with a higher affinity, preferably at least 100 fold higher affinity, more preferably 1000 fold higher affinity or greater than binding of the antibody to Tat and/or Rev in order to allow for differential detection of p28^{TEV} from tat or rev proteins.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCI, 0.075 M sodium citrate), 50 mM sodium phosphate (pH 6.8), 0.1% sodium pyrophosphate, 5× Denhardt's solution, sonicated salmon sperm DNA (50 g/ml), 0.1% SDS, and 10% dextran sulfate at 42° C., with washes at 42° C. in 0.2×SW (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5× Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, in etc. as necessary to accommodate factors such as probe length and the like.

As used herein “recombinant” refers to a nucleic acid molecule that has been isolated and/or altered by the hand of man. Typically a DNA sequence encoding a polypeptide is isolated and combined with other control sequences in a vector. The other control sequences may be those that are found in the naturally occurring gene or others. The vector provides for introduction into host cells, amplification of the nucleic acid sequence and expression of the nucleic acid sequence.

As used herein, the term “treating” refers to therapeutic treatment Those in need of treatment include those already infected with HIV, especially those having a reservoir of virus.

“siRNA” refers to a small interfering RNA that is a short-length double-stranded RNA and is not toxic in mammalian cells. siRNA induces gene-specific suppression through sequence specific degradation of homologous gene transcripts. P. Sharp, 1999, Genes & Devlop., 13:139-141; Berstein et al., 2000, RNA, 97:4985; U.S. 20030220287. There is no particular limitation in the length of siRNA. U.S. 20040002077. The siRNA may be 15 to 50 bp long, preferably 15 to 35 bp, and more preferably 21 to 30 bp. The double-stranded RNA portions of siRNAs may contain nonpairing nucleotides due to mismatchs, wherein the corresponding nucleotides are not complementary, and/or bulges, wherein the corresponding complementary nucleotide is lacking on one strand. See U.S. 20040002077. Double stranded RNA (dsRNA) may comprise nonpairing nucleotides to the extent the nonpairing nucleotides do not interfere with siRNA formation.

“Antisense RNA” refers to a single strand RNA comprising a sequence that is complementary to a target mRNA and not toxic in mammalian cells. There is no particular limitation in the length of antisense RNA. See U.S. 20030220287. The length of antisense RNA is dependent upon the number and composition of complementary bases and the accessibility of the target sequence. U.S. 20030220287.

A. Immunogenic Compositions

An embodiment of the present disclosure provides an immunogenic composition or pharmaceutical composition including at least one isolated and/or purified p28^{TEV} polypeptide, an isolated nucleic acid encoding at least one p28^{TEV} polypeptide, or both. In some embodiments, the immunogenic composition comprises an isolated naturally occurring p28^{TEV} polypeptide or isolated nucleic acid encoding a naturally occurring p28^{TEV} polypeptide, or both, from more than one clade of HIV. Preferably, an immunogenic composition comprises at least two and up to six different polynucleotides and/or polypeptides. A single nucleic acid may encode more than one p28^{TEV} polypeptide and/or a nucleic acid may encode a single p28^{TEV} polypeptide and/or combinations thereof. In some embodiments, compositions contain an amount of p28^{TEV} polypeptide and/or polynucleotide effective to elicit an immune response when administered to a host, for example, a mammal. The immune response can be humoral, cellular, or a both. Generally, the immune response inhibits the HIV viral levels in the immunized host compared to HIV viral levels in non-immunized hosts. The immunogenic composition optionally includes a pharmaceutically acceptable excipient or carrier.

An embodiment provides an immunogenic composition according to the present disclosure also including immunomodulators such as cytokines or chemokines. In some embodiments, a nucleic acid encodes the immunomodulator or adjuvant. Immunomodulators refers to substances that potentiate an immunogenic response including, but not limited to cytokines and chemokines. Examples of cytokines include but are not limited to IL-15, IL-12, or GM-CSF. Adjuvants such as lipids (fatty acids, phospholipids, Freund's incomplete adjuvant in particular), anionic copolymers, CpG units, etc. may be added to the composition.

In certain embodiments, the immunogenic composition comprises at least one isolated nucleic acid encoding a p28^{TEV} polypeptides or fragments thereof, recombinant vector or DNA. Recombinant vector refers to vectors that replicate in a host and express an antigen such as p28^{TEV}. One example of such a vector is a poxvirus. Poxviruses (including canarypox, vaccinia, and fowlpox) are suitable for recombinant vectors comprising an immunogenic composition disclosed herein. Poxviruses are capable of accommodating large amounts of foreign DNA and can infect mammalian cells, resulting in expression of a large amount of foreign protein. Fowlpox virus (FPV) is a member of the Poxviridae family (genus Avipoxvirus). Productive infection by fowlpox virus is restricted in vivo to avian species and in vitro to cells derived from avian species. However, inoculation of mammalian cells with avipox-based recombinants results in expression of foreign genes, and inoculation of mammals results in the induction of protective immunity.

Another embodiment provides an immunogenic composition based on modified vaccinia virus Ankara (MVA) expressing p28^{TEV} or an immunogenic fragment thereof. MVA is a highly attenuated strain of vaccinia virus that was developed toward the end of the campaign for the eradication of smallpox. Produced by hundreds of passages of vaccinia virus in chicken cells, MVA has lost about 10% of the vaccinia genome and with it the ability to replicate efficiently in primate cells. Despite its limited replication, MVA provides similar levels of recombinant gene expression to those of replication-competent vaccinia viruses in human cells.

Another embodiment provides an immunogenic composition based on non-replicating viral vectors made by deleting one or more genes from a virus capable of entering human cells. Nucleic acids encoding at least one p28^{TEV} polypeptide and optionally, one or more additional HIV antigens or cytokines replace the deleted viral gene resulting in a replication-incompetent viral vector. The antigens may be processed for presentation on the cell surface in association with MHC class 1. Antigens presented in this way to CD8 T cells can elicit an HIV-specific cytotoxic T-cell response.

Representative non-replicating viral vectors include, but are not limited to, gene-deleted adenovirus constructs (“adenovectors”) such as vectors derived from adenovirus type 5 (Ad5) which have been rendered replication-incompetent by deletion or inactivation of the E1 region (and in some cases other adenoviral genes as well). Adenovectors can contain deletions in multiple coding regions. The “missing” adenoviral gene products can be supplied by packaging cell lines specifically engineered to produce the vectors, but are not subsequently produced by the vectors themselves.

An embodiment provides an immuogenic composition comprising at least one naked DNA or a naked RNA encoding at least one polypeptide according to the disclosure. Naked DNA or RNA is DNA or RNA that does not have proteins or lipids associated with it.

A representative combination immunogenic composition would include, for example, gp120 in combination with a nucleic acid encoding p28^{TEV}.

In an embodiment, the immunogenic composition includes a plasmid that includes a gene encoding at least one p28^{TEV} polypeptide and optionally, at least one additional HIV antigen and/or immunomodulator such as IL-2, under the transcriptional control of a promoter region active in human cells. The coding region of p28^{TEV} is followed by transcription termination and polyadenylation sequences. To permit selection of plasmid-containing bacteria during the production process, the plasmid also contains an antibiotic resistance gene with a bacterial origin of replication. DNA is generally less costly to produce than peptide or protein, and is chemically stable under a variety of conditions. DNA is generally administered intramuscularly, using either a needle and syringe or a needle-free injector.

In another embodiment, at least one p28^{TEV} polypeptide according to the disclosure makes up the composition of a lipopeptide or of a lipoprotein. The association of a p28^{TEV} peptide with a fatty acid or phospholipid can potentiate an immunogenic response compared to the response caused by administering the p28^{TEV} peptide antigen alone. The p28^{TEV} compositions according to the disclosure may be coupled to microparticles or nanoparticles consisting of a polysaccharide core and/or covered in particular with a lipid bilayer. They may also be coupled to one or more liposomes or one or more niosomes (nonionic surfactant vesicles).

In some embodiments, an immunogenic composition can comprise at least one polynucleotide encoding a V1 peptide from the V1 region of p28^{TEV}. In some embodiments, a V1 peptide corresponds to amino acids 75-103 of p28^{TEV} of 89.6 (the sequence starting at the cysteine at position 75 and ending at the cysteine at position 103). (FIG. 33) In some embodiments, the peptide comprises sequence of:

20 NLNITKNTTNPTSSS, (SEQ ID NO:136)
21 TKNTTNPTSSSWGMM, (SEQ ID NO:137)
22 TNPTSSSWGMMEKGE, (SEQ ID NO:138)
23 SSSWGMMEKGEIKNC, (SEQ ID NO:139)
or
24 GMMEKGEIKNCSFYI. (SEQ ID NO:140)

In other embodiments, the V1 polypeptide comprises an amino acid sequence selected from the group consisting of:

SSSWGMMEKGE, (SEQ ID NO:152)
SSSRGMVGGGE, (SEQ ID NO:153)
SSNWKEMDRGE, (SEQ ID NO:154)
and
SSSGRMIMEKG. (SEQ ID NO:155)

In other embodiments, a V1 consensus sequence comprises a formula of contiguous amino acids comprising SSSX₄X₅MX₇X₈X₉GE (SEQ ID NO:156); wherein X4 is W, R, or G; X5 is G, K, or R; X7 is M, V or I; X8 is E,G,D, or M; and X9 is K,G,R, or E.

In other embodiments, an isolated and purified V1 polypeptide comprises at least one V1 consensus sequence of a clade as shown in FIG. 35. In some embodiments, an isolated V1 consensus sequence comprises:

for clade A:

(SEQ ID NO:164)

CSNX₃X₄NNTX₈X₉X₁₀NTNX₁₄TDGMREEKNC
for clade B:

(SEQ ID NO:165)

CTDLNNTNX₉X₁₀TSSSGGTMEKGEIKNC
for clade C:

(SEQ ID NO:166)

CTNVNINX₇TX₉X₁₀GX₁₂NTYNSMX₁₉X₁₀EIKNC
for clade D:

(SEQ ID NO:167)

CTDASRNX₈TX₁₀X₁₁NTNGPX₁₇MEKGEMKNC
for clade G:

(SEQ ID NO:168)

CTNVNNX₇X₈X₉X₁₀X₁₁TX₁₃NNX₁₆TVTX₂₀EEEKNC
for clade O:

(SEQ ID NO:169)

CTNX₄X₅GTTX₉X₁₀X₁₁X₁₂X₁₃X₁₄X₁₅X₁₆X₁₇X₁₈ENLMKQC;

wherein an X amino acid is any of the 20 naturally occurring amino acids.

Another embodiment provides an immunogenic composition comprising p28^{TEV} polypeptide or fragment thereof, or a p28^{TEV} polynucleotide, at least one additional HIV antigen, a polynucleotide encoding an additional HIV antigen, at least one immunomodulator, or combinations thereof. Suitable additional HIV antigens include, but are not limited to Vif, Tat, Gag, Env, Rev, gp120, gp41, p24, p7, p17, combinations thereof as well as polypeptides encoded by gag, nef, pol, env, vpr, vpu, a combination thereof, or immunogenic fragments thereof.

In an embodiment, a subject is initially primed with a polynucleotide encoding p28^{TEV} polypeptide in one vector. In some embodiments, the host is subsequently dosed with a polynucleotide encoding a p28^{TEV} polypeptide in a second vector which is different from the first vector. This approach has also been termed “heterologous boosting,” to distinguish it from the traditional method (homologous boosting) in which two or more doses of the same immunogenic composition are given successively. Heterologous boosting helps to minimize immunogenic response to the vector itself. The dose used to boost the immune response can include one more cytokines, chemokines, immunomodulators, or HIV antigens not present in the priming dose of the immunogenic composition. In a further embodiment, the subject is subsequently boosted with one or more p28^{TEV} polypeptides or fragment thereof, and optionally an immunomodulator or adjuvant.

In some embodiments, naturally occurring p28^{TEV} polynucleotides may be isolated by cloning out virus from infected individuals and selectively amplifying polynucleotide region encoding p28^{TEV} and expressing these polynucleotides to isolate naturally occurring p28^{TEV} polypeptides present in infected individuals at different stages of infection. Primers for amplifying p28^{TEV} sequences can be designed from the sequences provided in FIGS. 26-33. Such polynucleotides or polypeptides may be useful in the immunogenic compositions described herein.

B. p28^{TEV} Polypeptides

HIV-1 encodes alternatively spliced mRNAs for several proteins that regulate viral transcription and replication. Among them, two mRNAs that encode two proteins, p18 and p28^{TEV}, have been described. Benko et al., 1990, J Virol., 64:2505-2518. HIV-1 p28^{TEV} is encoded by alternatively spliced mRNA that joins together the first exon of Tat to a region with the Env (exon 6D) and to the second exon of Rev. This protein is expressed early in infection and in fact, is the first protein expressed during infection. Benko et al., 1990, J. Virol., 64:2505-2518.

The p28^{TEV} is a phosphoprotein and is immunopreciptated with both anti-tat and anti-rev monoclonal antibodies. Since humans develop antibodies to Tat and Rev, p28^{TEV} likely is recognized by sera of HIV-1-infected humans. p28^{TEV} has both tat and rev functions, although the rev function is not as strong as that of rev protein itself. The amino terminal of p28^{TEV} has the first exon of tat and functional studies show that the first 58 amino acids of exon 1 are sufficient for full tat activity. p28^{TEV} appears to have comparable activity to that of tat alone. The carboxy terminus of p28^{TEV} has the second exon of rev including the domain that may act as a nuclear localization signal. The exon of the Env gene of p28^{TEV} includes the V1 Env region of the virus, which is highly variable. Therefore, naturally occurring p28^{TEV} polypeptides have amino acid sequence variability at least in the portion of the p28^{TEV} polypeptide encoded by the V1 Env region. p28^{TEV} is also found extracellularly. In an embodiment, an isolated and purified p28^{TEV} polypeptide comprises an amino acid sequence of SEQ ID NO:151. In other embodiments, an isolated and purified p28^{TEV} polypeptide comprises an amino acid sequence selected from the group consisting of SEQ ID NO:1, SEQ ID NO:143, SEQ ID NO:146, and SEQ ID NO:151.

A comparison of HIV-1 proviruses shows that exon 6D is conserved in some of the IIIB family of HIV-1 molecular clones that belong to clade B of HIV. Benko et al., 1990, J. Virol., 64:2505-2518. Other proviral clones lack one or both of the exon 6D splice cites, but may contain alternative splice sites in the vicinity of the exon 6D sequence (Benko et al., 1990, J. Virol., 64:2505-2518) such that proviral clones lacking exact matches for the exon 6D splice acceptor site and splice donor site may generate naturally occurring p28^{TEV} variant polypeptides through recognition of other alternative splice sites in the Env gene.

V1 regions of p28^{TEV} polypeptides of other HIV isolates corresponding to amino acids 75 to 103 of p28^{TEV} of isolate 89.6 can be identified by finding the sequences and aligning them using the tools, for example, provided at GenBank or the Los Alamos database.

In other embodiments, an isolated and purified V1 polypeptide comprises at least one V1 consensus sequence of a clade as shown in FIG. 35. In some embodiments, an isolated V1 consensus sequence comprises

for clade A:

(SEQ ID NO:164)

CSNX₃X₄NNTX₈X₉X₁₀NTNX₁₄TDGMREEKNC
for clade B:

(SEQ ID NO:165)

CTDLNNTNX₉X₁₀TSSSGGTMEKGEIKNC
for clade C:

(SEQ ID NO:166)

CTNVNINX₇TX₉X₁₀GX₁₂NTYNSMX₁₉X₂₀EIKNC
for clade D:

(SEQ ID NO:167)

CTDASRNX₈TX₁₀X₁₁NTNGPX₁₇MEKGEMKNC
for clade G:

(SEQ ID NO:168)

CTNVNNX₇X₈X₉X₁₀X₁₁TX₁₃NNX₁₆TVTX₂₀EEEKNC
for clade O:

(SEQ ID NO:169)

CTNX₄X₅GTTX₉X₁₀X₁₁X₁₂X₁₃X₁₄X₁₅X₁₆X₁₇X₁₈ENLMKQC;

wherein an X amino acid is any of the 20 naturally occurring amino acids.

Naturally occurring p28^{TEV} polypeptides may be identified by antibodies that specifically bind Tat, Rev, and/or the V1 Env region, or by at least 70% sequence identity to an amino acid sequence comprising SEQ ID NO:151 (FIG. 33). Preferably, naturally occurring p28^{TEV} polypeptides have a biological activity of a p28^{TEV} polypeptide reference sequence and is a polypeptide that is expressed early during infection. In a preferred embodiment, the biological activity is Tat transcriptional activity in a HIV-Luciferase assay as described herein.

C. p28^{TEV} Polypeptide Variants

The disclosure also encompasses p28^{TEV} polypeptide variants and immogenic compositions containing them. p28^{TEV} polypeptide variants can be prepared by introducing appropriate nucleotide changes into the p28^{TEV} polypeptide encoding DNA, and/or by synthesis of the desired p28^{TEV} polypeptide variant or isolated from naturally occurring sources. p28^{TEV} polypeptides can be isolated and purified using methods such as affinity purification. p28^{TEV} polypeptide variants may be useful as antagonists or to prepare antagonists.

A p28^{TEV} variant has at least about any one of 70% to 100% sequence identity to a full-length mature p28^{TEV} polypeptide reference sequence. A p28^{TEV} variant has at least about 70% sequence identity, more preferably at least about 75% sequence identity, more preferably at least about 80% sequence identity, more preferably at least about 85% sequence identity, more preferably at least about 90% sequence identity, more preferably at least about 95% sequence identity and even 100% sequence identity to a full-length mature p28^{TEV} polypeptide reference sequence, such as a polypeptide having the sequence of SEQ ID NO:151. Alternatively, a p28^{TEV} variant can have about 70 to 100% sequence identity to a reference peptide selected from the group consisting of SEQ ID NO:136, SEQ ID NO:137, SEQ ID NO:138, SEQ ID NO:139, SEQ ID NO:140 ,SEQ ID NO:152, SEQ ID NO:153, SEQ ID NO:154, SEQ ID NO:155, and SEQ ID NO: 156.

Preferably, the polypeptide variants increase Tat activity in the HIV-1-Luc assay. In some embodiments, the transcriptional activity is increased at least 2 fold, more preferably 2 to 5 fold compared to control. In one embodiment, the p28^{TEV} polypeptide is an isolated and purified polypeptide comprising an amino acid sequence of SEQ ID NO:151 (FIG. 33).

Variants include naturally occurring variants having the sequence of p28^{TEV} polypeptide isolated from nature from different HIV strains. Variations in the naturally occurring full-length p28^{TEV} polypeptides described herein, can also be made, for example, using any of the techniques and guidelines for conservative and non-conservative mutations set forth, for instance, in U.S. Pat. No. 5,364,934. Variations may be a substitution, deletion or insertion of one or more codons encoding the p28^{TEV} polypeptide that results in a change in the amino acid sequence of the p28^{TEV} polypeptide as compared with a naturally occurring p28^{TEV} polypeptide.

Guidance in determining which amino acid residue may be inserted, substituted or deleted without adversely affecting the desired activity may be found by comparing the sequence of the p28^{TEV} polypeptide with that of homologous known protein molecules and minimizing the number of amino acid sequence changes made in regions of high homology. Functional domains can also be identified in those p28^{TEV} polypeptides that have homology to known polypeptides.

Functional domains of p28^{TEV} are known. As described previously, p28^{TEV} includes a portion of the Tat protein, the V1 envelope and the Rev protein. The Tat activity of p28^{TEV} is similar to that of Tat, while the activity of the rev portion of p28^{TEV} is decreased compared to that of the rev protein. The amino terminal of p28^{TEV} has the first exon of Tat and functional studies show that the first 58 amino acids of exon 1 are sufficient for full tat activity. p28^{TEV} appears to have comparable activity to that of tat alone. The carboxy terminus of p28^{TEV} has the second exon of rev including the domain that may act as a nuclear localization signal. The exon of the Env gene of p28^{TEV} includes the V1 Env region of the virus, which is highly variable. Therefore, naturally occurring p28^{TEV} polypeptides may have amino acid sequence variability at least in the portion of the p28^{TEV} polypeptide encoded by the V1 Env region. See FIG. 35 to identify positions of V1 region that can be substituted without affecting function.

The sequences of these functional domains can be compared and aligned to other known sequences for Tat, V1 and Rev from other HIV-1 strains that may be provided at the Los Alamos website or GenBank, and locations of amino acid positions for substitutions can be identified as those positions that show a high degree of variability in amino acids, i.e., at least 3 different amino acids are found at that position when different sequences are aligned and compared or have a lower percentage of sequence identity i.e., less than 90% sequence identity. When sequences are aligned the positions that show variability can either have conservative amino acid substitutions or non-conservative amino acid substitutions. If the position has conservative amino acid substitutions that would indicate that the amino acid substituted at that position should be of the same type as those observed to be at that position in naturally occurring proteins. For examples of such substitutions, see Table 1.

For example, based on the known variability in the V1 region of the env, one or more amino acid substitutions can be made in this portion of p28^{TEV} without affecting function.

Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature naturally occurring sequence. Preferably, variants have the biological activity of the source molecule, such as increased Tat activity in the Luc assay.

In particular embodiments, conservative substitutions of interest are shown in Table 1 under the heading of preferred substitutions.

TABLE 1

| Original | | Preferred |
|---|---|---|
| Residue | Exemplary Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; norleucine | |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; type | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; norleucine | leu |

The variations can be made using methods known in the art such as oligonucleotide-mediated (site-directed) mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] or other known techniques can be performed on the cloned DNA to produce the p28^{TEV} polypeptide variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant [Cunningham and Wells, Science, 244: 1081-1085 (1989)]. Alanine is also typically preferred because it is the most common amino acid. Further, it is frequently found in both buried and exposed positions [Creighton, The Proteins, (W.1-1. Freeman & Co., N.Y.); Chothia, J. Mol. Biol., 150:1 (1976)]. If alanine substitution does not yield adequate amounts of variant, an isoteric amino acid can be used.

p28^{TEV} polypeptide variants also include naturally occurring polypeptide variants. A reference p28^{TEV} polypeptide is obtained from a single source of HIV-1, such as HXB2, and the sequence of this reference or source polypeptide may differ from the sequence of the same type of p28^{TEV} polypeptide obtained from either a strain from the same clade or from other strains from another clade. p28^{TEV} polypeptide variants may be identified using antibodies that specifically bind, for example, Tat, Rev, and/or the V1 Env region, or by having with sequence identity to an amino acid sequence of SEQ ID NO:1. For example, p28^{TEV} can be identified by binding to an anti-V1 and an anti-tat or anti-rev antibody.

p28^{TEV} polypeptide fragments are provided herein. Such fragments may be truncated at the N-terminus or C-terminus, or may lack internal residues, for example, when compared with a full-length protein. Certain fragments lack amino acid residues that are not essential for a desired biological activity of the p28^{TEV} polypeptide.

In some embodiments, an immunogenic composition can comprise at least one isolated and/or purified V1 peptide from the V1 region of p28^{TEV},preferably corresponding to amino acids 75-103 of p28^{TEV} of 89.6. In some embodiments, the peptide comprises a sequence of:

20 NLNITKNTTNPTSSS, (SEQ ID NO:136)
21 TKNTTNPTSSSWGMM, (SEQ ID NO:137)
22 TNPTSSSWGMMEKGE, (SEQ ID NO:138)
23 SSSWGMMEKGEIKNC, (SEQ ID NO:139)
or
24 GMMEKGEIKNCSFYI. (SEQ ID NO:140)

In other embodiments, the V1 polypeptide comprises an amino acid sequence selected from the group consisting of:

SSSWGMMEKGE, (SEQ ID NO:152)
SSSRGMVGGGE, (SEQ ID NO:153)
SSNWKEMDRGE, (SEQ ID NO:154)
and
SSSGRMIMEKG. (SEQ ID NO:155)

In other embodiments, a V1 consensus sequence comprises a formula of contiguous amino acids comprising SSSX₄X₅MX₇X₈X₉GE (SEQ ID NO:156); wherein X4 is W, R, or G; X5 is G, K, or R; X7 is M, V or I; X8 is E,G,D, or M; and X9 is K,G,R, or E.

In other embodiments, an isolated and purified V1 polypeptide comprises at least one V1 consensus sequence of a clade as shown in FIG. 35. In some embodiments, an isolated V1 consensus sequence comprises

for clade A:

(SEQ ID NO:164)

CSNX₃X₄NNTX₈X₉X₁₀NTNX₁₄TDGMREEKNC
for clade B:

(SEQ ID NO:165)

CTDLNNTNX₉X₁₀TSSSGGTMEKGEIKNC
for clade C:

(SEQ ID NO:166)

CTNVNINX₇TX₉X₁₀GX₁₂NTYNSMX₁₉X₁₀EIKNC
for clade D:

(SEQ ID NO:167)

CTDASRNX₈TX₁₀X₁₁NTNGPX₁₇MEKGEMKNC
for clade G:

(SEQ ID NO:168)

CTNVNNX₇X₈X₉X₁₀X₁₁TX₁₃NNX₁₆TVTX₂₀EEEKNC
for clade O:

(SEQ ID NO:169)

CTNX₄X₅GTTX₉X₁₀X₁₁X₁₂X₁₃X₁₄X₁₅X₁₆X₁₇X₁₈ENLMKQC;
wherein an X amino acid is any of the 20 naturally
occurring amino acids.

In some embodiments, one or more of the V1 peptides may be combined with a heterologous molecule such as a short peptide that could provide for maintenance of a loop structure. For example, providing a short peptide of 1 to 5 amino acids on both ends of the peptide including a cysteine would allow formation of a disulfide bond which would provide a loop of each of the V1 peptides. Alternatively, a portion of the V1 region of p28^{TEV} could be retained in order to provide for the loop structure of the V1 peptides, especially including the cysteines at positions 75 and 103 of 89.6 tev.

In some embodiments, the V1 peptides may be naturally occurring variants corresponding to the same region of the peptides 20-24 shown above. In other embodiments, variants of the peptides 20-24 can be derived using standard methods. Variants can have any number % sequence identity of about 70 to 100% sequence identity to a reference sequence such as that of SEQ ID NOS:136-140 or SEQ ID NOS: 152-156 . In addition, an immunogenic composition comprises one or more peptides corresponding to the peptide of SEQ ID NO:137, SEQ ID NO:138, SEQ ID NO:139, SEQ ID NO:140, SEQ ID NO:152, SEQ ID NO:153, SEQ ID NO:154, SEQ ID NO:155, or SEQ ID NO:156 from one or more HIV clades. In another embodiment, an immunogenic composition may comprise one or more consensus sequences for the V1 portion of p28^{TEV} or corresponding to the V1 peptides described herein.

The disclosure also contemplates immunogenic fragments of p28^{TEV} polypeptides. Immunogenic fragments are at least 8 amino acids in length, more preferably 8-50 amino acids, more preferably at least 10 amino acids, and more preferably at least 20 amino acids up to a full-length polypeptide. Immunogenic fragments can be prepared synthetically, recombinantly, or by enzymatic digestion of p28^{TEV} polypeptide. Immunogenic fragments can be predicted by analyzing the primary amino acid sequence of a p28^{TEV} polypeptide using commercially available services such as Epipredict or Epitope informatics or publicly available programs such as are available. The fragments can be used to select or generate an antibody that specifically binds to a p28^{TEV} polypeptide.

p28^{TEV} polypeptide fragments may be prepared by any of a number of conventional techniques. Desired peptide fragments may be chemically synthesized. An alternative approach involves generating p28^{TEV} polypeptide fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired polypeptide fragment, by polymerase chain reaction (PCR). Oligonucleotides that define the desired termini of the DNA fragment are employed at the 5′ and 3′ primers in the PCR. Preferably, p28^{TEV} polypeptide fragments share at least one biological and/or immunological activity with p28^{TEV} polypeptide comprising an amino acid sequence of SEQ ID NO:151.

Fusion proteins of the polypeptides or peptides described herein can also be prepared. The fusion protein can be attached to a heterologous moiety to provide a detectable label, increase immunogenicity or increase half life. Detectable labels include radionuclides, dyes, biotin, flag tag and the like. Polypeptides that increase immunogenicity include toxoids, glutathione reductase, albumin, and the like. Moieties that increase half life include polyethylene glycol.

D. Nucleic Acids

A second aspect of the disclosure relates to polynucleotides encoding p28^{TEV} polypeptides, recombinant vectors, and host cells containing the recombinant vectors, as well as methods of making such vectors and host cells by recombinant methods. The polynucleotides encoding p28^{TEV} or p28^{TEV} variants are useful as immunogenic compositions, to produce p28^{TEV} polypeptides or to prepare antagonists of p28^{TEV} expression.

The p28^{TEV} polynucleotides of the disclosure may be synthesized or prepared by techniques well known in the art. See, for example, Creighton, Proteins: Structures and Molecular Principles, W. H. Freeman & Co., New York, N.Y. (1983). Nucleotide sequences encoding the p28^{TEV} polypeptides of the disclosure may be synthesized, and/or cloned, and expressed according to techniques well known to those of ordinary skill in the art. See, for example, Sambrook, et al., Molecular Cloning, A Laboratory Manual, Vols. 1-3, Cold Spring Harbor Press, Cold Spring Harbor, N.Y. (1989).

The polynucleotides may be produced by standard recombinant methods known in the art, such as polymerase chain reaction (Sambrook, et al., 1989, Molecular Cloning, A Laboratory Manual, Vols. 1-3, Cold Spring Harbor Press, Cold Spring Harbor, N.Y.), or the DNA can be synthesized and optimized for expression in bacteria or eukaryotic cells. Primers can be prepared using the polynucleotide sequences provided, for example, in FIGS. 26-33 or that are available in publicly available databases. The polynucleotide constructs may be assembled from polymerase chain reaction cassettes sequentially cloned into a vector containing a selectable marker for propagation in a host. Such markers include dihydrofolate reductase or neomycin resistance for eukaryotic cell culture and tetracycline or ampicillin resistance genes for culturing in E. coli and other bacteria.

Representative examples of appropriate hosts include, but are not limited to, bacterial cells such as E. coli, Streptomyces and Salmonella typherium, fungal cells such as yeast; insect cells such as Drosophilia S2 and Spodoptera Sf9, animal cells such as CHO, COS, and Bowes melanoma cells, and plant cells. Appropriate culture medium and conditions for the above-described host cells are known in the art.

The polynucleotide should be operably linked to an appropriate promoter, such as CMV. Other suitable promoters are known in the art. The expression constructs may further contain sites for transcription initiation, transcription termination, and a ribosome binding site for translation. The coding portion of the mature polypeptide expressed by the constructs preferably includes a translation initiating codon at the beginning and a termination codon (UAA, UGA, or UAG) appropriately positioned at the end of the polypeptide to be translated.

Introduction of the recombinant vector into the host cell can be effected by calcium phosphate transfection, DEAE-dextran mediated transfection, cationic lipid-mediated transfection, electroporation, transduction, infection, or other methods. Such methods are described in standard laboratory manuals such as Sambrook, et al., 1989, Molecular Cloning, A Laboratory Manual, Vols. 1-3, Cold Spring Harbor Press, Cold Spring Harbor, N.Y. or Davis et al., 1986, Basic Methods in Molecular Biology. Commercial transfection reagents, such as Lipofectamine (Invitrogen, Carlsbad, Calif.), Effectene (Qiagen, Valencia, Calif.) and FuGENE 6™ (Roche Diagnostics, Indianapolis, Ind.), are also available.

The polypeptide, or fragment thereof, may be expressed in a modified form, such as a fusion protein, and may include secretion signals and/or additional heterologous functional regions. For example, a region of additional amino acids may be added to the N-terminus or C-terminus of the polypeptide to facilitate detection or purification, improve immunogenicity, improve half-life, or improve persistence in the host cell during, for example, purification or subsequent handling and storage. Examples of additional amino acids include peptide tags that may be added to the polypeptide to facilitate detection and/or purification. Such peptide tags include, but are not limited to, His, HA, Avi, biotin, c-Myc, VSV-G, HSV, V5, or FLAG™. Examples of a polypeptide that can enhance immunogenicity include bovine serum albumin, and/or keyhole lymphocyte hemocyanin. Examples of molecules that improve half life include polyethylene glycol.

The polypeptide can be recovered and purified from recombinant cell cultures by methods known in the art, including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. In an embodiment, high performance liquid chromatography (HPLC) is employed for purification.

The disclosure also includes variants of a polynucleotide encoding a p28^{TEV} protein having a sequence of SEQ ID NO:151. In still another embodiment, the polypeptide is selected from the group consisting a polypeptide comprising the amino acid sequence of SEQ ID NO:1 SEQ ID NO:143, SEQ ID NO:146, and SEQ ID NO:151. These variant polynucleotide sequences may encode naturally occurring variants of p28^{TEV} obtained from different HIV-1 strains or clades. The variants may also be made by substitution of nucleotides using standard methods.

Naturally occurring polynucleotides encoding variants of p28^{TEV} polypeptides can be isolated from cloning out viral isolates from infected individuals at various times post infection. Such polynucleotides can be obtained using primers for amplifying polynucleotide encoding p28^{TEV}. Such polynucleotides or polypeptides may be utilized in the immunogenic compositions described herein.

The disclosure also includes variants of nucleic acid molecules encoding p28^{TEV} polypeptides. In some embodiments, the disclosure includes polynucleotides having at least about 70% sequence identity, more preferably about 75% sequence identity, more preferably about 80% sequence identity, more preferably about 85% sequence identity, more preferably about 90% sequence identity, more preferably about 95% sequence identity, and even up to 100% sequence identity to a polynucleotide sequence encoding a p28^{TEV} protein having an amino acid sequence of SEQ ID NO:151. In some embodiments, the polynucleotide variants encode a polypeptide that has a biological activity of p28^{TEV} of increased Tat activity in a Luc assay.

In some embodiments, the disclosure includes polynucleotides encoding a polypeptide having at least about 70% sequence identity, more preferably about 75% sequence identity, more preferably about 80% sequence identity, more preferably about 85% sequence identity, more preferably about 90% sequence identity, more preferably about 95% sequence identity, and even up to 100% sequence identity to a polypeptide sequence having an amino acid sequence of SEQ ID NO:151. In still another embodiment, the reference polypeptide is selected from the group consisting a polypeptide comprising the amino acid sequence of SEQ ID NO:1, SEQ ID NO:143, SEQ ID NO:146, SEQ ID NO:151, SEQ ID NO:137, SEQ ID NO:138, SEQ ID NO:139, SEQ ID NO:140, SEQ ID NO:152, SEQ ID NO:153, SEQ ID NO:154, SEQ ID NO:155, and SEQ ID NO:156. In some embodiments, the polynucleotide variants encode a polypeptide that has a biological activity of p28^{TEV} of increased transcriptional activity in a Luc assay.

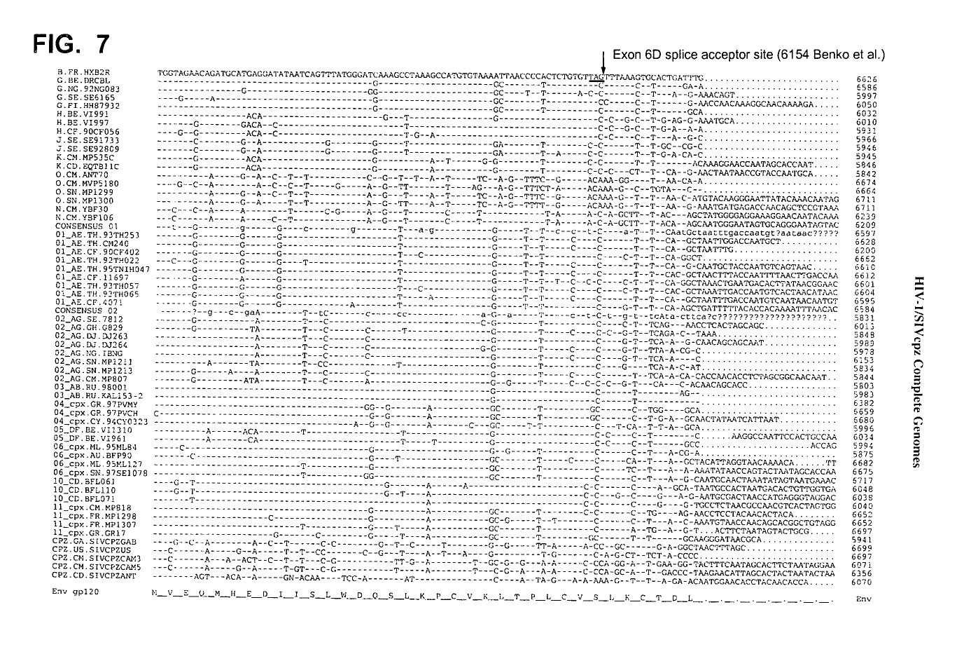

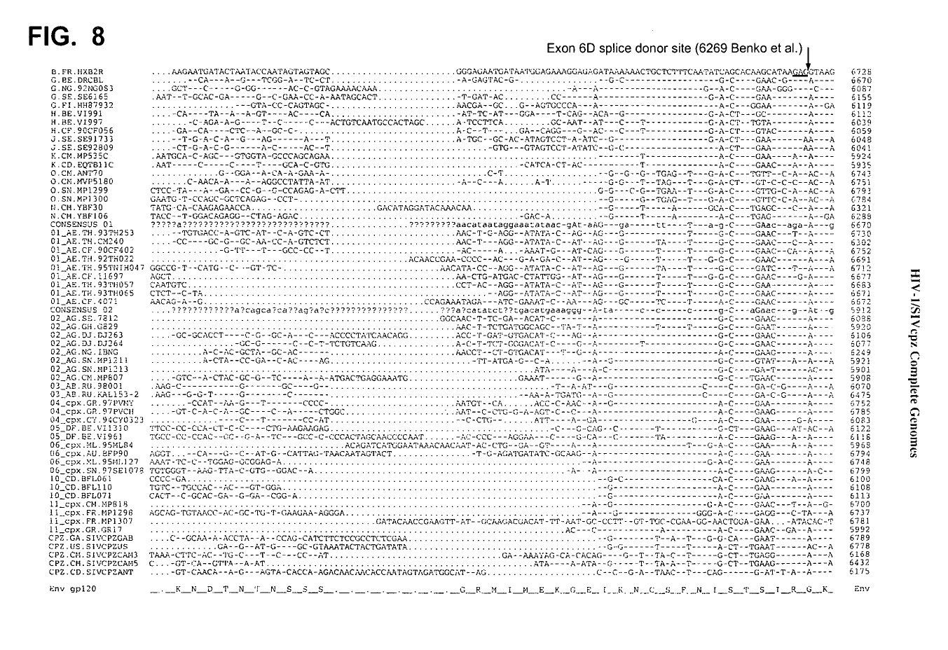

Polynucleotide variants include variants made at the splice acceptor site in the Env region at position 6154 and/or the splice donor site in the Env region at position 6269 as shown in FIGS. 7 and 8 and/or other cryptic acceptor splice sites or donor splice sites in p28^{TEV} encoding DNA or mRNA. Targeting this splice acceptor site splice and/or donor splice site by reprogramming alternative pre-mRNA splicing may prevent alternative splicing of p28^{TEV} and allow the virus to be in switched on mode (e.g. preferentially express Tat and replicate), making it more accessible to the immune system. Sazani et al., 2002, Nuc. Acids. Res., 29:3965-3974; Sanzani et al., 2002, Nature Biotechnol., 20:1228-1233; Villemaire et al., 2003, J. Biol. Chem., 278:50031-50039.

Vectors that are useful for expression of the polynucleotides of the disclosure include plasmid vectors as well as viral vectors. Several viral vectors are known to be useful in transducing DNA and/or RNA, such as lentivirus-based vectors.

The nucleic acids disclosed herein are useful in immunogenic compositions as described herein.

E. Nucleic Acid Antagonists of p28^{TEV} expression

Novel therapeutics can be identified that can inhibit the expression and/or activity of these p28^{TEV} polypeptides. Inhibition of the activity and/or expression of the p28^{TEV} polypeptide may inhibit HIV viral levels of some isolates.

Agents that can act therapeutically to inhibit the effect of p28^{TEV} on HIV-1 viral levles include p28^{TEV} variants, antagonist antibodies, antisense RNA specific for p28^{TEV}, small interfering RNA (siRNA), microRNA or methods to modulate pre-mRNA splice site choice. In some embodiments, antagonists are targeted to inhibit p28^{TEV} expression. The expression level of p28^{TEV} can be measured by transcriptional activity using HIV luciferase or by reverse transcriptase activity.

Antisense RNA can be prepared based on the nucleic acid sequence encoding p28^{TEV}, for example, SEQ ID NO:2. The p28^{TEV} mRNA can be specifically eliminated by introduction or expression of antisense sequences targeted to the unique p28^{TEV} sequences. Preferably, the antisense RNA is complementary to and/or binds under stringent conditions to a portion of the nucleic acid sequence including the splice acceptor site or splice donor site unique to p28^{TEV}.

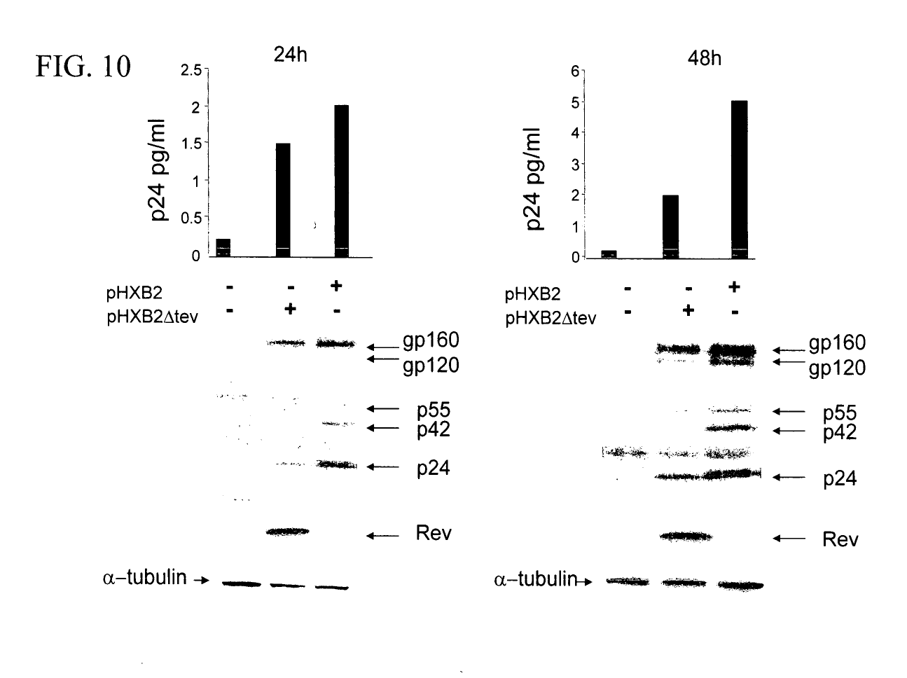

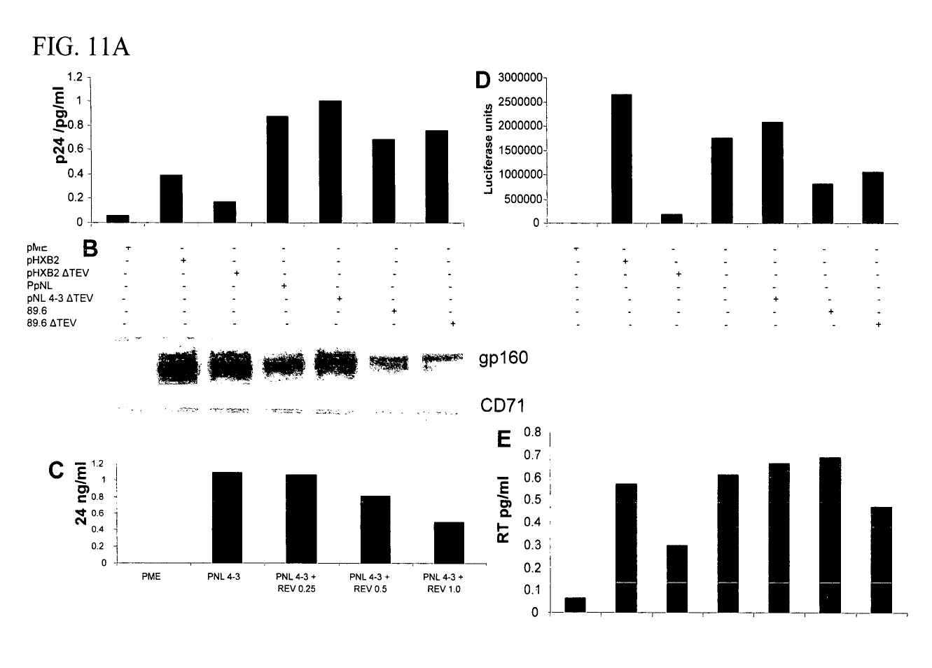

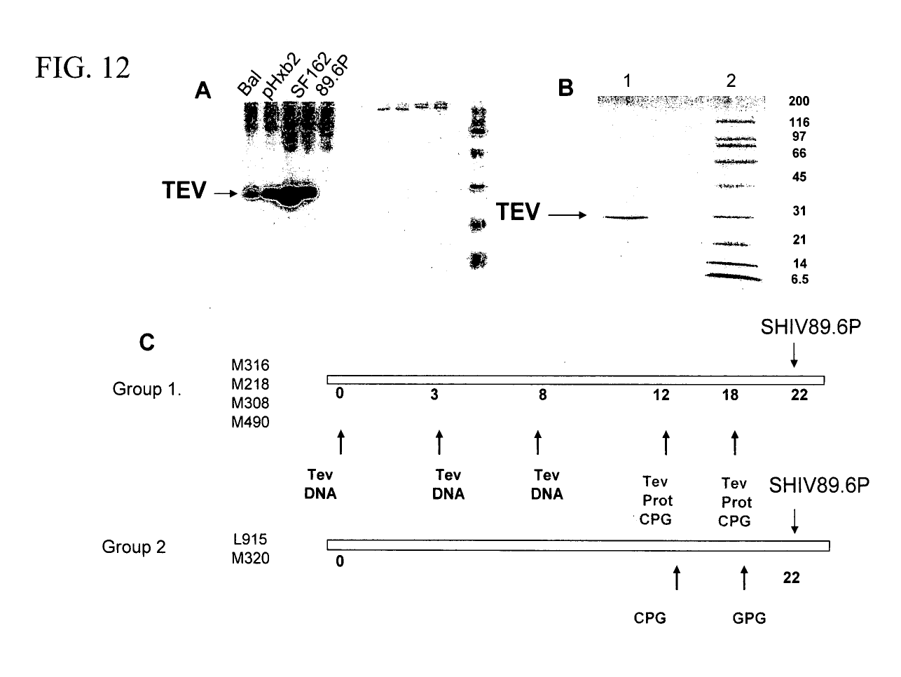

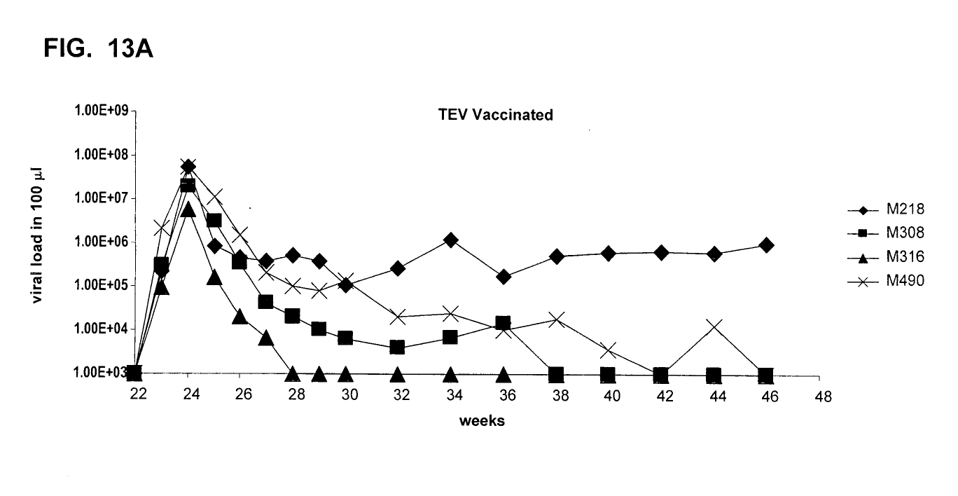

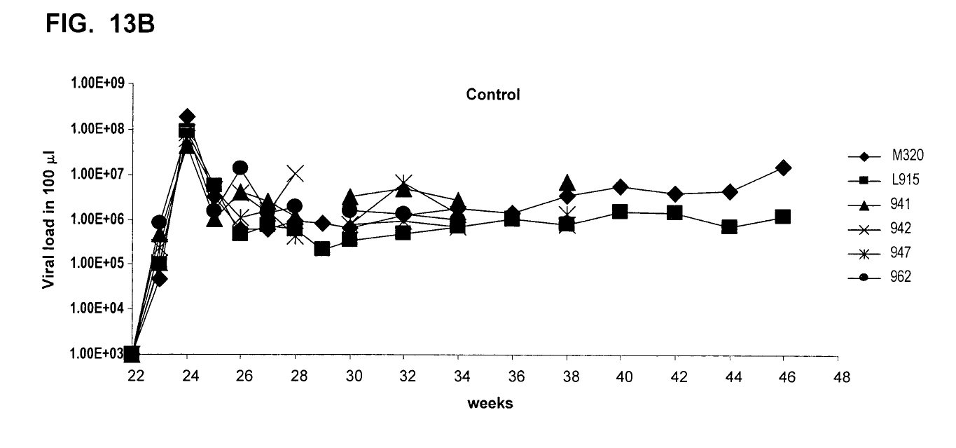

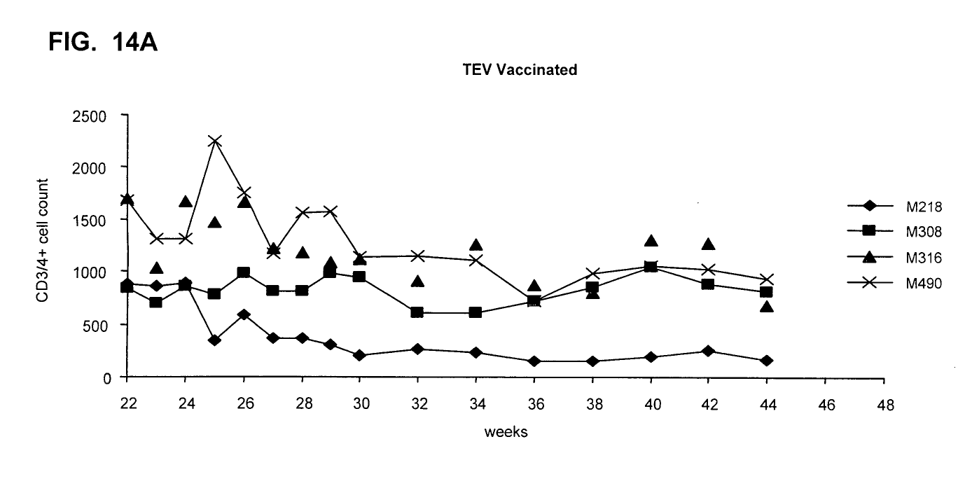

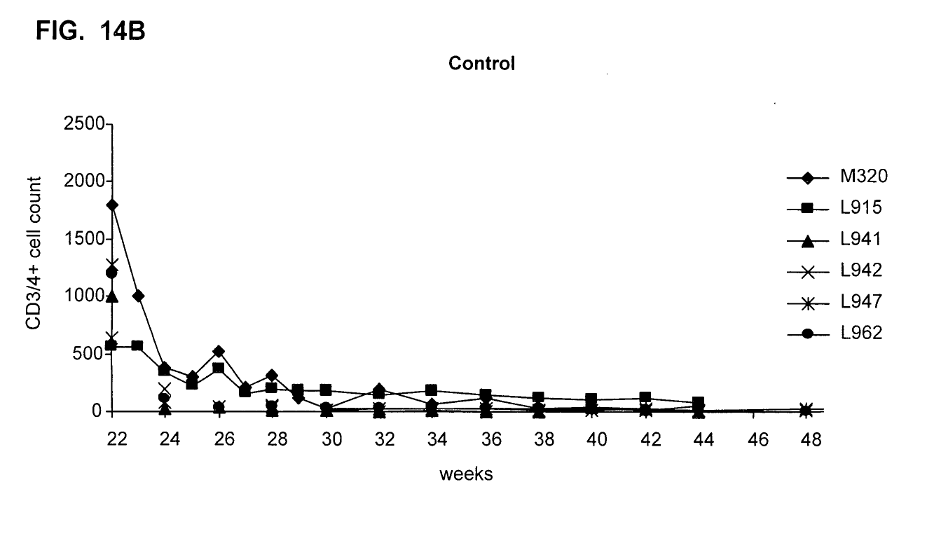

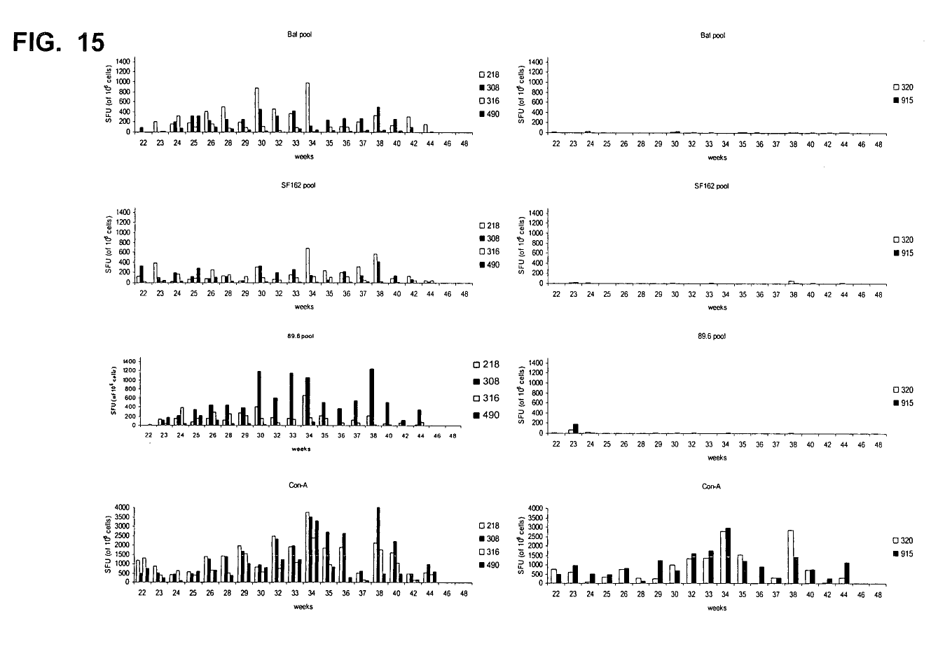

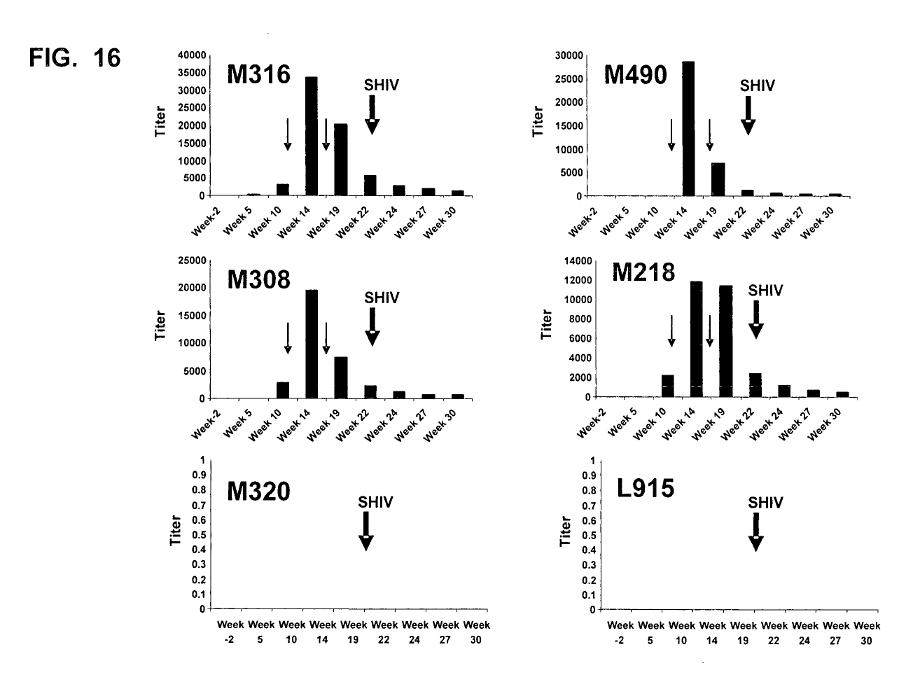

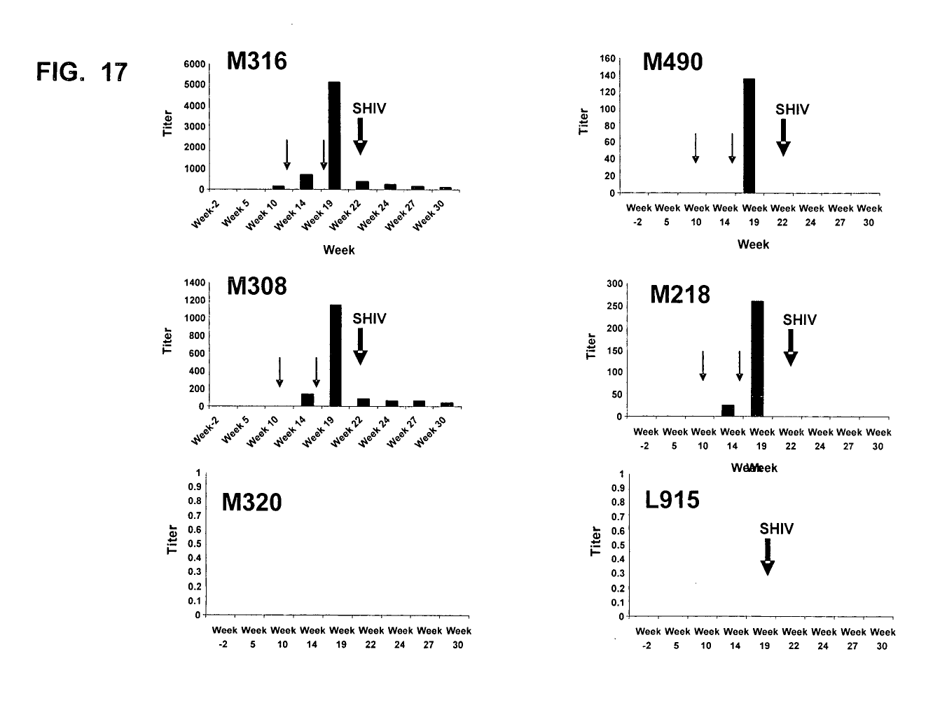

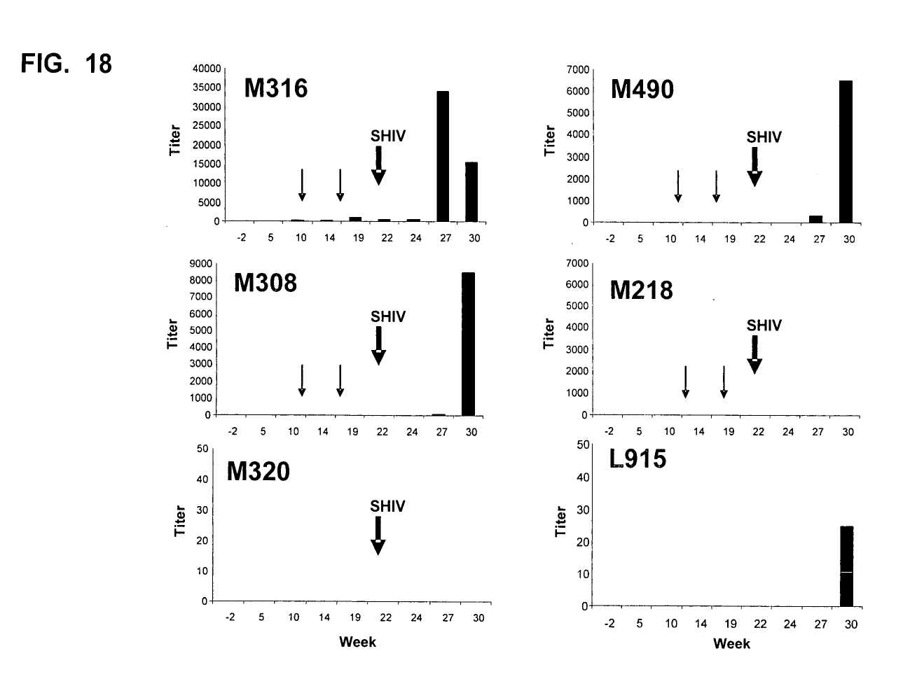

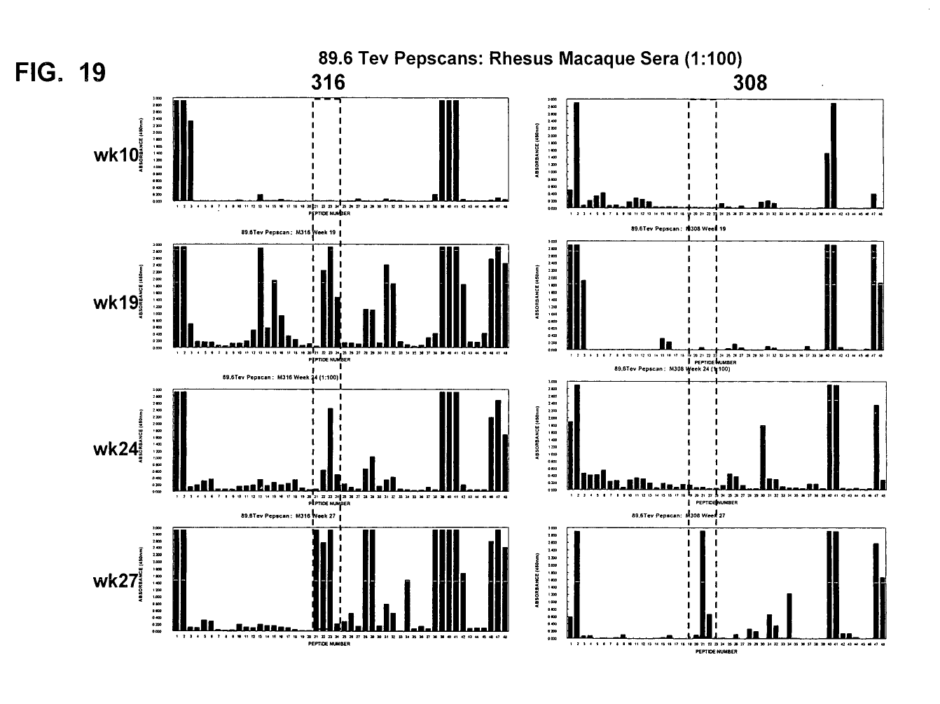

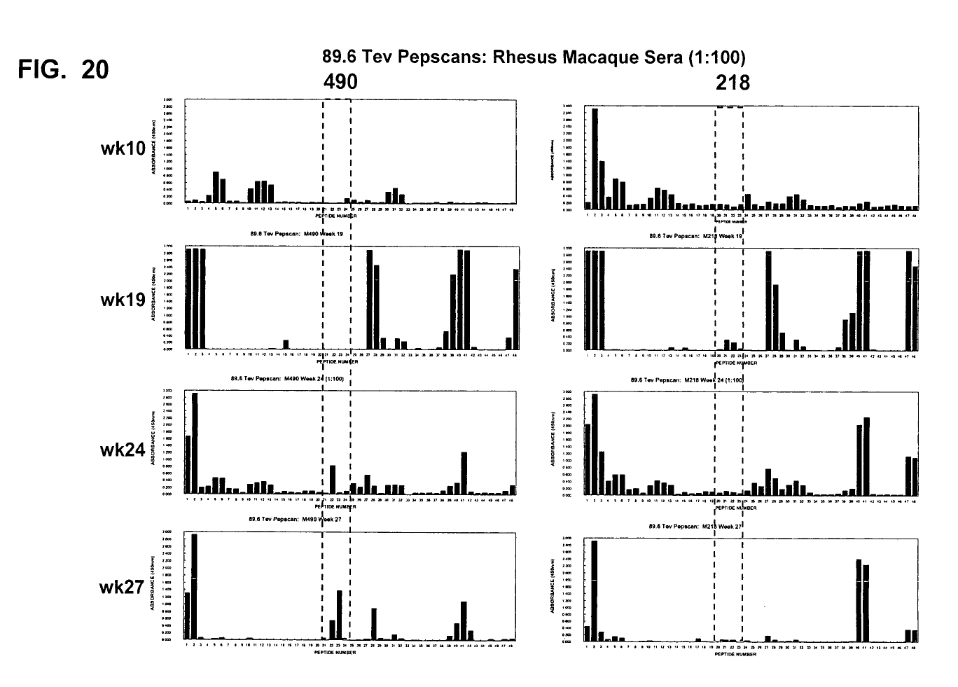

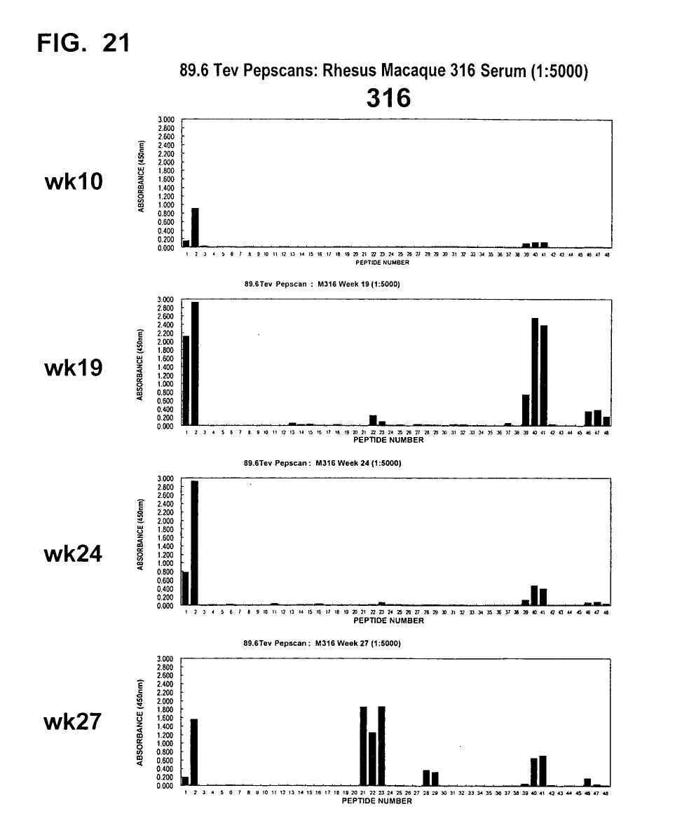

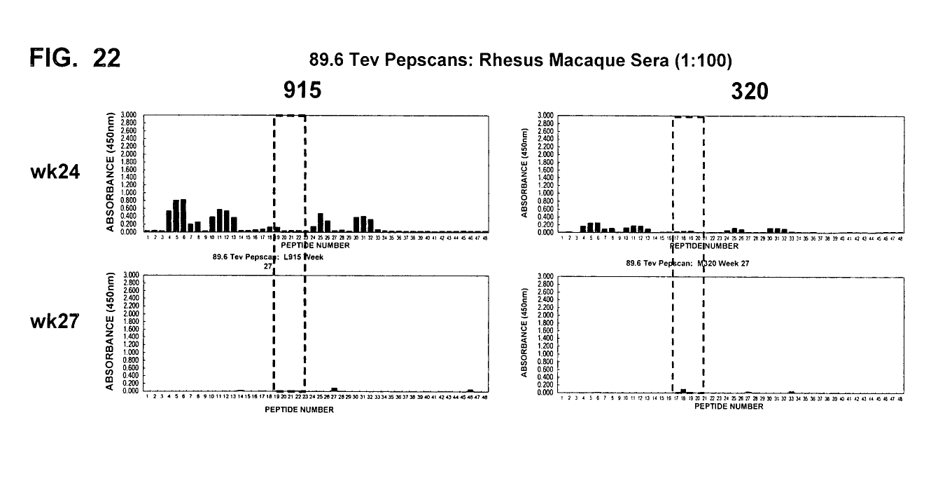

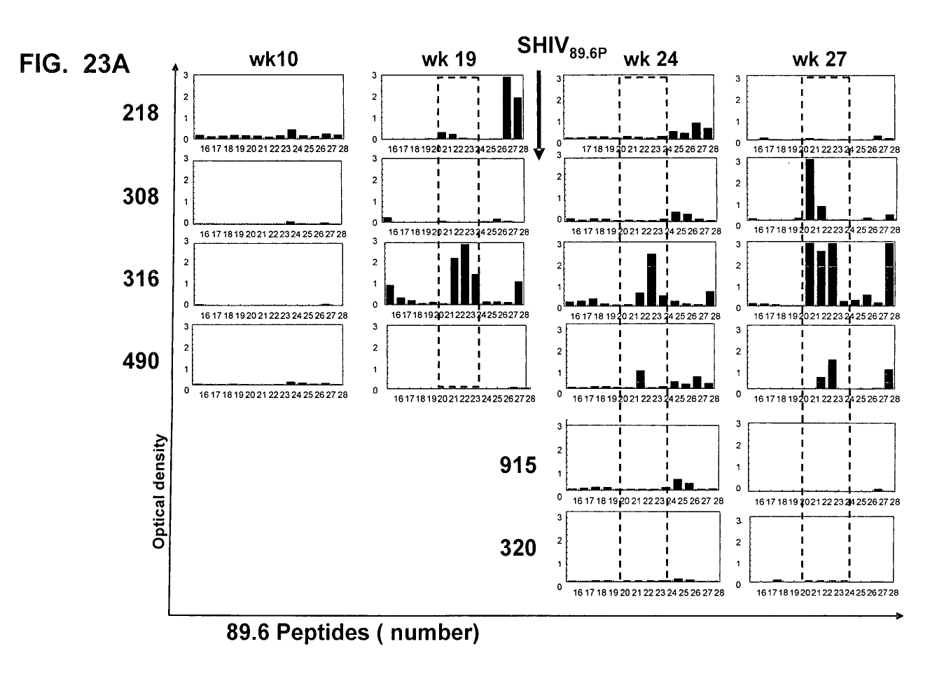

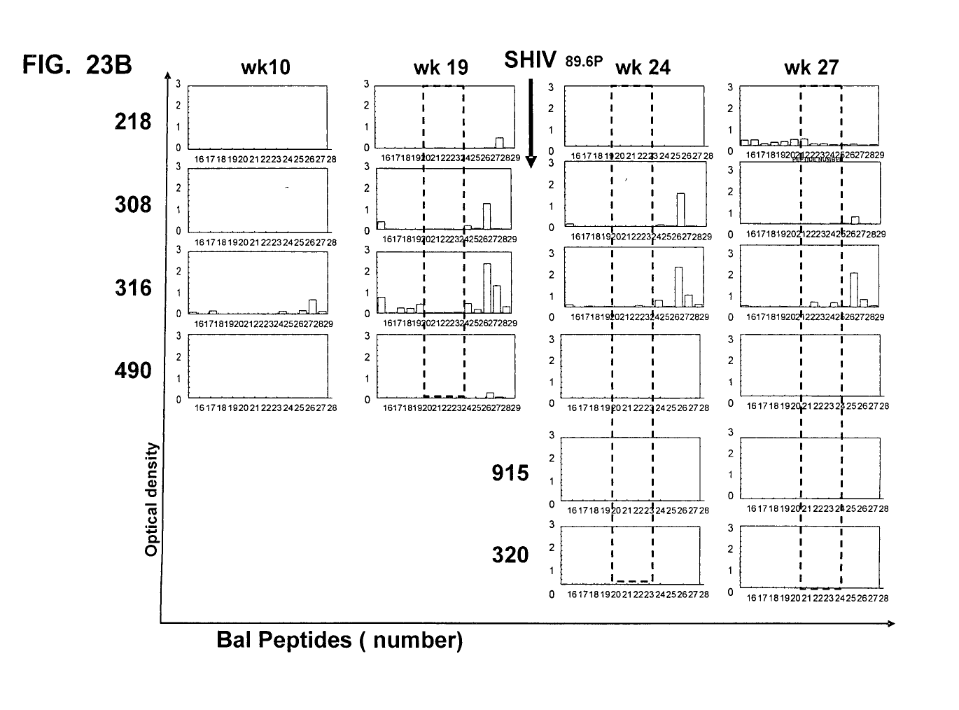

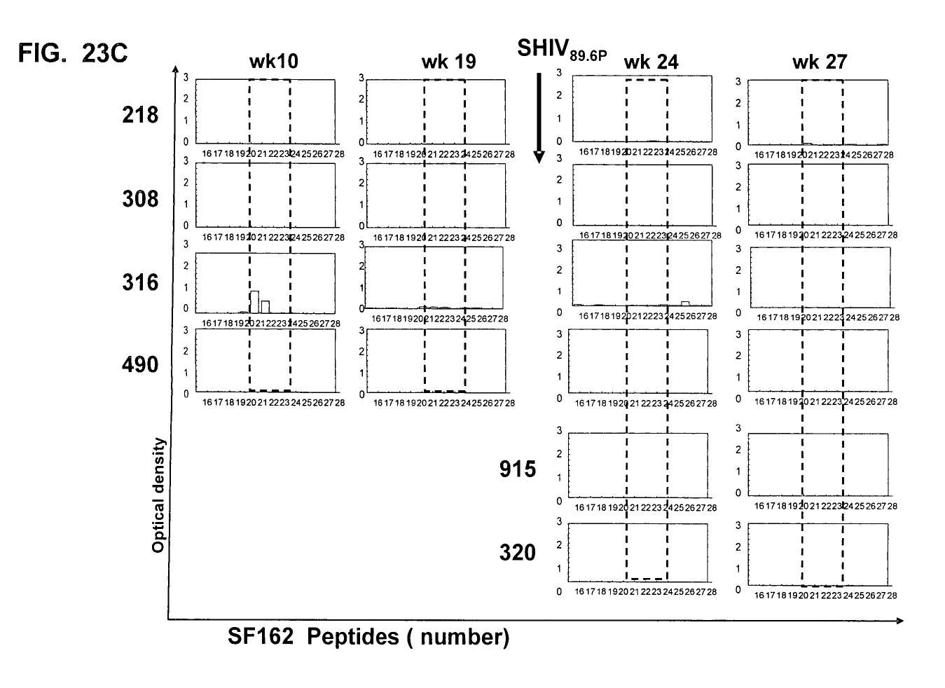

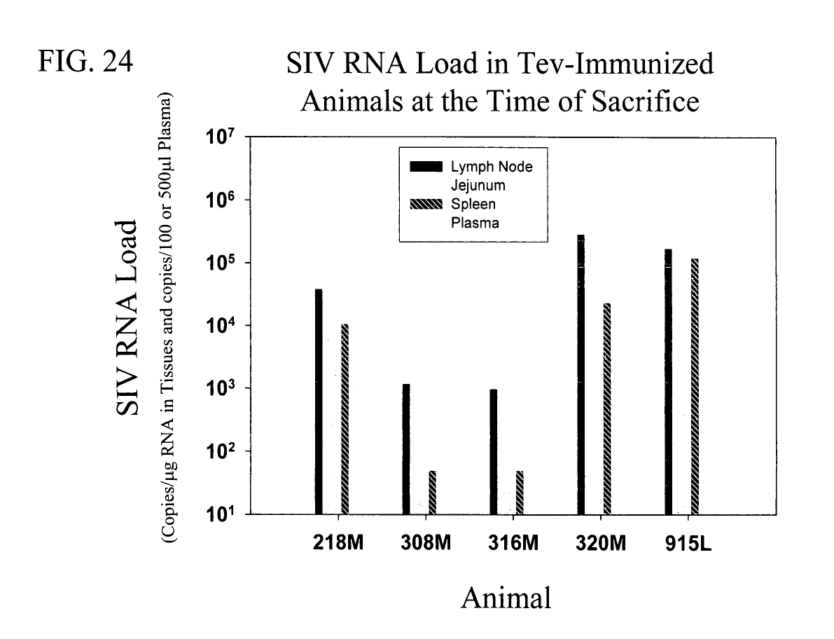

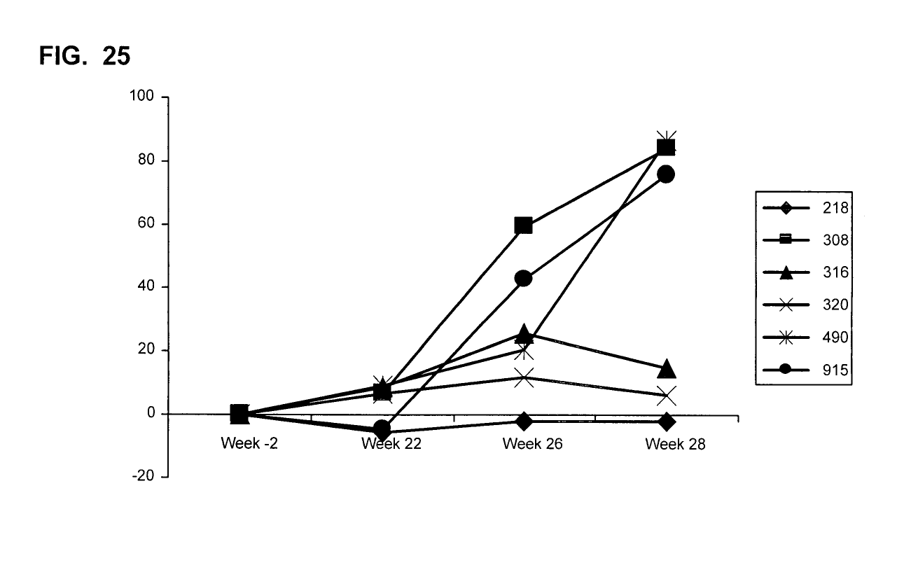

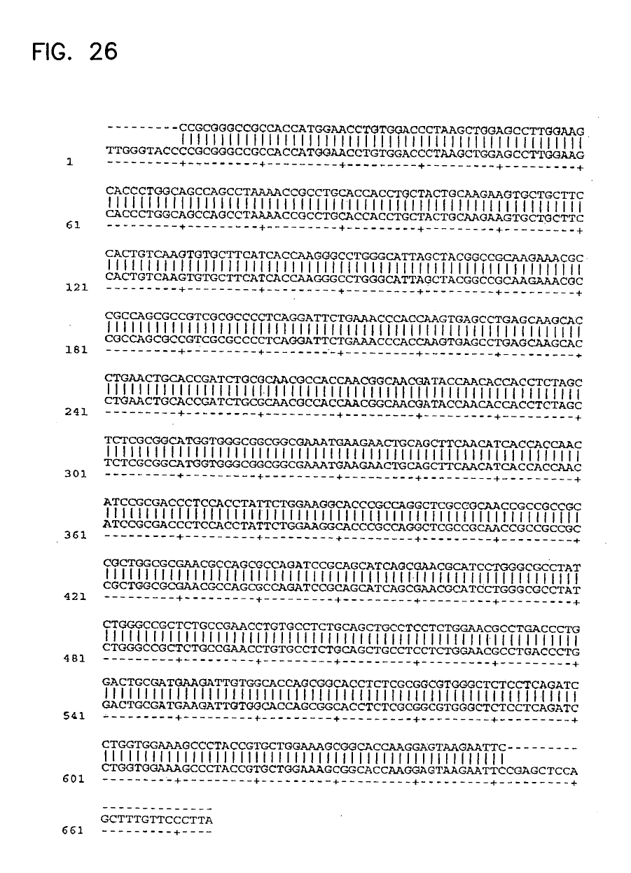

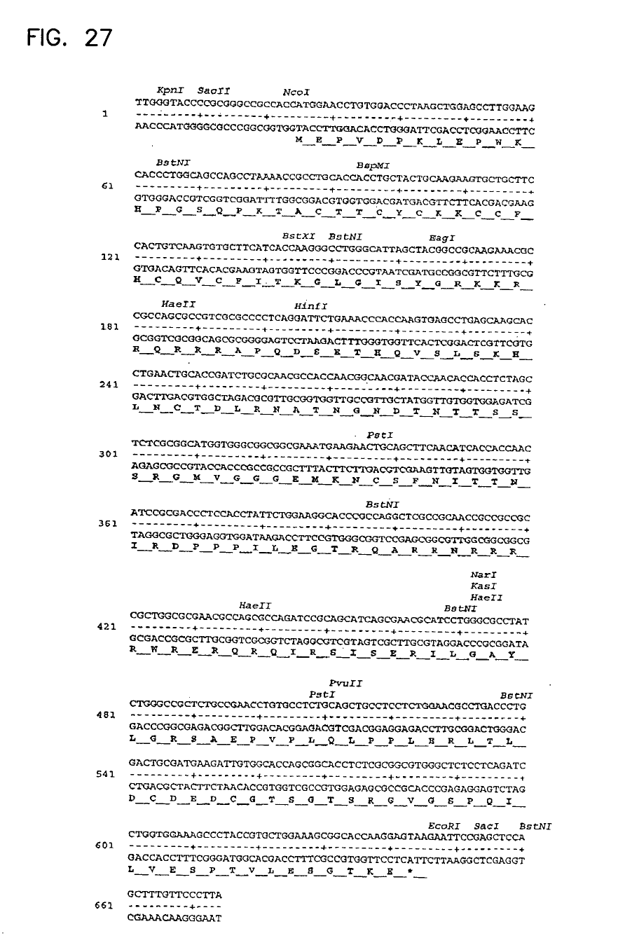

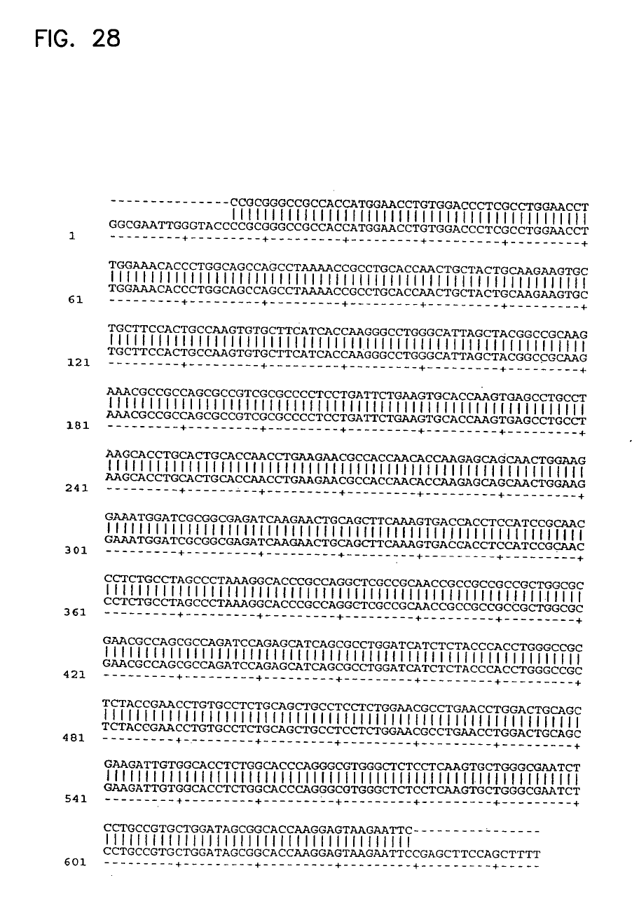

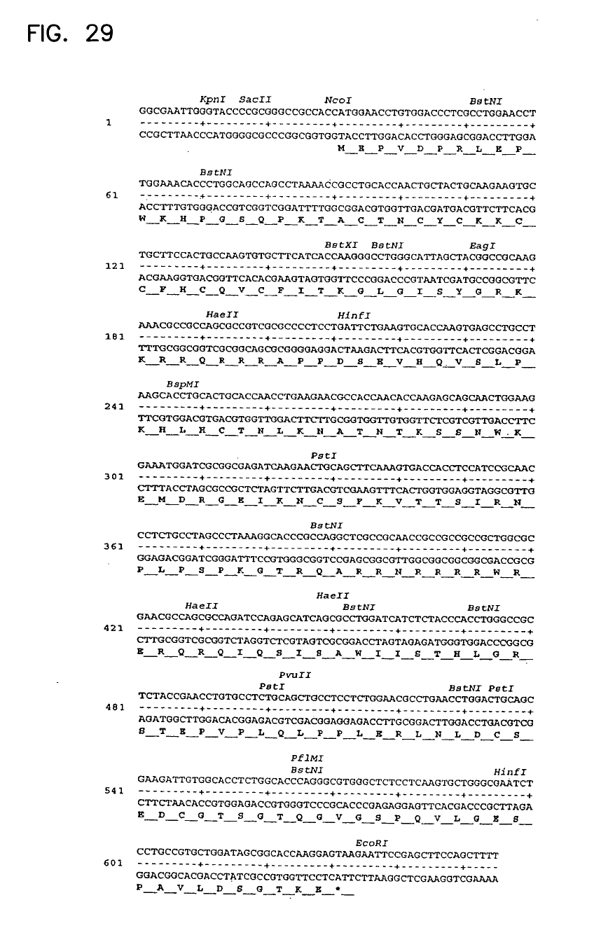

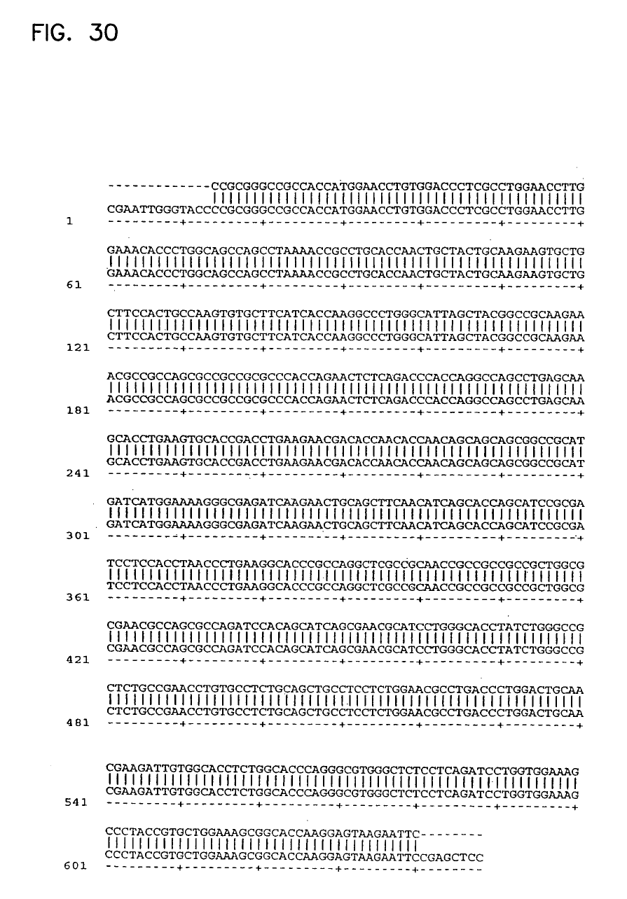

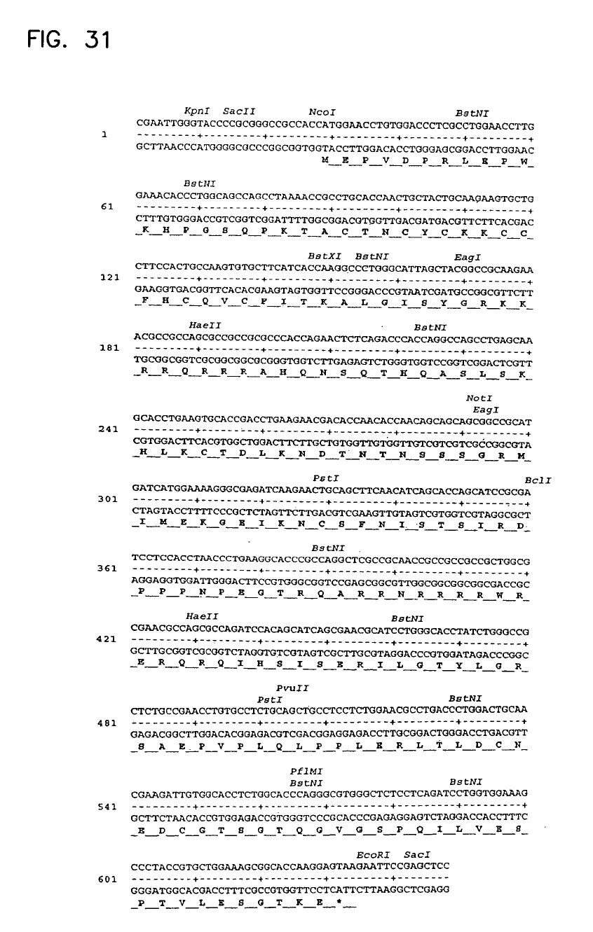

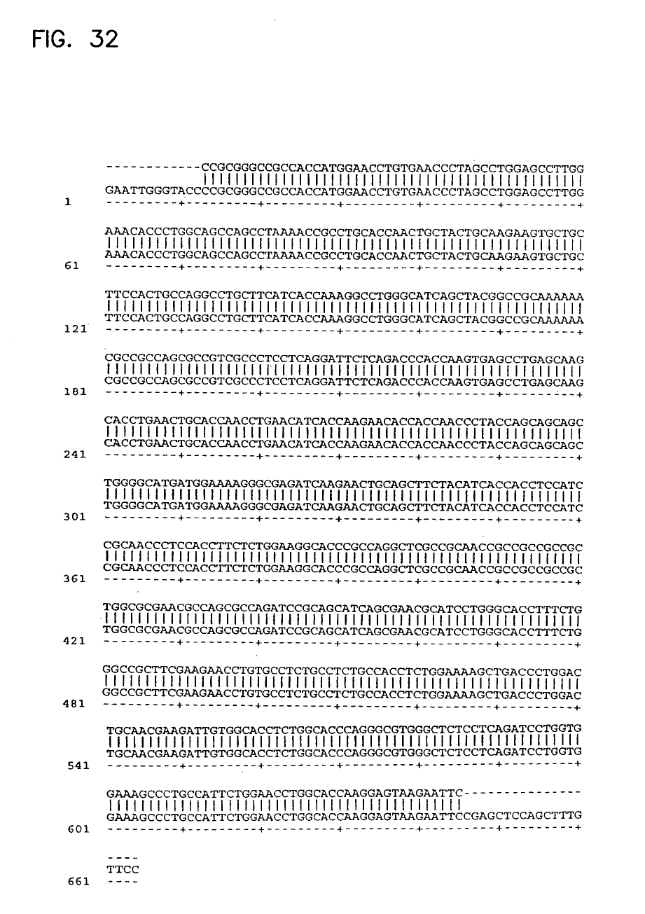

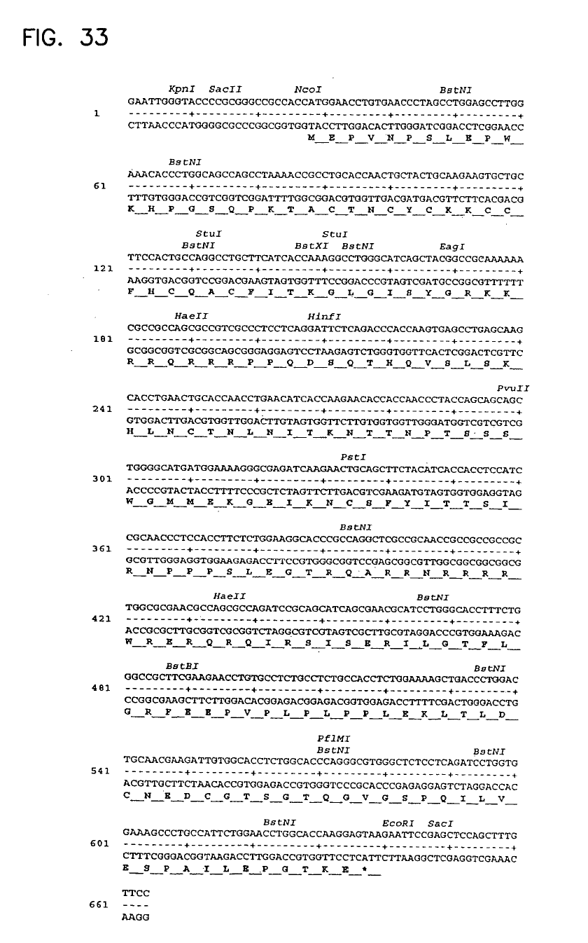

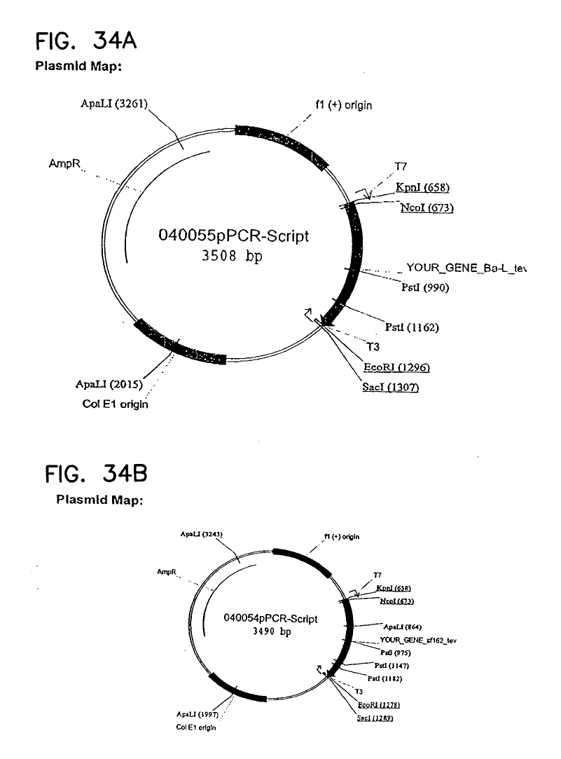

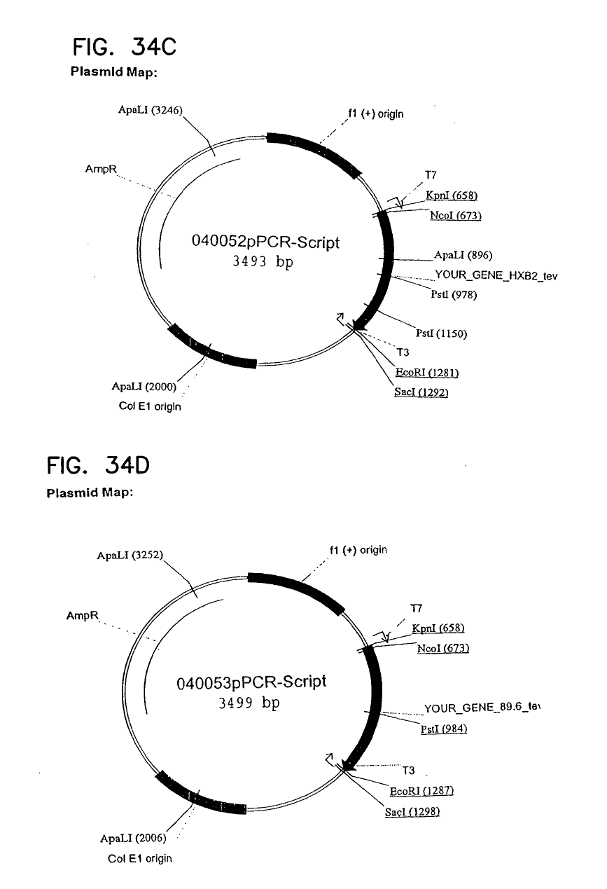

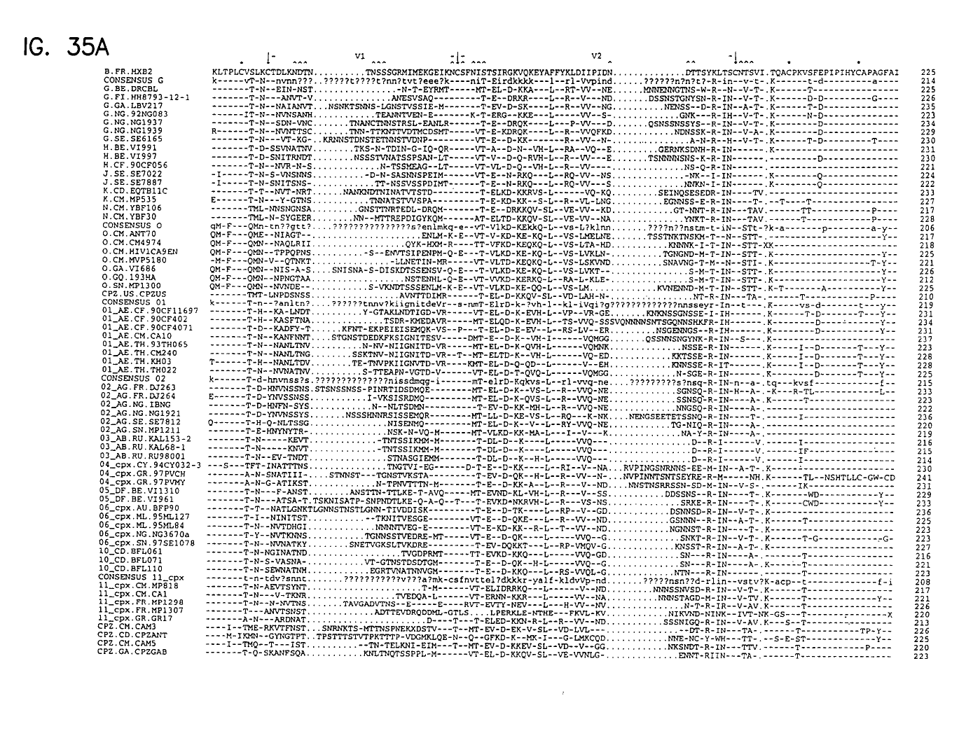

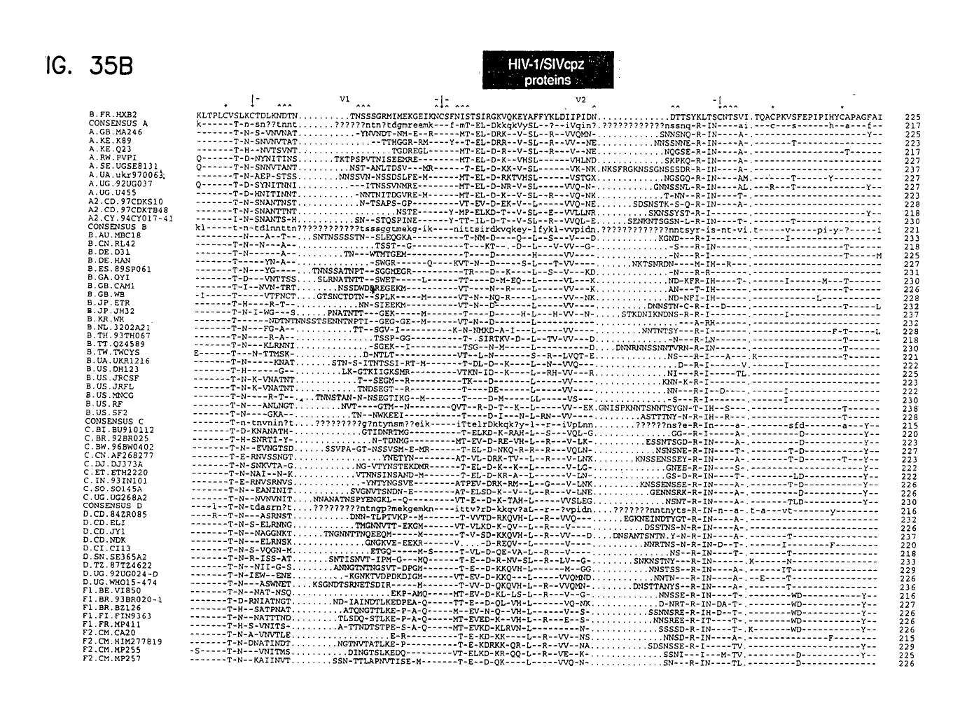

The location of the acceptor and donor sites of exon 6D in HXB2 mRNA is shown in FIGS. 7 and 8 and has been described in Benko et al. cited supra. In one embodiment, the splice acceptor site is found at the 5′ end of exon 6D of the V1 region of envelope protein of HXB2 (e.g. nucleotide 6154 of mRNA of HXB2, see FIG. 7 ) and has a sequence of (A/C/U)AG as underlined in FIG. 7 (SEQ ID NO:3). The donor splice site is found at the 3′ end of exon 6D of the V1 region of envelope protein of HXB2 (e.g. nucleotide 6269 of m RNA of HBX2, see FIGS. 8 and 10) and has a sequence of (A/C/G)AG as underlined in FIG. 8 (SEQ ID NO:4). The nucleotides in the parenthesis represent alternative nucleotides that can be found at the same position. Preferably, the acceptor splice site and donor splice site have a nucleotide sequence comprising a conserved AG or AC. In some embodiments, the conserved nucleotides are flanked 5′ and/or 3′ by one to three nucleotides that contribute to effective splicing. In some embodiments, the acceptor splice site comprises AGG or CAG or UAG (see FIG. 7). In some embodiments, the donor splice site comprises AGG, CAG, or GAG (see FIG. 8).

The locations of donor and acceptor splice sites in the DNA sequence corresponding to the mRNA are shown in FIGS. 7 and 8. The nucleotide numbers refer to the location of the splice sites in the mRNA. The numbering on the DNA sequence does not match that of the splice sites on the mRNA because the numbering of the DNA includes the 5′ LTR sequence. The DNA sequences surrounding the donor and acceptor splice sites for exon 6D from representative HIV strains are aligned. The location of the donor and acceptor site sequences in the mRNA in the corresponding DNA sequences are shown; acceptor site in FIG. 7 and donor in FIG. 8. An examination of the DNA sequences at the location of the splice site sequences indicates these sequences are conserved among many different isolates, especially the donor site. One of skill in the art can identify additional acceptor splice sites and donor splice sites by aligning HIV-1 DNA sequences from the location surrounding the splice site sequences against the reference HXB2 sequence as shown in FIGS. 7 and 8.

In other embodiments, the anti-sense RNA is complementary to the acceptor splice site and about 20 to about 70 nucleotides upstream of the acceptor splice site that contribute to effective splicing. In other embodiments, the anti-sense RNA is complementary to the donor splice site and about 20 to about 70 nucleotides downstream of the donor splice site that contribute to effective splicing. In other embodiments, the anti-sense RNA is complementary to the donor or acceptor splice site and about 20 to about 70 nucleotides on either side of the site.

Antisense RNA may be prepared by any method known in the art for synthesis of DNA and RNA molecules. These methods include techniques for chemically synthesizing oligoribonucleotides, such as solid phase phosphoramide chemical synthesis and recombinant techniques. Antisense RNA may be generated by in vitro or in vivo transcription of DNA sequences. U.S. 20030220287. DNA sequences encoding the antisense RNA may be incorporated into a variety vectors known in the art that incorporate suitable RNA polymerase promoters. Alternatively, antisense cDNA constructs that synthesize the antisense RNA molecule constitutively or inducibly may be introduced stably into a cell. As a means of increasing intracellular stability and half-life, modifications may be made to the antisense RNA molecule. Such modifications include, but are not limited, to the addition of flanking sequences of ribonucleotides to the 5′ and/or 3′ end of the molecule.

Antisense constructs are delivered, for example, as an expression plasmid that when transcribed in the cell produces antisense RNA that is complementary to at least a unique portion of the cellular mRNA that encodes p28^{TEV}, preferably the splice acceptor site. In an embodiment, the antisense RNA molecule comprises a ribonucleotide sequence that is complementary to a specific p28^{TEV} mRNA splice junction. In one embodiment, the splice acceptor site is found at the 5′ end of exon 6D of the V1 region of envelope protein of HXB2 (e.g. nucleotide 6154 of m RNA of HXB2, see FIGS. 7) and has a sequence of (A/C/U)AG as underlined in FIG. 7 (SEQ ID NO:3). The donor splice site is found at the 3′ end of exon 6D of the V1 region of envelope protein of HXB2 (e.g. nucleotide 6269 of m RNA of HBX2, see FIGS. 8 and 10) and has a sequence of (A/C/G)AG as underlined in FIG. 8 (SEQ ID NO:4). The nucleotides in the parenthesis represent alternative nucleotides that can be found at the same position. Preferably, the acceptor splice site and donor splice site have a nucleotide sequence comprising a conserved AG or AC. In some embodiments, the conserved nucleotides are flanked 5′ and/or 3′ by one to three nucleotides that contribute to effective splicing. In some embodiments, the acceptor splice site comprises AGG or CAG or UAG (see FIG. 7). In some embodiments, the donor splice site comprises AGG, CAG, or GAG (see FIG. 8).

There is no particular limitation in the length of antisense RNA. See U.S. 20030220287. The length of antisense RNA is dependent upon the number and composition of complementary bases and the accessibility of the target sequence. U.S. 20030220287. In one embodiment, antisense molecules are preferably 12-20 nucleotides. Optimal lengths of anti-sense molecules are known to those of skill in the art. There can be some mismatch in the anti-sense RNA as long as the antisense molecules binds to the target nucleotide sequence under stringent conditions, preferably under moderately stringent conditions and more preferably under highly stringent conditions.

Similarly, p28^{TEV} RNA levels can be depleted by introduction and/or expression of short interfering RNA (siRNA) designed to target the unique splice junctions present in the p28^{TEV} mRNA. siRNA induces gene-specific suppression through sequence specific degradation of homologous gene transcripts. P. Sharp, 1999, Genes & Devlop., 13:139-141; Berstein et al., 2000, RNA, 97:4985; U.S. 20030220287. For example, the antisense RNA strand of the siRNA is complementary to a portion of the nucleic acid sequence including the splice acceptor site or splice donor site unique to p28^{TEV}.

In one embodiment, the splice acceptor site is found at the 5′ end of exon 6D of the V1 region of envelope protein of HXB2 (e.g. nucleotide 6154 of m RNA of HXB2, see FIGS. 7 and 8) and has a sequence of (A/C/U)AG as underlined in FIG. 7 (SEQ ID NO:3). The donor splice site is found at the 3′ end of exon 6D of the V1 region of envelope protein of HXB2 (e.g. nucleotide 6269 of m RNA of HBX2, see FIG. 8) and has a sequence of (A/C/G)AG as underlined in FIG. 8 (SEQ ID NO:4). The nucleotides in the parenthesis represent alternative nucleotides that can be found at the same position. Preferably, the acceptor splice site and donor splice site have a nucleotide sequence comprising a conserved AG or AC. In some embodiments, the conserved nucleotides are flanked 5′ and/or 3′ by one to three nucleotides that contribute to effective splicing. In some embodiments, the acceptor splice site comprises AGG or CAG or UAG (see FIG. 7). In some embodiments, the donor splice site comprises AGG, CAG, or GAG (see FIG. 8). One of skill in the art can readily identify additional acceptor splice sites and donor splice sites by aligning HIV-1 DNA sequences against a reference sequence HXB2, as shown in FIGS. 7 and 8.

In other embodiments, the anti-sense strand of siRNA is complementary to the acceptor splice site and about 20 to about 70 nucleotides upstream of the acceptor splice site that contribute to effective splicing. In other embodiments, the anti-sense RNA is complementary to the donor splice site and about 20 to about 70 nucleotides downstream of the donor splice site that contribute to effective splicing. In other embodiments, the anti-sense RNA is complementary to the donor or acceptor splice site and about 20 to about 70 nucleotides on either side of the site.

siRNA may be prepared by any method known in the art for synthesis of DNA and RNA molecules, including chemical synthesis and recombinant techniques. U.S. 20040002077; U.S. 20030220287. siRNA may be generated by in vitro or in vivo transcription of DNA sequences.

DNA sequences encoding the siRNA may be incorporated into a variety vectors known in the art that incorporate suitable RNA polymerase promoters. siRNA can be introduced in cultured cells either transiently or via stable expression from tetracycline inducible promoters. Efficient introduction of siRNA molecules into cells in vitro may be performed using a number of technologies, including lipid-based transfection techniques. siRNA can also be expressed in tissues or whole organs using viral expression systems known in the art that incorporate suitable RNA polymerase promoters. Preferably the expression system is introduced into the target cells using a vector that efficiently transfers the expression system. Vectors are selected depending on the type of cell to be transfected. Useful viral vectors include retrovirus vector, adenovirus vector, adeno-associated virus vector, vaccinia virus vector, lentivirus vector, herpesvirus vector, alphavirus vector, EB virus vector, papilloma virus vector, and foamyvirus vector. Useful non-viral vectors include cationic liposome, ligand DNA complex, and gene gun.

siRNA constructs are delivered, for example, as an expression plasmid that when transcribed in the cell produces siRNA comprising an antisense stand that is complementary to at least a unique portion of the cellular mRNA that encodes p28^{TEV} . In an embodiment, the siRNA molecule comprises an antisense strand comprising a ribonucleotide sequence that is complementary to a specific p28^{TEV} mRNA splice junction.

There is no particular limitation in the length of siRNA. See U.S. 20030220287. The siRNA may be 15 to 50 bp long, preferably 15 to 35 bp, and more preferably 21 to 30 bp. The double-stranded RNA portions of siRNAs may contain nonpairing nucleotides due to mismatches, wherein the corresponding nucleotides are not complementary, and/or bulges, wherein the corresponding complementary nucleotide is lacking on one strand. See U.S. 20040002077. Double stranded RNA (dsRNA) may comprise nonpairing nucleotides to the extent the nonpairing nucleotides do not interfere with siRNA formation. There can be some mismatch in the anti-sense RNA strand of the siRNA as long as the antisense strand binds to the target nucleotide sequence under stringent conditions, preferably under moderately stringent conditions and more preferably under highly stringent conditions.

p28^{TEV} production may also be inhibited by modulating pre-mRNA splice site choice. Short, chemically modified antisense oligonucleotides can be targeted to the specific splice junctions present in p28^{TEV}. These chemically modified antisense oligos, including but not limited to 2′-o-methyl-phosphoribonucleotides, morpholinos and PNAs (protein nucleic acids) act in a highly specific fashion by binding to their target sequence without inducing the RNAse H degradation pathway typical for traditional antisense oligonucleotides. Binding of these reagents to the splice site prevents association of the pre-mRNA splicing machinery and thus leads to skipping of the targeted exon. In an embodiment, the targeted exon is exon 6 of p28^{TEV} Anti-sense directed control of splicing is highly specific and efficient and is currently explored in clinical trials (Kalbfuss et al., 2001, JBC, 276:42986-93; Sazani and Kole., 2003, J Clin. Invest., 112:481-6). Oligonucleotide-protein chimeras may be used to target specific splice junctions (Cartegni et al., 2003, Nature Struct. Biol., 10:120-5). The oligonucleotide moiety targets the chimera to the appropriate exon, whereas the protein moiety consisting of minimal SR protein splicing factors stimulates formation of the spliceosome and thus enhances inclusion of the targeted splice site. Splice site junctions may also be targeted in vivo by trans-splicing systems (Deidda et al., Nature Struct. Biol., 2003 12 pp 1499- 1504).

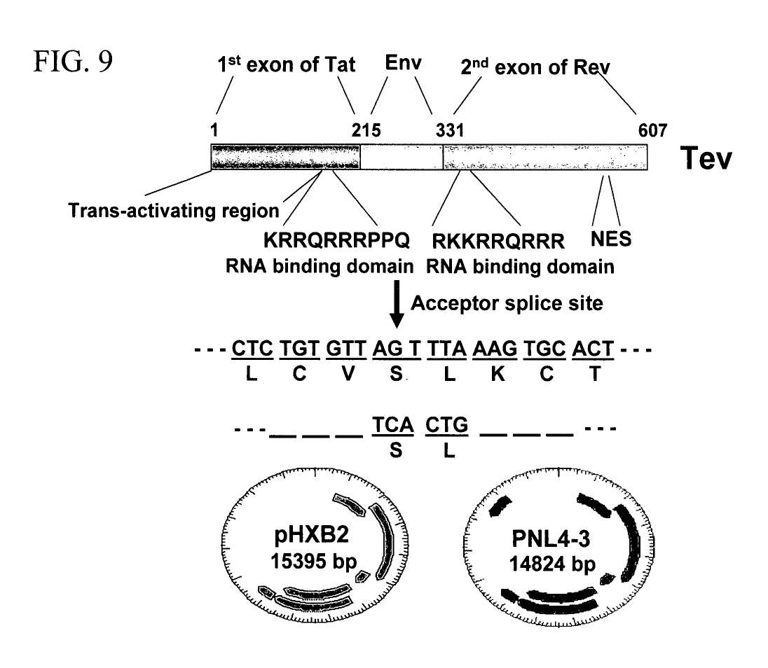

In an embodiment, the antisense oligonucleotide comprises a nucleic acid sequence that is complementary to a specific p28^{TEV} mRNA splice junction. In one embodiment, the splice acceptor site is found at the 5′ end of exon 6D of the V1 region of envelope protein of HXB2 (e.g. nucleotide 6154 of mRNA of HXB2, see FIGS. 7 and 8) and has a sequence of (A/C/U)AG underlined in FIG. 8 (SEQ ID NO:3). The donor splice site is found at the 3′ end of exon 6D of the V1 region of envelope protein of HXB2 (e.g. nucleotide 6269 of m RNA of HBX2, see FIG. 7 and 9) and has a sequence of (A/C/G)AG underlined in FIG. 9 (SEQ ID NO:4). The nucleotides in the parenthesis represent alternative nucleotides that can be found at the same position. Preferably, the acceptor splice site and donor splice site have a nucleotide sequence comprising a conserved AG or AC. In some embodiments, the conserved nucleotides are flanked 5′ and/or 3′ by one to three nucleotides that contribute to effective splicing. In some embodiments, the acceptor splice site comprises AGG or CAG or UAG (see FIG. 7). In some embodiments, the donor splice site comprises AGG, CAG, or GAG (see FIG. 8).

In other embodiments, the anti-sense RNA is complementary to the acceptor splice site and about 20 to about 70 nucleotides upstream of the acceptor splice site that contribute to effective splicing. In other embodiments, the anti-sense RNA is complementary to the donor splice site and about 20 to about 70 nucleotides downstream of the donor splice site that contribute to effective splicing. In other embodiments, the anti-sense RNA is complementary to the donor or acceptor splice site and about 20 to about 70 nucleotides on either side of of the site.

F. Compositions of p28^{TEV} Polypeptides

The p28^{TEV} polypeptides of the present disclosure are useful, for example, in immunogenic compositions, as immunogens for purifying anti-HIV antibodies from sera, for identifying and/or purifying anti-HIV monoclonal antibodies, in a method to screen for HIV, and in a method to identify antagonists of p28^{TEV} polypeptides.

The p28^{TEV} polypeptides of the disclosure can be employed in immunogenic compositions. Immunogenic compositions are useful to elicit p28^{TEV} antibodies and/or to inhibit HIV viral levels. These antibodies are useful, for example, in diagnostic assays to screen for HIV infection and as therapeutic antagonists of p28^{TEV} activity.

The immunogenic composition preferably comprises at least one isolated and/or purified p28^{TEV} polypeptide or immunogenic fragment thereof and more preferably, comprises a mixture of naturally occurring p28^{TEV} polypeptides from at least about 2 to about 6 different HIV-1 clades, more preferably about 3 to about 6 different HIV-1 clades. Because of the variability of the V1 portion of p28^{TEV}, it may be necessary to include p28^{TEV} polypeptides from many different clades.

In some embodiments, the immunogenic composition comprises at least one isolated and/or purified naturally occurring polynucleotide encoding a p28^{TEV} polypeptide or p28^{TEV} polypeptides isolated from infected individuals at various times post infection. Naturally occurring HIV isolated from an individual can be cloned and polynucleotides encoding p28^{TEV} isolated and characterized. The p28^{TEV} polynucleotides or polypeptides so isolated can be utilized to prepare a specific therapeutic or immunogenic composition for an individual.

In some embodiments, an immunogenic composition can comprise at least one V1 peptide from the V1 region of p28^{TEV}, preferably corresponding to amino acids 75-103 of p28^{TEV} of 89.6. In some embodiments, the peptide from the V1 region corresponds to one or more of the peptides 20-24. In some embodiments, the peptide comprises a sequence of:

20 NLNITKNTTNPTSSS, (SEQ ID NO:136)
21 TKNTTNPTSSSWGMM, (SEQ ID NO:137)
22 TNPTSSSWGMMEKGE, (SEQ ID NO:138)
23 SSSWGMMEKGEIKNC, (SEQ ID NO:139)
or
24 GMMEKGEIKNCSFYI. (SEQ ID NO:140)

In other embodiments, the V1 polypeptide comprises an amino acid sequence selected from the group consisting of:

SSSWGMMEKGE, (SEQ ID NO:152)
SSSRGMVGGGE, (SEQ ID NO:153)
SSNWKEMDRGE, (SEQ ID NO:154)
and
SSSGRMIMEKG. (SEQ ID NO:155)

In other embodiments, a V1 consensus sequence comprises a formula of contiguous amino acids comprising SSSX₄X₅MX₇X₈X₉GE (SEQ ID NO:156); wherein X4 is W, R, or G; X5 is G, K, or R; X7 is M, V or I; X8 is E,G,D, or M; and X9 is K,G,R, or E. These peptides can be used to generate or select antibodies specific for p28^{TEV} polypeptide. In some embodiments, the immunogenic composition comprises one or more V1 sequences from a HIV isolate of a specific clade. In some embodiments, consensus sequences of the V1 region for each clade are included in the immunogenic composition.

In other embodiments, an isolated and purified V1 polypeptide comprises at least one V1 consensus sequence of a clade as shown in FIG. 35. In some embodiments, an isolated V1 consensus sequence comprises

for clade A:

(SEQ ID NO:164)

CSNX₃X₄NNTX₈X₉X₁₀NTNX₁₄TDGMREEKNC
for clade B:

(SEQ ID NO:165)

CTDLNNTNX₉X₁₀TSSSGGTMEKGEIKNC
for clade C:

(SEQ ID NO:166)

CTNVNINX₇TX₉X₁₀GX₁₂NTYNSMX₁₉X₂₀EIKNC
for clade D:

(SEQ ID NO:167)

CTDASRNX₈TX₁₀X₁₁NTNGPX₁₇MEKGEMKNC
for clade G:

(SEQ ID NO:168)

CTNVNNX₇X₈X₉X₁₀X₁₁TX₁₃NNX₁₆TVTX₂₀EEEKNC
for clade O:

(SEQ ID NO:169)

CTNX₄X₅GTTX₉X₁₀X₁₁X₁₂X₁₃X₁₄X₁₅X₁₆X₁₇X₁₈ENLMKQC;

wherein an X amino acid is any of the 20 naturally occurring amino acids.

The p28^{TEV} polypeptides are preferably present in an immunogenic effective amount. An immunogenic effective amount is an amount of P28^{TEV} polypeptide that induces an immune response in an animal. The actual amount of the polypeptide may vary depending on the animal to be immunized, the route of administration and adjuvants. Immunogenic dosages can be determined by those of skill in the art. The immune response may be indicated by T and/or B cell responses. Typically, the immune response is detected by the presence of antibodies that specifically bind to a particular p28^{TEV} polypeptide. Methods of detecting antibodies to p28^{TEV} polypeptides are known to those of skill in the art and include such assays as ELISA assays, ELISPOT assays, western blot assays, and competition assays.

In one embodiment, animals are immunized with the antigen, immunogenic conjugates, or derivatives by combining, e.g., 100 μg or 5 μg of the protein or conjugate with 3 volumes of Freund's complete adjuvant and injecting the solution intradennally at multiple sites. One month later the animals are boosted with 1/2 to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

The compositions of the disclosure also include a carrier. Carriers include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ polyethylene glycol (PEG), and PLURONICS™.

The polypeptides of the disclosure can be administered orally or parentally, including subcutaneous injection, intravenous, intramuscular, intrasternal or infusion techniques, in dosage unit formulations containing conventional non-toxic pharmaceutically acceptable carriers, adjuvants or vehicles. Compositions of the disclosure can be in the form of suspensions or tablets suitable for oral administration or sterile injectable preparations, such as sterile injectable aqueous or oleagenous suspensions.

For administration as injectable solutions or suspensions, the compositions can be formulated according to techniques well-known in the art, using suitable dispersing or wetting and suspending agents, such as sterile oils, including synthetic mono- or diglycerides, and fatty acids, including oleic acid.

G. Antibodies

The p28^{TEV} polypeptides of the disclosure are immunogenic and elicit anti-p28^{TEV} polypeptide antibodies. The antibodies have many uses, including purifying p28^{TEV} polypeptide of the disclosure, detecting HIV, targeting HIV infected cells, and/or inhibiting viral levels. The antibodies may be polyclonal or monoclonal antibodies.

The antibody may be used to detect HIV-infection in a fluid or tissue from a subject. The antibody typically will be labeled with a detectable moiety including, but not limited to, a fluorescent label, a radioisotope, or an enzyme-substrate label. The label may be indirectly conjugated with the antibody. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten (e.g., digoxin) and one of the different types of labels mentioned above is conjugated with an anti-hapten antibody (e.g., anti-digoxin antibody).

In another embodiment of the disclosure, the antibody does not need to be labeled. The antibody is detected using a labeled antibody that binds to the first antibody.

Antibodies to p28^{TEV} can be screened for antagonist activity. Preferably, antibodies to the p28^{TEV} polypeptide inhibit HIV viral levels. An antagonist antibody would be screened to determine if there was decrease of viral infectivity or viral load in infected cells expressing p28^{TEV}. Alternatively, the antibodies could be screened for effects on the p28^{TEV} transcriptional activity. In another embodiment, antibodies are selected that participate in antibody dependent cytotoxicity of HIV infected cells.

In some embodiments, the antibody specifically binds to the V1 region of p28^{TEV}. In some embodiments, antibodies specific for V1 region of p28^{TEV} do not crossreact with Tat or Rev region of p28^{TEV}. These antibodies may be useful as therapeutic agents or as targeting agents in combination with an antagonist such as siRNA or cytotoxic agents.

In an embodiment, the antibodies specifically bind p28^{TEV} and do not crossreact with Tat and/or Rev. Screening methods for identifying antibodies that bind to p28^{TEV} and do not bind to Tat and/or Rev are known to those of skill in the art and include competitive binding assays and the like. Preferably, the antibodies bind p28^{TEV} with high affinity.

In a specific embodiment, antibodies to p28^{TEV} can bind to the V1 region of p28^{TEV} In some embodiments, the antibodies bind to one or more of the following peptides or variants thereof:

(20) NLNITKNTTNPTSSS, (SEQ ID NO:136)
(21) TKNTTNPTSSSWGMM, (SEQ ID NO:137)
(22) TNPTSSSWGMMEKGE, (SEQ ID NO:138)
(23) SSSWGMMEKGEIKNC, (SEQ ID NO:139)
or
(24) GMMEKGEIKNCSFYI. (SEQ ID NO:140)

In other embodiments, the V1 polypeptide comprises an amino acid sequence selected from the group consisting of:

SSSWGMMEKGE, (SEQ ID NO:152)
SSSRGMVGGGE, (SEQ ID NO:153)
SSNWKEMDRGE, (SEQ ID NO:154)
and
SSSGRMIMEKG. (SEQ ID NO:155)

In other embodiments, a V1 consensus sequence comprises a formula of contiguous amino acids comprising SSSX₄X₅MX₇X₈X₉GE (SEQ ID NO:156); wherein X4 is W, R, or G; X5 is G, K, or R; X7 is M, V or I; X8 is E,G,D, or M; and X9 is K,G,R, or E.

In other embodiments, an isolated and purified V1 polypeptide comprises at least one V1 consensus sequence of a clade as shown in FIG. 35. In some embodiments, an isolated V1 consensus sequence comprises

for clade A:

(SEQ ID NO:164)

CSNX₃X₄NNTX₈X₉X₁₀NTNX₁₄TDGMREEKNC
for clade B:

(SEQ ID NO:165)

CTDLNNTNX₉X₁₀TSSSGGTMEKGEIKNC
for clade C:

(SEQ ID NO:166)

CTNVNINX₇TX₉X₁₀GX₁₂NTYNSMX₁₉X₂₀EIKNC
for clade D:

(SEQ ID NO:167)

CTDASRNX₈TX₁₀X₁₁NTNGPX₁₇MEKGEMKNC
for clade G:

(SEQ ID NO:168)

CTNVNNX₇X₈X₉X₁₀X₁₁TX₁₃NNX₁₆TVTX₂₀EEEKNC
for clade O:

(SEQ ID NO:169)

CTNX₄X₅GTTX₉X₁₀X₁₁X₁₂X₁₃X₁₄X₁₅X₁₆X₁₇X₁₈ENLMKQC;

wherein an X amino acid is any of the 20 naturally occurring amino acids.

Variants of these peptides have been described herein previously. These peptides can be used to generate or select antibodies specific for p28^{TEV} polypeptide. In some embodiments, the immunogenic composition comprises one or more V1 sequences from a HIV isolate of a specific clade. In some embodiments, consensus sequences of the V1 region for each clade are included in the immunogenic composition.

In another embodiment, the antibodies have differential binding affinity for p28^{TEV} and Tat or Rev. Preferably, antibodies with differential binding affinity have higher affinity for p28^{TEV} as compared to Tat or Rev. Preferably, the binding affinity for p28^{TEV} is at least 100 fold greater than the binding affinity for Tat or Rev in order to allow for the differential detection of p28^{TEV} In a further embodiment, the antibodies with differential binding affinity bind the V1 region of p28^{TEV} at nanomolar concentrations and Tat or Rev at micromolar concentrations. Screening assays and assays for determining the affinity of antibodies are known to those of skill in the art.

In another embodiment, the anti-p28^{TEV} antibodies cross react with Rev or Tat. Antibodies that cross-react with p28^{TEV} and Tat or Rev are useful in method, for example, to screen for HIV infection. In a method for detecting p28^{TEV} in sample, p28^{TEV} is identified by molecular weight. The sample is immunoprecipitated with anti-p28^{TEV} antibody and the proteins in the immunoprecipitate are separated on a SDS gel and identified by molecular weight. p28^{TEV} has a molecular weight of 28,000 D and tat has a molecular weight of 16,000 or 14,000 D and rev has a molecular weight of 19,000 D. Alternatively, a combination of one or more anti-V1 antibodies, anti-tat antibodies or anti-rev antibodies can be utilized to detect p28^{TEV} in samples.

Antibodies may be delivered intracellularly using a transport peptide such as described in Zhao et al., Apoptosis (2003) 8:631, or as described in U.S.20050033033. Antibodies may also be expressed intracellularly in intrabodies. Intracellular antibodies to p28^{TEV} may inhibit viral replication.

H. Production of Antibodies
i. Polyclonal antibodies

Polyclonal antibodies to a p28^{TEV} polypeptide of the disclosure are preferably raised in animals by multiple subcutaneous (sc) or intraperitoneal (ip) injections of the polynucleotide encoding one or more p28^{TEV} polypeptides, or one or more p28^{TEV} polypeptide or fragments thereof, and optionally an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic. anhydride, SOCl₂, or R¹N═C═NR, where R and R¹ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with 1/2 to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

In an alternative embodiment, the animals are immunized with a recombinant adenovirus vector expressing p28^{TEV} polypeptide of the disclosure followed by at least one booster immunization with p28^{TEV} polypeptides of the disclosure.

The polyclonal antibodies generated by the immunizations may undergo a screen for p28^{TEV} antagonist activity. Preferably, antibodies to the p28^{TEV} polypeptide decrease the infectivity of virus produced or inhibit viral levels. In an embodiment, antibodies that specifically bind a p28^{TEV} polypeptide comprising SEQ ID NO: 151 reduce or inhibit HIV viral levels. An antagonist antibody would be screened to determine if there was a decrease or inhibition of viral levels in infected cells expressing p28^{TEV}.

The polyclonal antibodies are also screened by enzyme-linked immunoabsorbent assay (ELISA) to characterize binding. The antigen panel includes all experimental immunogens. Animals with sera samples that test positive for binding to one or more experimental immunogens are candidates for use in monoclonal antibody production. The criteria for selection for monoclonal antibody production is based on a number of factors including, but not limited to, binding patterns against a panel of structured HIV immunogens.

Cross-competition experiments using other mapped Mabs, human sera and peptides can also be performed. Screening methods for identifying antibodies that bind to p28^{TEV}, for example, the V1 region, and do not bind to Tat and/or Rev are known to those of skill in the art and include competitive binding assays and the like. Screening assays and assays for selecting and identifying antibodies that have higher affinity for p28^{TEV} as compared to tat and/or rev are also known to those of skill in the art.
ii. Monoclonal antibodies

Monoclonal antibodies to a p28^{TEV} polypeptide of the disclosure may be made using the hybridoma method first described by Kohler el al., Nature, 256:495 (1975), or may be made by recombinant DNA methods (U.S. Pat. No. 4,816,567).

In the hybridoma method, a mouse or other appropriate host animal, such as a hamster or macaque monkey, is immunized as hereinabove described to elicit lymphocytes that produce or are capable of producing antibodies that will specifically bind to the p28^{TEV} polypeptide used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells are than seeded and grown in a suitable culture medium that preferably contains one or more substances that inhibit the growth or survival of the unfused, parental myeloma cells. For example, if the parental mycloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or enzyme-linked immunoabsorbent assay (ELISA).

After hybridoma cells are identified that produce antibodies of the desired specificity, affinity, and/or activity, the clones may be subcloned by limiting dilution procedures and grown by standard methods (Goding, Monoclonal Antibodies. Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subelones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

The monoclonal antibodies are characterized for specificity of binding using assays as described previously. Antibodies can also be screened for antagonist activity as described previously.
iii) Human or Humanized antibodies

Humanized forms of non-human (e.g., murine) antibodies are chimeric antibodies that contain minimal sequence derived from non-human immunoglobulin. For the most part, humanized antibodies are human immunoglobulins (recipient antibody) in which residues from a CDR of the recipient are replaced by residues from a CDR of a non-human species (donor antibody) such as mouse, rat, rabbit or nonhuman primate having the desired specificity, affinity, and capacity. Useful non-human antibodies are monoclonal antibodies that bind specifically to p28^{TEV}, for example, to the V1 region, and do not substantially crossreact with tat or rev antibodies. Useful non-human antibodies also include antibodies that inhibit or reduce viral levels. Furthermore, humanized antibodies may comprise residues that are not found in the recipient antibody or the donor antibody. These modifications may be made to improve antibody affinity or functional activity. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allerg, Asthma & Immitnol. 1:105-115 (1998); Harris, Biochem. Soc. Transactions 23:1035-1038 (1995); Hurue and Gross, Curr. Op. Biotech 5:428-433 (1994).

Human antibodies that specifically bind and/or antagonize p28^{TEV} transcriptional activity and/or inhibit viral infectivity can also be made using the transgenic mice available for this purpose or through use of phage display techniques.

In a specific embodiment, a humanized antibody is identified that specifically binds to the V1 region of p28^{TEV}. Preferably, the humanized antibody binds to one or more of peptides corresponding to peptides 20-24 of the VI region p28^{TEV} as described herein. In a specific embodiment, a V1 peptide is selected from the group consisting of:

(20) NLNITKNTTNPTSSS, (SEQ ID NO:136)
(21) TKNTTNPTSSSWGMM, (SEQ ID NO:137)
(22) TNPTSSSWGMMEKGE, (SEQ ID NO:138)
(23) SSSWGMMEKGEIKNC, (SEQ ID NO:139)
or
(24) GMMEKGEIKNCSFYI. (SEQ ID NO:140)

In other embodiments, the V1 polypeptide comprises an amino acid sequence selected from the group consisting of:

SSSWGMMEKGE, (SEQ ID NO:152)
SSSRGMVGGGE, (SEQ ID NO:153)
SSNWKEMDRGE, (SEQ ID NO:154)
and
SSSGRMIMEKG. (SEQ ID NO:155)

In other embodiments, a V1 consensus sequence comprises a formula of contiguous amino acids comprising SSSX₄X₅MX₇X₈X9GE (SEQ ID NO:156); wherein X4 is W, R, or G; X5 is G, K, or R; X7 is M, V or 1; X8 is E,G,D, or M; and X9 is K,G,R, or E.
These peptides can be used to generate or select antibodies specific for p28^{TEV} polypeptide. In some embodiments, the immunogenic composition comprises one or more V1 consensus sequences from a HIV isolate of a specific clade. In some embodiments, consensus sequences of the V1 region for each V1 clade are included in the immunogenic composition.

In other embodiments, an isolated and purified V1 polypeptide comprises at least one V1 consensus sequence of a clade as shown in FIG. 35. In some embodiments, an isolated V1 consensus sequence comprises

for clade A:

(SEQ ID NO:164)

CSNX₃X₄NNTX₈X₉X₁₀NTNX₁₄TDGMREEKNC
for clade B:

(SEQ ID NO:165)

CTDLNNTNX₉X₁₀TSSSGGTMEKGEIKNC
for clade C:

(SEQ ID NO:166)

CTNVNINX₇TX₉X₁₀GX₁₂NTYNSMX₁₉X₂₀EIKNC
for clade D:

(SEQ ID NO:167)

CTDASRNX₈TX₁₀X₁₁NTNGPX₁₇MEKGEMKNC
for clade G:

(SEQ ID NO:168)

CTNVNNX₇X₈X₉X₁₀X₁₁TX₁₃NNX₁₆TVTX₂₀EEEKNC
for clade O:

(SEQ ID NO:169)

CTNX₄X₅GTTX₉X₁₀X₁₁X₁₂X₁₃X₁₄X₁₅X₁₆X₁₇X₁₈ENLMKQC;

wherein an X amino acid is any of the 20 naturally occurring amino acids.
iv. Antibody conjugates

The antibodies specific for a p28^{TEV} polypeptide or fragment thereof can be combined with heterologous moieties to provide a detectable label or for targetd delivery of an inhibitory agent.

Detectable labels include radionuclides, biotin, dyes, enzymes, and fluorescent molecules.

Inhibitory agents include cytotoxic agents such as toxins, and siRNA molecules. SiRNA molecules are described herein specific for a p28^{TEV} polypeptide or fragment thereof. Other siRNA molecules can be constructed that are specific for other HIV proteins such as tat, rev or reverse transcriptase.

I. Uses and Methods

The p28^{TEV} polypeptides and/or polynucleotides as described herein can be utilized in immunogenic compositions, diagnostic assays, to develop monoclonal antibodies, and as potential targets for other inhibitory agents.

In one aspect of the disclosure, a method is provided for immunizing a mammal with an immunogenic composition as described herein. A method for inhibiting HIV viral levels comprises administering at least one isolated nucleic acid encoding a p28^{TEV} polypeptide or fragment thereof. In some embodiments, the fragment of the p28^{TEV} polypeptide includes all or a portion of the V1 region. In some embodiments, the immunogenic composition comprises an immunomodulator or adjuvant. In some embodiments, the immunogenic composition may comprise at least one isolated and/or purified p28^{TEV} polypeptide or fragment thereof.

The p28^{TEV} polypeptides are useful to develop antibodies to the p28^{TEV} polypeptides. Methods for developing antibodies have been described herein. Antibodies may be useful in diagnostic assays for detecting the presence of HIV in a biological sample. One aspect of the disclosure provides a method for screening for HIV, comprising contacting a biological sample with at least one anti-p28^{TEV} polypeptide antibody and assaying the biological sample for anti-p28^{TEV} antibody binding of the protein. Antibodies to V1, tat and rev can be used in combination to detect HIV infection. Antibodies are also useful to purify the p28^{TEV} polypeptides or provide for targeted delivery of a cytotoxic agent.

p28^{TEV} polypeptides may be useful in diagnostic or prognostic assays. The presence or absence of an antibody to a p28^{TEV} polypeptide in a biological sample can be determined using standard methods. Alternatively, presence or absence of p28^{TEV} polypeptide in a biological sample can be determined using PCR primers specific for nucleic acids encoding p28^{TEV} polypeptides to amplify any HIV-1 DNA that may be present in the sample.

The disclosure also provides methods for screening for agents that may inhibit or antagonize p28^{TEV} polypeptides. The p28^{TEV} polypeptide identified herein may have a role in regulating HIV infectivity. Novel therapeutics may be identified that can inhibit the expression or activity of these p28^{TEV} polypeptides. One aspect of the disclosure provides a method for screening for agents that inhibit a p28^{TEV} polypeptide comprising expressing a p28^{TEV} polypeptide in a cell or liposome; contacting the cell with an agent and determining whether the agent inhibits the transcriptional activity and/or effect of p28^{TEV} polypeptide on HIV viral levels. The effect of p28^{TEV} polypeptide or inhibition thereof may be quantified by transactivation measured in a Luc assay.

Potential agents that can act to inhibit the effect of p28^{TEV} on HIV-1 production include antagonist antibodies, antisense RNA specific for p28^{TEV}, small interfering RNA (siRNA) approaches, microRNA or methods to modulate pre-mRNA splice site choice. Antagonist antibodies can be prepared and screened as described previously.

p28^{TEV} antagonists or immunogenic compositions of the disclosure can be used in combination with one or more antiviral compositions to treat patients infected with HIV.

Antagonists of p28^{TEV} polynucleotides or polypeptides or immunogenic compositions can be administered prior, concurrent, or subsequent to administration of the one or more antiviral compositions. The antiviral compositions can be nucleoside reverse transcriptase inhibitors, nonnucleoside reverse transcriptase inhibitors, protease inhibitors, fusion inhibitors, or attachment inhibitors. Examples of nucleoside reverse transcriptase inhibitors include, but are not limited to, AZT, ddl (didanosine), ddC (zalcitabine), d4T (stavudine), Abacavir, Tenofovir, Combivir, Trizivir, Emtricitabine, Epzicom, and Truvada. Examples of nonnucleoside inhibitors include, but are not limited to, Nevirapine, Delavirdine, and Efavirenz. Examples of protease inhibitors include, but are not limited to, Saquinavir, Indinavir, Ritonavir, Nelfinavir, Amprenavir, Lopinavir, Atazanavir, and Fosamprenavir. Examples of fusion inhibitors include, but are not limited to, Enfuvirtide.

In an embodiment, one or more p28^{TEV} antagonists or immunogenic compositions of the disclosure is administered in combination with one or more antiviral compositions to a patient 20 infected with HIV-1. The patient can have a viral load that is “undectable” by a viral load test.

Methods for determining viral load are known. The p28^{TEV} antagonists or immunogenic compositions can be administered prior, concurrent, or subsequent to administration of the one or more antiviral drugs.

p28^{TEV} antagonists or immunogenic compositions of the disclosure are useful for decreasing the probability of developing HIV that are resistant to antiviral compositions. Guidelines for treating HIV and preventing development of resistance to therapeutic compositions with combination therapies are known. See, for example, the U.S. Department of Health and Human Services guidelines for medical management of HIV in adult patients, adolescent patients, or pediatric patients at www-aidsinfo-nih-gov/guidelines. In an embodiment, two or more p28^{TEV} antagonists of the disclosure, preferably three or more p28^{TEV} antagonists of the disclosure, are administered to a patient infected with HIV-1 to decrease the probability of the patient developing virus that is resistant to a p28^{TEV} antagonist of the disclosure. In an embodiment, one or more p28^{TEV} antagonists or immunogenic compositions of the disclosure are administered in combination with one or more, preferably two or more, preferably three or more antiviral compositions to a patient infected with HIV-1 to decrease the probability of the patient developing virus that is resistant to any of the antiviral compositions or antagonists of the disclosure in the combination therapy. The antiviral compositions can be a combination of nucleoside reverse transcriptase inhibitors, nonnucleoside reverse transcriptase inhibitors, protease inhibitors, fusion inhibitors, and attachment inhibitors. The p28^{TEV} antagonists or immunogenic compositions can be administered prior, concurrent, or subsequent to administration of the one or more antiviral compositions.

The following examples are provided for illustrative purposes only, and are in no way intended to limit the scope of the present disclosure.

### EXAMPLES

### Example 1

### HIV-1 p28^{TEV} is Expressed in the Nucleolus and Cytoplasm of a Cell

Previous reports concerning expression of p28^{TEV} in infected cells indicated that p28^{TEV} was only found in the nucleolus. Benko et al. cited supra. The HTLV-1 protein p30 II thought to restrict viral replication and contribute to a latent state in HTLV-1 infected cells. p30II is also found only in the nucleolus of infected cells. We investigated the location of expression of p28^{TEV} in HeLa cells transfected with cDNA encoding p28^{TEV}.

Materials and Methods

DNA constructs: A cDNA construct encoding p28^{TEV} was constructed as described in Benko et al cited supra. The cDNA encoding p28^{TEV} was fused to at its C terminus to a sequence encoding green fluorescent protein by cloning into the EcoRI restriction site in pGFP-C3 vector (Promega Corp, Madison, Wis.). The p30II cDNA was subcloned into EGPN3 at the HindIII-EcoRI site so that the green fluorescent protein was fused at the amino terminal end of the protein

Transfections: Transfections were performed using 5 micrograms of DNA into HeLa cells using electrophoresis.

Detection of Expression: Expression of p28^{TEV} and p30 II was detected in cells using confocal microscopy.

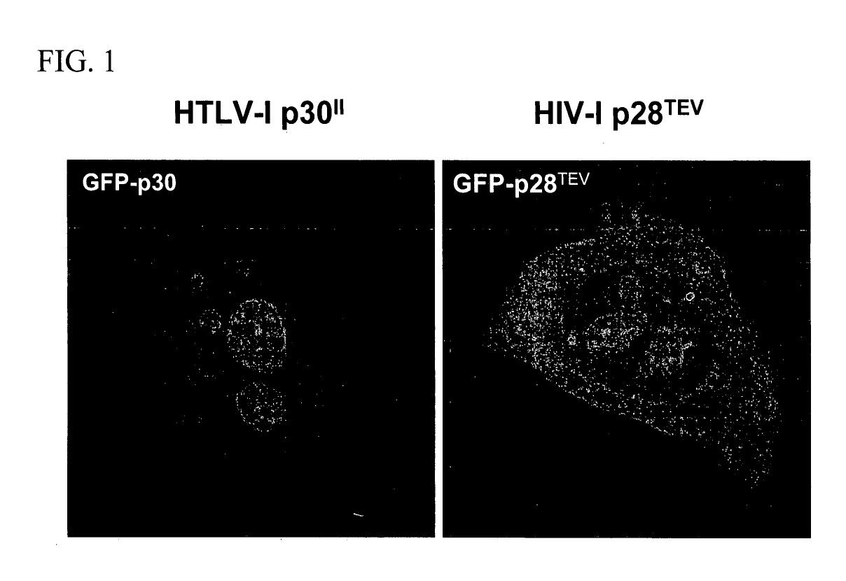

Results As shown in FIG. 1, p28^{TEV} is not only in the nucleolus, as previously reported by Benko et al., 1990, J Virol., 64:2505-2518, but also in the cytoplasm. In contrast, HTLV-1 p30II is located in the nucleolus and nucleoplasm. Like Tat, p28^{TEV} is found in the supernatant (likely released upon cell death) and may be capable of uptake by neighboring cells (data not shown).

### EXAMPLE 2

### Overexpression of HIV-1 p28^{TEV} Inhibits Viral Production

As described previously, HIV-1 infection results in production of multiply spliced mRNAs that encode transcriptional regulatory proteins, Tat and Rev. The function of p28^{TEV} in infected cells has not been described. The p28^{TEV} polypeptide retains both Tat and Rev functions. We investigated the function of p28^{TEV} by coexpressing it with HXB2 provirus in 293T cells.

Construction of DNA Plasmids

To assess the activity of p28^{TEV} polypeptide, we expressed p28^{TEV} in trans simultaneously with the HXB2 provirus. The infectious HIV-1 proviral clone HXB2 was used in all experiments and has been previously described (Fisher et al., 1985, Nature, 316:262-265). The HXB2 clone was obtained from Fisher et al.

The p28^{TEV} construct pNL1.4.6D.7 was constructed by inserting a cDNA encoding p28^{TEV} into a cDNA expression plasmid. Benko et al., 1990, J. Virol., 64:2505-2518; Schwartz et al., 1990, J. Virol., 64:2519-2529. The BssHII-to-BamHI fragment of the cDNA expression plasmid was ligated with the 7.1 kilobase-pair BssHII-to-BamHI fragment of pNL43, which provided the HIV-1 LTR promoter and polyadenylation signal. Benko et al., 1990, J. Virol., 64:2505-2518.

p28^{TEV} polypeptide retains the Tat activation/elongation domain. Therefore, p28^{TEV} polypeptide activity was assessed with the HIV-1-Luc reporter gene. The HIV-1-Luc reporter construct was prepared as described by M. R. Smith & W. C. Greene, 1990, Genes Dev., 4:1875-1885.

We performed all experiments adding RT-LK as an internal control for efficiency of transfection. The RT-LK vector was obtained form Promega Corp.

Luc Assay

HIV p28^{TEV} retains the Tat activation/elongation domain. Therefore, p28^{TEV} polypeptide activity was assessed with the HIV-1-Luc reporter gene. 293T human kidney cells were maintained in Dulbecco's Modified Eagle Medium supplemented with 5% Fetal Bovine Serum, 1% L-glutamine, and 1% Penicillin-Streptomycin (Invitrogen, Grand Island, N.Y.). The 293T cells were transformed using Effectene (Qiagen, Valencia, Calif.). 0.5, 1, 5, and 10 micrograms of DNA were transfected. Transfection efficiencies were normalized using RL-TK.

The infectivity assay is expressed as luciferase units as the virus is applied to Hela cells expressing on the cell surface the HIV-1 receptors CCR5 and CD4 and CXCR4 and carry an HIV-1 LTR luciferase construct that can be activated following viral infection and production of the viral transactivator, Tat.

Viral Production Assay

Viral production was assessed 24 hours after transfection by quantifying the amount of HIV-1 p24 in the supernatant of cell cultures by enzyme-linked immunosorbent assay (ELISA) or intracellular expression of p24 by Western blotting.

ELISA was used to measure p24 in the supernatant. p24 was measured 24 hrs after transfection with the Retro-Tek HIV-1 p24 antigen ELISA kit (ZeptoMetrix Corp., Buffalo, N.Y.). Ninety-six-well Nunc Maxisorp microtiter plates (Fisher Scientific Co., Pittsburgh, Pa.) were coated at 4° C. overnight with monoclonal antibody specific for p24 core protein. The plates were washed 6 times with 1X plate wash buffer. Two hundred microliters of supernatant diluted with 25 microliters of lysing buffer was added to each well and the plate was incubated for 2 h at 37 ° C. The plate was washed as described above and than incubated with HIV-1 p24 detector antibody for 1 h. The plates were washed four times with 1X plate wash buffer, developed with streptavidin-peroxidase working solution and substrate working solution for 30 min, and the absorbance was measured at 450 mn with a Victor multilabel counter spectrophotometer (PerkinElmer, Boston, Mass.).

Western blots were performed as previously described. Sambrook et al., Molecular Cloning: A Laboratory Manual, New York: Cold Spring Harbor Press, 1989. Transfected cells were lysed with 2% deoxycholic acid, 2% SDS, 2% Triton X, 0.3M NaCL, and IM Tris pH 7.4 (RIPA buffer) with 1 ug/ml aprotonin, 1 mM DTT, and 1 mM PMSF. Forty to fifty micrograms total protein was transferred to nitrocellulose membrane (BioScience, Keene, N.H.) in 1X Tris/Glycine buffer (Bio-Rad, Hercules, Calif.) at 85V for 2 hrs at 4° C. The membrane was blocked with 5% milk in 0.1M NaCl, 0.01M Tris-HCL pH 7.5 and 0.001M EDTA (TNE buffer) for 30 min at RT. The membrane was rinsed twice and incubated with a monoclonal antibody to HIV-1 p24 (Advanced BioScience Laboratories, Inc., Rockville, Md.) in TNE buffer at 1:250 o/n at 4° C. The membrane was washed for 1 hr in TNE buffer+5% Triton X-100 with vigorous shaking and incubated with HRP-linked goat anti-mouse IgG (Santa Cruz Biotechnology, (Santa Cruz, Calif.) in INE buffer at 1:8000 for 1 hr. The membrane was rinsed and washed as described above and visualized using a commercial ECL detection kit (Amersham Biosciences, Buckinghamshire, England).

Results

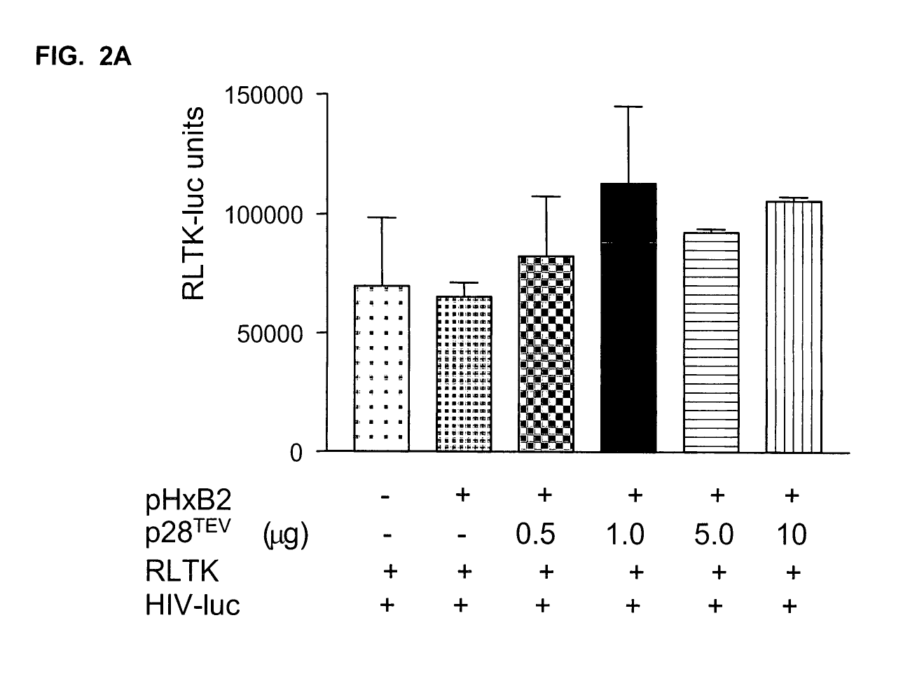

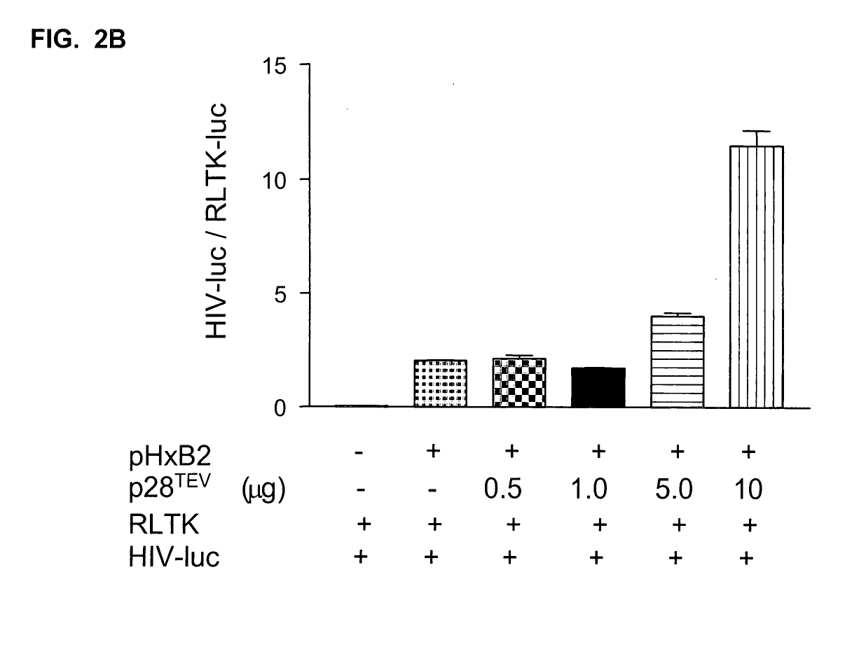

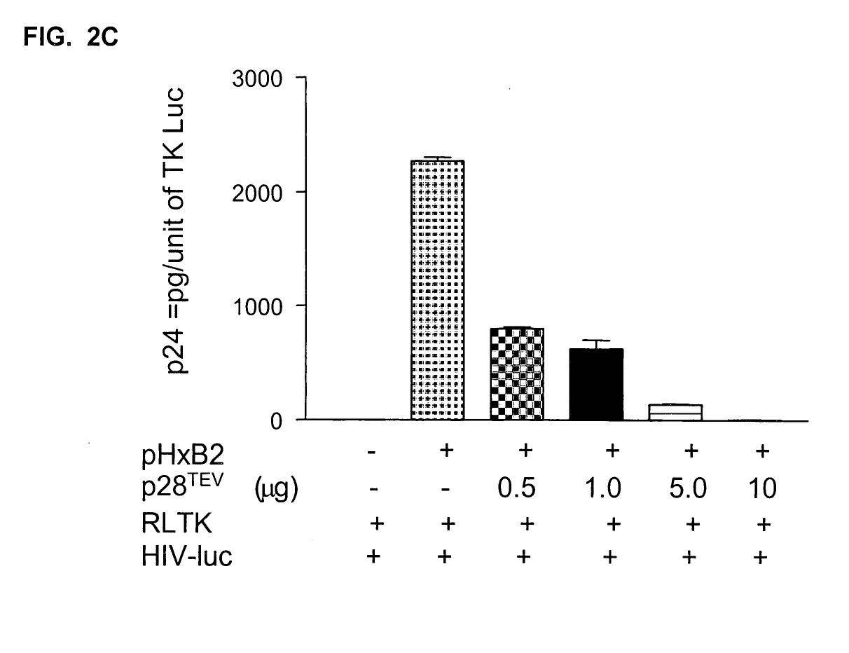

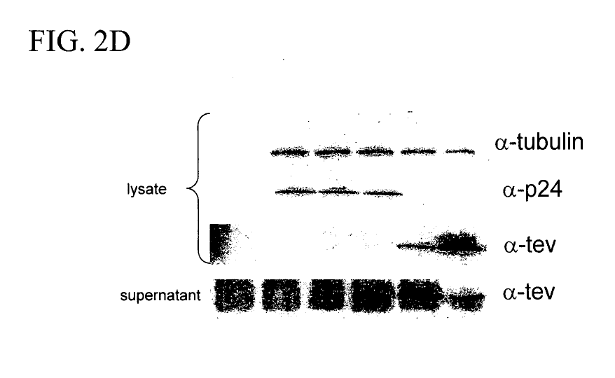

As shown in FIG. 2A, the efficiency of transfection was comparable in all experiments. The results also show that transfecting cells with increasing amounts of p28^{TEV} increased Luc from the HIV-1-Luc reporter gene (FIG. 2B), indicating that expression of p28^{TEV} resulted in increasing tat activity. Despite an increase in Tat activity of the p28^{TEV} polypeptide (FIG. 2B), viral production as measured by HIV-1 p24 in the supernatant was surprisingly decreased (FIG. 2C). We also observed a decrease in intracellular production of p24 and an increase in tev by Western blot (FIG. 2D) using antibodies directed to Rev. The size of Tev was differentiated by that of Rev using molecular weight markers.

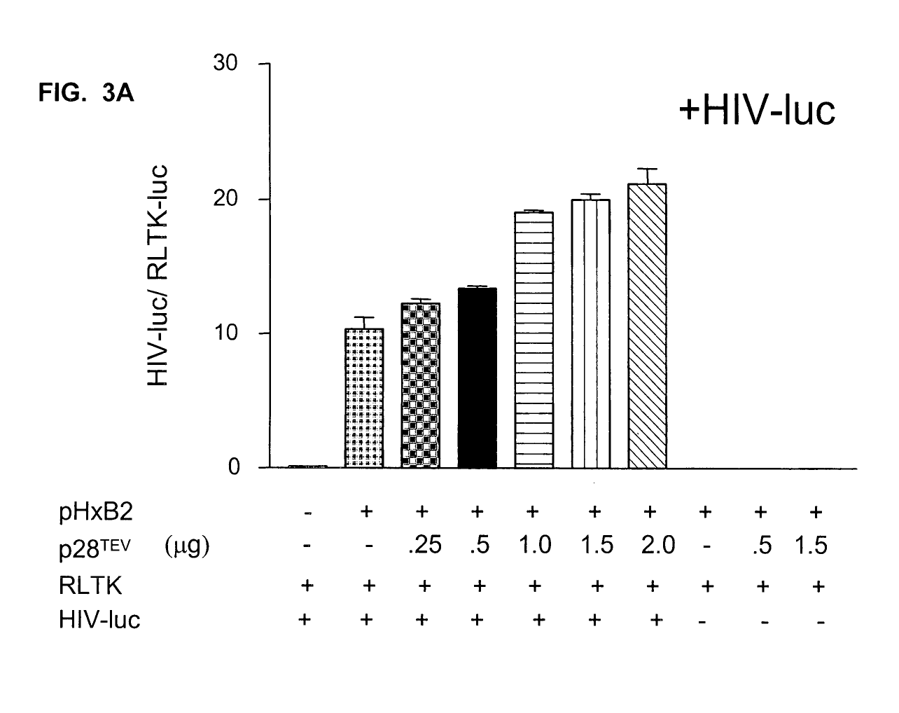

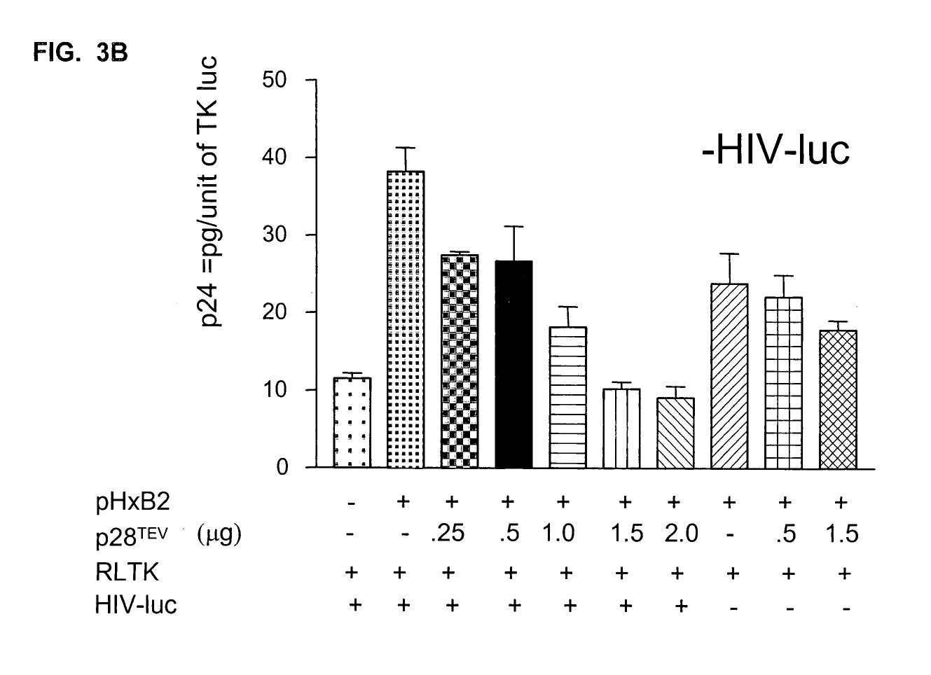

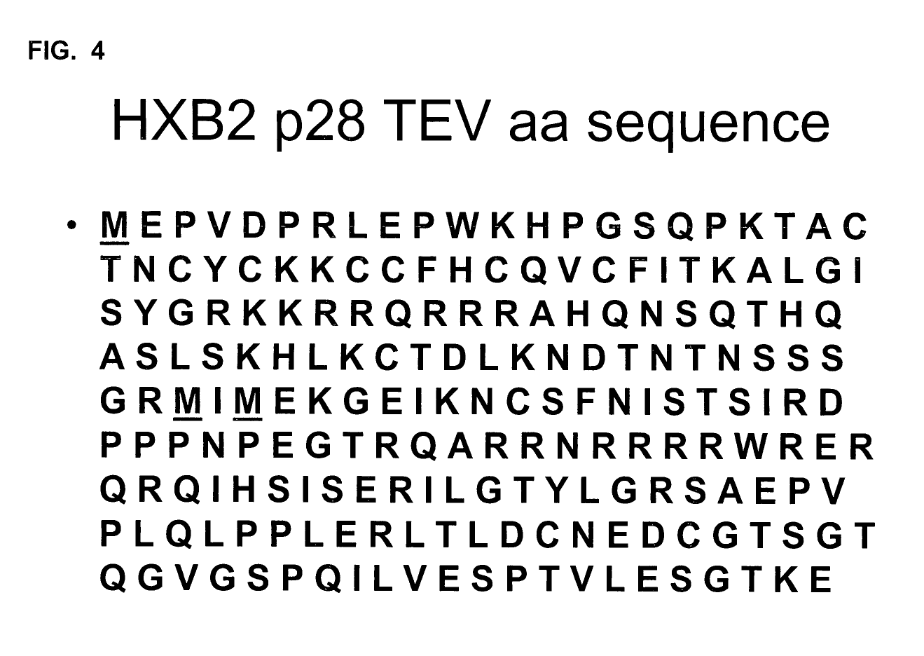

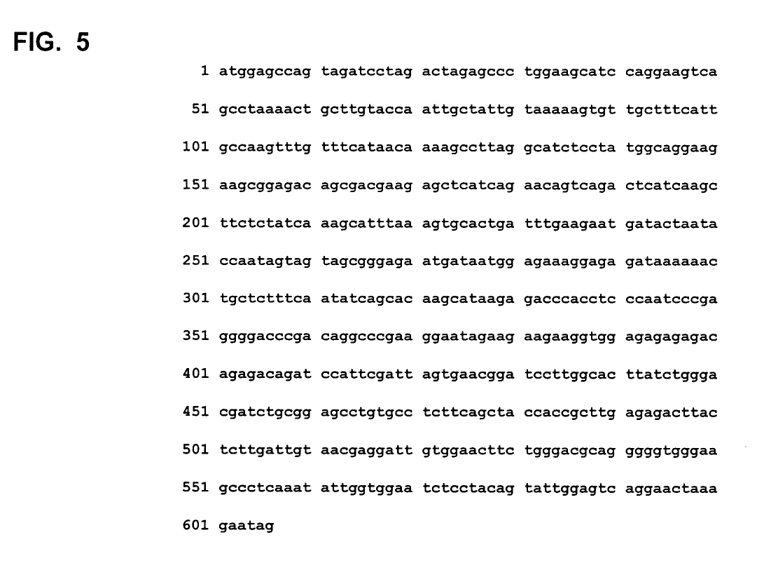

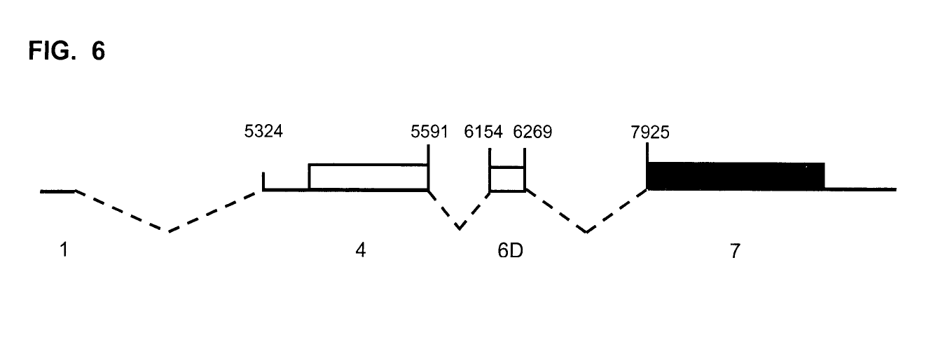

To rule out the possibility that exogenous HIV-1-Luc construct was competing for the transactivation of Tat produced by the HXB2 provirus, the experiments were repeated in the absence of the HIV-1-Luc construct. In the absence of HIV-1-Luc construct, we observed a dose-dependent decrease in p24 production as the amount of p28^{TEV} increased (FIGS. 3A and 3B).

When HXB2 p28^{TEV} is coexpressed in trans with the provirus HXB2, viral production as measured by p24 production is decreased. The overexpression of p28^{TEV} in combination with HXB2 HIV isolate may be acting to inhibit viral production by competing with Rev for RNA binding.

### EXAMPLE 3

### Loss of expression of p28^{TEV}

We have investigated Tev's function in detail by generating three HIV-1 molecular clones in which the acceptor splice site within the Env exon of p28^{TEV} was mutated without altering the amino acid sequence of the Env V1 regio of the protein. This mutation results in the loss of expression p28^{TEV} from the clones.

Preparation of Plasmids

Each of pHXB2, pNL and 89.6 were altered at the acceptor splice site within the ENV exon without altering the ENV amino acid sequence. (See FIGS. 9, 26-33.) The acceptor splice site has a sequence of AGTmTA (SEQ ID NO:13) which is changed to the sequence TCACTG (SEQ ID NO:12) by amplifying the sequences using a PCR primer that changes the splice acceptor site. The sequence of the primer is
5′-CTC TGT GTF TCA CTG AAG TGC ACT-3′ (SEQ ID NO:11).
The change to the acceptor splice site prevents expression of p28^{TEV}. The altered clones are designated pHXB2Δtev, pNL4-3Δtev, and SHIV 89.6Δtev. SHIV89.6 can be obtained from the NIH AIDS Research and Reagent Program. The clone pME was used as a control and was used previously [Nicot C, Dundr M, Johnson J M, Fullen J R, Alonzo N, Fukumoto R, Princler G L, Derse D, Misteli T, Franchini G.HTLV-1-encoded p30¹¹ is a post-transcriptional negative regulator of viral replication. Nat Med. 2004 Feb;10(2):197-201.].

Infectivity Assay

Each of the clones were used to infect TZM or transfect 293T cell cultures. TZM cells are available from the NIH AIDS Research and Reagent program. TZM cells carry luciferase and β galactosidase genes under control HIV-1 promoter. Viral production was measured by detecting p24 production in the supernatant at 24 and 48 hours. The cell cultures were lysed at 24 and 48 hours and run on SDS PAGE gels. The gels were stained with Coomassie blue.

Results

The results are shown in FIGS. 10 and 11. The results in FIG. 10A and B show that the pHXB2Δtev clone results in a decrease of viral infectivity at 24 and 48 hours of cell culture. When cell lysates were run on SDS PAGE (FIGS. 10C and 10D), the HXB2 clone lacking p28^{TEV} expression had increased expression of Rev and decreased expression of p55 and p42.

293T cell cultures were each transfected with pME, pHXB2, pHXB2Δtev, pNL4-3, pNL4-3Δtec, and SHIV89.6. Viral production in the cell cultures were measured at 24 and/or 48 hours as shown in FIG. 10A, 10B and 11A. Transfection of 293T cells with pHXB2Δtev resulted in decreased viral productivity as measured by p24 production. However, pNL4-3Δtev and SHIV89.6tev did not show any decrease in viral production.

The results of the luciferase assay and reserve transcriptase assay show that the supernatant of pHBX2Δtev in TZM cell cultures was less infectious. The expression of luciferase and reverse transcriptase was significantly decreased in the HBX2 clone lacking tev expression. In contrast, as shown in FIG. 11D and 11E, cell cultures infected with pNL4-3Δtev and SHIV89.6Δtev did have comparable activation of Tat-driven luciferase and had equivalent amount of reverse transcriptase activity.

When cell lysates of the cell cultures were analyzed using SDS PAGE, the expression of gp160 was comparable in all cell cultures, except in cells infected with pHXB2Δtev (see FIGS. 10C, 10D and 11B). In the cells infected with pHXB2Δtev, expression of gp160, p55, p42, and p24 were decreased, while rev was greatly increased.

The difference between pHXB2Δtev, pNL4-3Adev and SHIV89.6Δtev is that cell cultures infected with pHXB2Δtev had an increase in Rev expression. The increase in Rev expression may result in inhibition of translation and decrease in production of viral proteins such as reverse transcriptase, gp160 and p24.

The effect of increasing amounts of Rev on translation was determined by adding an increasing amount of cDNA encoding Rev to 293 cells transfected with pNL4-3Δtev. The results are shown in FIG. 11C. When increasing amounts of Rev were added to the cells, p24 productivity decreased.

### EXAMPLE 4

### DNA Plasmids Expressing p28^{TEV}

We generated three DNA plasmids expressing Tev from the CCR5 users SF162, BaL, and the CXCR4 user simian-human immunodeficiency virus (SHIV)-89.6P and demonstrated their expression in E. coli cells (FIG. 12A) and compared their protein products to the pHXB2 Tev obtained by Benko et al. The DNA plasmid constructs were produced as described below. The 89.6P protein was expressed in E. coli and purified (FIG. 12B) using conventional techniques.

Plasmid preparation

Synthetic genes encoding p28^{tev} from each of BaL, SF162, HXB2, and 89.6 were prepared using synthetic oligonucloetides and codons were optimized for human and E. coli cells. The sequences of the genes are provided in FIGS. 26-33. The genes were cloned into a vector pPCR-Script-Amp (Stratagene, Calif.) using Kpn1 and Sac1 restriction sites as shown in FIGS. 34A-D. The plasmid DNA was purified and concentration determined by UV spectroscopy. The final construct was verified by resequencing as shown in FIGS. 26-33.

Purification of p28^{tev}

Recombinant HIV-1 89.6 p28^{tev} was purified from E. Coli on mouse anti-tat sepharose. Mouse anti-tat sepharose can be prepared using conventional methods. The fractions containing p28^{tev} were then run on anti-mouse IgG sepharose (available from Invitrogen, Calif.) to remove any antibodies that may have leached out from the first column. The p28^{tev} fractions were passed through a polymixin B column to reduce endotoxin levels to about 436 EU/mg. The tev protein was then filtered and a sample was run on an SDS-PAGE.

Results

As shown in FIGS. 12A and 12B, the DNA plasmids SF162, Bal, and 89.6P each express p28^{TEV} DNA plasmid 89.6P was introduced into E. coli and p28^{TEV} was produced and isolated. (FIG. 12B)

### EXAMPLE 5

### Immunization Protocols

The BaL, SF162, 89.6P plasmid DNAs were used to immunize Rhesus macaques and the purified protein from 89.6P used to boost antibodies and other virus-specific immune responses as indicated in FIG. 12C. Animals were immunized with DNA plasmids encoding p28^{TEV} at 0, 3, and 8 months. Booster injections of p28^{TEV} polypeptide 89.6 in combination with an immunomodulator CpG were administered at 12 and 18 months. At 22 weeks, the animals were challenged with SHIV89.6P.

Plasma antibody titers for Tev, IIIB Tat, HIV_{MN} Rev, and Env V1 peptide pool were determined in the immunized animals at 5, 10, 14, 19, 22, 27 and 30 weeks. Viral load in the animals was measured post challenge at 22, 24, 26, 28, 30, 32, 34, 35, 36, 38, 40, 42, 44 and 46 weeks. CD3/4 cell counts were also taken post challenge at the same time periods. Antibody titers were determined in ELISA or ELISPOT assays. Control animals received CpG rejections at 12 and 18 months.

Viral Load Assay

A number of different assays are available for determining viral load from biological samples, such as serum or tissues. Assays such as the Amplicore assay available from Roche Diagnostics is a PCR-based assay that can detect SHIV RNA. The assay utilized was the NASBA assay as described by Romano et al., Immunological Investigations, 26:15-28 (1997). NASBA assay kits for HIV-1 are available as NucleoSens Easy Q® HIV-1, V1.1 (BioMerieux, France). A reprsentative NASBA assay is described below.

Briefly, one volume of tissue or seum is added to 9 volumes of lysio buffer (5.25 MGuSCN, 50 mM Tris, ph 7.2, 20 mM EDTA, 1.3% Triton X-100). Next 50 ul of acid treated silica is added to the lysate and is pelleted by centrifugation and washed. The pellet is washed twice with ethanol and once with acetone. The acetone is evaporated from the silica and the nucleic acids are eluted from the silica into water.

The amplification reaction includes 5 ul nucleic acid extract, 10 ul of 80 mM Tris, ph 8.3, 24 mM MgCl₂, 140 mM KCL, 10 mM DTT, 2.0 mM each dNTP, 4 mM each NTP, 30% DMSO, 0.4 uM Primer 1 and 0.4 uM primer 2. HIV primers are described in Van Gemen, J. Virol. Meth., 49:157 (1994) and herein. This mix is heated to 65° C. for 5 minutes and then cooled to 41° C. Once cooled, 5 ul of enzyme mix (6.4 units/ul T7 RNA polymerase, 1.3 units/ul AMU-RT, 0.02 units, 1 ul RNase H, 0.42 ug/ul BSA) is added and incubated for 90 minutes at 41° C. The reaction product is then characterized by hybridization analysis.

Probes can be labeled with a detectable label and then used in southern blots, enzyme linked gel assays, or electrochemoluminescense systen to detect the reaction products.

CD3/CD4 Cell Count

White blood cell counts in vaccinated and control animals after challenge with SHIV89.6 were conducted at two week intervals from 22 weeks to 44 weeks. CD3/CD4⁺ positive cells were quantitated using flow cytometry or ELISPOT assays.

Identification of effector CD4⁺ and CD8⁺ T cells.

Fresh peripheral blood mononuclear cells (PBMCs) were isolated using LSM (Lymphocyte Separation Medium) (Cappel, Aurora, Ohio) density centrifugation. In some instances PBMC were frozen (90% fetal calf serum (FSC) 10% DMSO) until use.

10⁶ PBMC were incubated with 1 μg/ml each of antibodies CD28 and anti-CD49d and 1 μg/ml of Gag pool peptide in a 1-ml volume. Conjugated antibodies to the granular membrane proteins CD107a were kindly supplied by M. R. Betts (Laboratory of Immunology, Vaccine Research Center, National Institute of Allergy and Infectious Diseases, National Institutes of Health, Bethesda, Md.) and were added to the cells prior to stimulation. A negative control (anti-CD28, anti-CD49d), and positive control (SEB, 1 μg/ml) were included to control for spontaneous, and activated production production of cytokines and/or expression of CD107a. The cultures were incubated for 1 h at 37° C. in 5% CO₂, followed by an additional 4 hours in the presence of the secretion inhibitor monensin (BD Pharmingen) and Brefeldin A (Sigma). After stimulation, PBMC were washed, and surface stained (anti-CD4, BD Pharmingen); anti-CD8β, Immunotech), washed again, and after then permeabilized. After permeabilization, the cells were washed and stained with antibodies specific for intracellular markers (IFN-γ and TNF-α monoclonal antibodies, BD Pharmingen). The cells were washed a final time and resuspended in 1% paraformaldehyde (Electron Microscopy Systems, Fort Washington, Pa.) in PBS. Four-parameter flow cytometry analysis was performed using a FACSCalibur flow cytometer (Becton Dickinson Immunocytometry Systems). List mode data files were analyzed using FlowJo software (Tree Star, San Carlos, Calif.). In all cases at least 100.000 live events were collected for analysis.

ELISPOT Assays

Immunospot plates (Cellular Technology, Cleveland, Ohio) were coated overnight with capture antibodies cross-reactive with macaque IFN-γ. The plates were then blocked with BSA (10 g/l in PBS) for 1 hour and washed three times with PBS. Cells were plated in complete RPM1. Cells that were CD3^{+/CD}4⁺were separated from other T cells and were quantitated by flow cytometry with anti-CD3 and anti-CD4 antibodies. The p28^{TEV} overlapping peptide pools were added at a concentration of 1 μg/ml. After 24 h, the plates were washed and biotinylated. Antibodies were added for 12 hours at 4° C. Subsequently, strepavidin-HRP was added for 2 hours at room temperature. The spots were developed using AEC (Pierce Pharmaceuticals).

Macaque IFN-γ-specific ELISPOT kits manufactured by U-Cytech (Utrecht, Netherlands) were used in order to detect the antigen-specific number of WFN-y-producing cells. Ninety-six-well flat-bottom plates were coated with anti-IFN-γ monoclonal antibody MD-1 overnight at 4° C. and blocked with 2% body surface area in PBS for 1-3 hours at 37° C. 1×10⁵ cells/well were loaded in triplicate in RPMI-1640 containing 5% human serum and 1 μg/ml of a specific peptide or 5 μg/ml concanavalin A as a positive control. The plates were incubated overnight at 37° C., 5% CO₂, and developed according to the manufacturer's guidelines (U-Cytech). In cases where intracellular IFN-γ production was measured in the presence of feeder cells, 10⁵ CD8-depleted splenocytes were added to the cells to be examined in each well. CD8+ cells were removed from Ficoll-purified splenocytes using αCD8 antibody coated beads (Dynal, Oslo, Norway) and contained less than 0.3% of residual CD3+ CD8+ cells. In this assay, results were considered positive at more than 25 spot-forming cells/10⁶ cells, based on background levels obtained with cells from Mamu-A*01-negative animals using Mamu-A*01-restricted CTLs) epitopes or stimulation with irrelevant peptides.

ELISA Assay

ELISA assays were conducted on serum samples from immunized and control animals both before and after challenge with SHIV98.6. Antibodies to Tat, Rev and VI were obtained from Advanced BioScience Laboratories, Inc., 5510 Nicholson Lane, Kensington, Md. 20895.

Results

The data presented in FIG. 13A demonstrated plasma virus levels following challenge exposure in vaccinated animals (top panel) and controls (FIG. 13B). Controls consisted of two animals challenged simultaneously with the vaccinated animals and four additional animals that were historical controls challenged in exactly the same way using the identical virus stock.

Three out of four animals of the vaccinated group were able to control viral replication. These animals can be ranked in their ability to control viral replication as 316 first, 308 second, and 490 third (top panel, FIG. 13A). As expected, these three animals were protected from CD4+ T cell loss (FIG. 14A).

The levels of SIV RNA as measured by copies/μg RNA in various tissues and control animals were assessed at the time of sacrifice (please identify the time). The level of SIV RNA in plasma was measured per 100 ul of plasma for immunized animals and 500 ul plasma for control animals. The results are shown in FIG. 24. The results show that the immunized animals 308 M and 316 M that controlled viral replication had about 3 logs decrease of virus levels in lymph nodes, jejunum, spleen, and plasma as compared to control animals (320 M; 915 L). In contrast, the immunized animal 218 that did not control viral replication as well showed some decrease of 1-2 logs in viral levels in most tissues as compared to control animals.

This platform (DNA/protein boost) was chosen to favor the elicitation of antibodies over T cell response. Indeed, negligible ELISPOT responses were observed at the time of challenge (week 22). However, these responses were boosted by the challenge virus in the vaccinated animals (FIG. 15), especially in response to the 89.6 pool of p28^{TEV} polypeptides. In control animals, no ELISPOT responses were observed, compatible with the fast and irreversible CD4 depletion that occurs following infection with 89.6P likely impairing the CD8+ T cell function. Animal 218 mounted a broad response to the Tev peptides derived from HIV_{BAL}, HIV_{SF2}, and HIV_{89.6P}, likely because it remained viremic during the observation time (FIG. 15). Animal 308 responded well to peptides from HIV_{89.6P} and less so to HIV_{BAL} and HIV_{SF2} (FIG. 15). Animals 316 responded poorly to all peptides in the pool, possible because viral replication was blunted early. Correlative analysis of T cell response and the level of virus following challenge exposure demonstrated no correlation with T cell response and control of virus replication.

As our intention was to induce antibodies to V1, we measured the relative level of antibodies induced by our regimens in the sera of macaques before and after challenge exposure. ELISA titers were measured against the Tat, Rev, and the pool of V1 peptides (SF162, BAL, 89.6P). All vaccinated animals mounted titers of antibodies to Tat in the range of 1.2×10⁴ to 3.5×10⁴, and, at the time of challenge, they had equivalent titers of antibodies (see FIG. 16). Surprisingly, no anamnestic response to Tat was observed. Low levels of antibody to Rev were observed (FIG. 17), particularly in macaques 490 and 218. As in the case of Tat, Rev antibodies were not boosted by viral infection. In contrast, antibody titers to V1 ranging between 6×10³ and 3.5×10⁴ were elicited by vaccination and boosted by viral challenge in three of the macaques that were protected against high virus load in the long term, CD4+ T cell loss, and death. Macaque 218 that behaved like a control animal had no detectable antibodies following challenge exposure (FIG. 18). Similarly, control macaques had background levels of antibodies to V1 (FIG. 18).

Example 6

To further confirm these data and to assess which region of V1 was reactive to the sera of the immunized macaques, we performed peptide scan analysis on all animal sera. 15-mer peptides, overlapping by 11 aa, derived from the cDNA amino acid sequence of the p28^{tev} protein of 89.6 (see FIG. 33) were synthesized. The purity of the peptides was >70%, as determined by HPLC and mass spectrophotometry, other contaminants being incomplete peptides. The peptides diluted in 5 mM HEPES (pH 7) were added to 96-well plates (Dynatech) in volumes of 10 μl per well at a final concentration of 0·3 μmol per well.

These assays demonstrated that, while all animals recognized similar peptides within Tat and Rev, they differed in their ability to respond to the V1 region (FIGS. 19-23). Indeed, the three animals that fared better and were able to control viremia recognized peptides spanning 21-24, which correspond to the V1 region (FIGS. 19-23). As expected, these peptides within the V1 region were not recognized by the sera of control animals 915 and 320 (FIG. 22). It is worth noting that the animal that had the highest reactivity to peptides 21, 22, and 23 (1:5×10³) was animal 316 that fared better and was able to control virus replication by week 6 from SHIV_{89.6P} exposure (FIG. 21).

As we had immunized with a mixture of cDNA and boosted only with the SHIV_{89.6P} protein, we wished to investigate whether the reactivity of the animal sera was type-specific. The sera from animals 316, 308, and 490 recognized peptides 21-24 of the 89.6P V1 region (FIG. 23A). In contrast, peptides 21-24 from the HIV_{BAL} and HIV_{SF162} were not recognized by any of the animal sera (FIGS. 23B and C).

Example 7

### Antibody Dependent Cellular Cytotoxicity

Serum from the immunized animals and control animals post challenge were analyzed for the ability to participate in antibody dependent cellular cytotoxicity of HIV infected cells.

The assay was conducted with PHA stimulated primary human CD4 blasts and primary human monocytes effectors at a ratio of E:T of 1:1. Plasma samples were added at a final dilution of 1:100. Peripheral blood monocyte cells were obtained from human whole blood in heparin sulfate. The cells were incubated with 3 to 5 ug of PHA/mol for 48 hours. CD4+ cells were purified by positive selection with magenetic beads (Stem Cell Technologies, Vancouver, B.C., Canada). Primary human monocyte effects were obtained from healthy HIV seronegative donors.

The CD4+ lymphocytes were infected with SHIV89.6 (virus isolate) at a multiplicity of infection of 0.5. The infected cells were then incubated with plasma from immunized and control animals. Effector cells at an E :T ratio of 1:1 were added. The cytotoxicity of the cells was measured using inhibition of p24 production.

The results are shown in FIG. 25. The results in FIG. 25 show that plasma from at least two of the immunized animals 308 and 490 showed an increase antibody mediated cellular cytotoxicity post challenge with SHIV 89.6P. These two animals were effective at controlling viral replication. The immunized animal 316 showed a slight increase in ADCC post challenge. One of the control animals also demonstrated an ADCC response post challenge.

These results indicate that antibodies specific to the V1 region of p28^{TEV} may in part control viral replication by an antibody dependent cellular cytotoxicity mechanism.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

All publications and patent applications in this specification are indicative of the level of ordinary skill in the art to which this disclosure pertains. All publications and patent applications are herein incorporated by reference to the same extent as if each individual publication or patent application was specifically and individually indicated by reference.

The disclosure has been described with reference to various specific and preferred embodiments and techniques. However, it should be understood that many variations and modifications may be made while remaining within the spirit and scope of the disclosure.

What is claimed is:

1. A immunogenic composition comprising an effective amount of at least one isolated nucleic acid encoding at least one p28^{TEV} polypeptide having at least about 70 percent amino acid sequence identity with an amino acid sequence selected from the group consisting of SEQ ID NO:143, SEQ ID NO:146, SEQ ID NO:1, and fragments thereof.

2. The immunogenic composition of claim 1, wherein the nucleic acid further encodes a second HIV antigen.

3. The immunogenic composition of claim 2, wherein the second HIV antigen is selected from the group consisting of Vif, Tat, Gag, Env, Rev, gp120, gp41, p24, p7, p17, or an immunogenic fragment thereof.

4. The immunogenic composition of claim 1, wherein the nucleic acid further encodes an immunomodulator for increasing the immunogenic response.

5. The immunogenic composition of claim 1, wherein the composition comprises at least one adjuvant.

6. The immunogenic composition of claim 1, wherein the nucleic acid comprises a recombinant viral vector.

7. The immunogenic composition of claim 1, wherein the nucleic acid encodes at least two different p28^{TEV} polypeptides from different HIV clades.

8. The immunogenic composition of claim 1, wherein the nucleic acid comprises a recombinant bacterial vector.

9. The immunogenic composition of claim 1, wherein the composition comprises at least two nucleic acids, each nucleic acid encoding a p28^{TEV} polypeptide from a different HIV clade.

10. The immunogenic composition of claim 1, wherein the nucleic acid is at least one naked DNA.

11. The immunogenic composition of claim 1, wherein the polypeptide comprises a sequence having at least about 80 percent amino acid sequence identity with an amino acid sequence of SEQ ID NO:151.

12. The immunogenic composition of claim 1, wherein the polypeptide comprises an amino acid sequence having at least about 90 percent amino acid sequence identity with an amino acid sequence of SEQ ID NO:151.

13. An immunogenic composition comprising an immunogenic effective amount of at least one isolated and purified polypeptide comprising an amino acid sequence having at least about 70 percent amino acid sequence identity with the p28^{TEV} polypeptide with the amino acid sequence selected from the group consisting of SEQ ID NO:143, SEQ ID NO:146, SEQ ID NO:151, SEQ ID NO:1, and fragments thereof.

14. The immunogenic composition of claim 13, wherein the polypeptide comprises an amino acid sequence having at least about 80 percent amino acid sequence identity with the polypeptide with the amino acid sequence of SEQ ID NO:151.

15. The immunogenic composition of claim 13, wherein the polypeptide comprises an amino acid sequence having at least about 90 percent amino acid sequence identity with the polypeptide with the amino acid sequence of SEQ ID NO:151.

16. The immunogenic composition of claim 13, wherein the polypeptide comprises an amino acid sequence of SEQ ID NO:151.

17. The immunogenic composition of claim 13, comprising at least two isolated and purified p28^{TEV} polypeptides from at least two HIV-1 clades.

18. The immunogenic composition of claim 13, further comprising an immunomodulator or adjuvant.

19. The immunogenic composition of claim 1, wherein the nucleic acid encodes a polypeptide that has at least 90% sequence identity to the polypeptide comprising the sequence selected from the group consisting of SEQ ID NO:136, SEQ ID NO:137, SEQ ID NO:138, SEQ ID NO:139, SEQ ID NO:140, SEQ ID NO:152, SEQ ID NO:153, SEQ ID NO:154, SEQ ID NO:155, SEQ ID NO:156, and mixtures thereof.

20. The immunogenic composition of claim 13, wherein the polypeptide has at least 90% sequence identity to the polypeptide comprising the sequence selected from the group consisting of SEQ ID NO:136, SEQ ID NO:137, SEQ ID NO:138, SEQ ID NO:139, SEQ ID NO:140, SEQ ID NO:152, SEQ ID NO:153, SEQ ID NO:154, SEQ ID NO:155, SEQ ID NO:156, and mixtures thereof.

21. A method for reducing HIV viral levels in a host comprising:
administering a composition of claim 1 to a mammal.

22. The method of claim 21, further comprising subsequently administering the composition of claim 13 to the mammal.

23. An antibody that specifically binds: a) a naturally occurring p28^{TEV} polypeptide comprising an amino acid sequence selected from the group consisting of SEQ ID NO:143, SEQ ID NO:146, SEQ ID NO:151, SEQ ID NO:1, and fragment thereof, or b) a polypeptide having an amino acid sequence about 90% identical to a polypeptide comprising an amino acid sequence of SEQ ID NO:1, SEQ ID NO:143, SEQ ID NO;145, SEQ ID NO:151, and fragment thereof.

24. The antibody of claim 23, wherein the antibody is generated or selected using a polypeptide having at least 90% sequence identity to the polypeptide comprising the sequence selected from the group consisting of SEQ ID NO:136, SEQ ID NO:137, SEQ ID NO:138, SEQ ID NO:139, SEQ ID NO:140, SEQ ID NO:152, SEQ ID NO:153, SEQ ID NO:154, SEQ ID NO:155, SEQ ID NO:156, and mixtures thereof.

25. An antagonist of p28^{TEV} polypeptide, comprising an isolated polynucleotide that inhibits expression of p28^{TEV}.

26. The antagonist of claim 25, wherein the polynucleotide is an antisense RNA, small interfering RNA (siRNA) molecule, or a microRNA molecule.

27. The antagonist of claim 25, wherein the polynucleotide hybridizes under moderately stringent conditions to a nucleic acid molecule encoding p28^{TEV}.

28. The antagonist of claim 25, wherein the polynucleotide comprises a polynucleotide sequence of SEQ ID NO:2.

29. The antagonist of claim 25, wherein the polynucleotide hybridizes under moderately stringent conditions to a nucleic acid molecule encoding a p28^{TEV} splice acceptor site or p28^{TEV} splice donor site.

30. The antagonist of claim 29, wherein the nucleic acid sequence comprises a polynucleotide sequence encoding an Env exon.

31. The antagonist of claim 30, wherein the exon is exon 6D.

32. The antagonist of claim 29, wherein the p28^{TEV} splice acceptor site comprises a polynucleotide sequence of SEQ ID NO:3.

33. The antagonist of claim 29, wherein the p28^{TEV} splice donor site comprises a polynucleotide sequence of SEQ ID NO:4.

34. A vector comprising the polynucleotide of claim 25.

35. A host cell comprising the vector of claim 34.

36. A method for detecting HIV in a biological sample, comprising:
a) contacting a biological sample with at least one antibody that specifically binds a p28^{TEV} polypeptide; and
b) detecting the presence of p28^{TEV} in the biological sample.

37. A method for identifying an antagonist of p28^{TEV} polypeptide, comprising:
a) contacting a p28^{TEV} polypeptide or cell comprising a polynucleotide encoding a p28^{TEV} polypeptide cell with a candidate agent; and
b) determining whether the candidate agent reduces HIV viral levels or transcriptional activity of the p28^{TEV} polypeptide, wherein the candidate agent that inhibits the HIV viral levels or transcriptional activity of p28^{TEV} polypeptide is identified as an antagonist of the p28^{TEV} polypeptide.

38. A method for inhibiting HIV viral levels, comprising administering to a patient in need thereof an effective amount of an antagonist of expression or activity of a p28^{TEV} polypeptide.

39. The method of claim 38, wherein the antagonist is an antibody.

40. The method of claim 39, wherein the antibody is humanized.

41. The method of claim 40, wherein the antibody binds to the V1 region of p28^{TEV}.

42. The method of claim 41, wherein the antibody is attached to a siRNA molecule or cytotoxic molecule.

43. The method of claim 41, wherein the antibody is a Fv, Fab, Fab′, or F(ab′)₂ fragment.

44. The method of claim 38, wherein the antagonist is a polynucleotide that inhibits expression of p28^{TEV}.

45. The method of claim 44, wherein the polynucleotide is an antisense RNA or a small interfering RNA (srRNA) molecule.

46. The method of claim 45, wherein the polynucleotide hybridizes under stringent conditions to a nucleic acid molecule encoding p28^{TEV}.

47. The method of claim 46, wherein the nucleic acid molecule comprises a polynucleotide sequence of SEQ ID NO:2.

48. The method of claim 46, wherein the polynucleotide hybridizes under stringent conditions to a nucleic acid molecule encoding a p28^{TEV} splice acceptor site or p28^{TEV} splice donor site.

49. The method of claim 38, wherein the patient is infected with HIV.

50. The method of claim 21, further comprising administering an antiviral agent.

51. The method of claim 50, wherein the antiviral agent is selected from the group consisting of nucleoside reverse transcriptase inhibitors, nonnucleoside reverse transcriptase inhibitors, protease inhibitors, fusion inhibitors, or attachment inhibitors.

52. The antibody of claim 23, wherein the antibody is monoclonal.

53. The antibody of claim 52, wherein the antibody is a humanized antibody.

54. The antibody of claim 23, wherein the antibody is Fv Fab, Fab′, or F(ab′)₂.

55. The antibody of claim 23, wherein the antibody is attached to a detectable label, cytotoxic agent, or siRNA molecule.
